(19) United States
(12) Patent Application Publication
(10) Pub. No.: US 2021/0253666 A1
(43) Pub. Date: Aug. 19, 2021

(54) COMPOSITIONS AND METHODS FOR TCR REPROGRAMMING USING FUSION PROTEINS

(71) Applicant: TCR2 THERAPEUTICS INC., Cambridge, MA (US)

(72) Inventors: Patrick Alexander Baeuerle, Gauting (DE); Robert Hofmeister, Scituate, MA (US); Jian Ding, Newton, MA (US); Vania Ashminova, North Billerica, MA (US); Michael Lofgren, Watertown, MA (US)

(21) Appl. No.: 17/271,265

(22) PCT Filed: Aug. 30, 2019
(86) PCT No.: PCT/US2019/049202

§ 371 Date: Feb. 25, 2021

Related U.S. Application Data
(60) Provisional application No. 62/725,098, filed on Aug. 30, 2018.

(51) Int. Cl. C07K 14/725; C07K 16/30; C12N 15/86; A61K 35/17

(52) U.S. Cl. CPC C07K 14/7051; C07K 16/3092; A61K 38/00; A61K 35/17; C12N 15/86

(57) ABSTRACT

Provided herein are T cell receptor (TCR) fusion proteins (TFPs) having specificity for more than one tumor cell associated antigen, T cells engineered to express one or more TFP, and methods of use thereof for the treatment of diseases, including cancer.

### CROSS-REFERENCE

This application claims the benefit of U.S. Provisional Patent Application No. 62/725,098, filed Aug. 30, 2018, which is entirely incorporated herein by reference.

### BACKGROUND

Most patients with hematological malignancies or with late-stage solid tumors are incurable with standard therapy. In addition, traditional treatment options often have serious side effects. Numerous attempts have been made to engage a patient's immune system for rejecting cancerous cells, an approach collectively referred to as cancer immunotherapy. However, several obstacles make it rather difficult to achieve clinical effectiveness. Although hundreds of so-called tumor antigens have been identified, these are often derived from self and thus can direct the cancer immunotherapy against healthy tissue, or are poorly immunogenic. Furthermore, cancer cells use multiple mechanisms to render themselves invisible or hostile to the initiation and propagation of an immune attack by cancer immunotherapies.

Recent developments using chimeric antigen receptor (CAR) modified autologous T cell therapy, which relies on redirecting genetically engineered T cells to a suitable cell-surface molecule on cancer cells, show promising results in harnessing the power of the immune system to treat cancers. For example, the clinical results from an ongoing trial with B-cell maturation antigen (BCMA)-specific CAR T cells have shown partial remission in some multiple myeloma patients (one such trial may be found via clinicaltrials.gov identifier NCT02215967). An alternative approach is the use of T cell receptor (TCR) alpha and beta chains selected for a tumor-associated peptide antigen for genetically engineering autologous T cells. These TCR chains will form complete TCR complexes and provide the T cells with a TCR for a second defined specificity. Encouraging results were obtained with engineered autologous T cells expressing NY-ESO-1-specific TCR alpha and beta chains in patients with synovial carcinoma.

Besides the ability of genetically modified T cells expressing a CAR or a second TCR to recognize and destroy respective target cells in vitro/ex vivo, successful patient therapy with engineered T cells requires the T cells to be capable of strong activation, expansion, persistence over time, and, in case of relapsing disease, to enable a ‘memory’ response. High and manageable clinical efficacy of CAR T cells is currently limited to mesothelin-positive B cell malignancies and to NY-ESO-1-peptide-expressing synovial sarcoma patients expressing HLA-A2. There is a clear need to improve genetically engineered T cells to more broadly act against various human malignancies. Described herein are novel fusion proteins of TCR subunits, including CD3 epsilon, CD3 gamma and CD3 delta, and of TCR alpha and TCR beta chains with binding domains specific for cell surface antigens that have the potential to overcome limitations of existing approaches. Described herein are novel fusion proteins that more efficiently kill target cells than CARs, but release comparable or lower levels of pro-inflammatory cytokines. These fusion proteins and methods of their use represent an advantage for TFPs relative to CARs because elevated levels of these cytokines have been associated with dose-limiting toxicities for adoptive CAR-T therapies.

### SUMMARY

Provided herein are binding proteins having specificity for more than one target, and antibodies and T cell receptor (TCR) fusion proteins (TFPs) comprising such dual-specificity binding proteins. In addition are provided T cells engineered to express one or more TFPs, and methods of use thereof for the treatment of diseases. The TFPs may have dual specificity on a single molecule, or in a single engineered TCR; alternatively, the dual specificity may come from mixing two engineered T cell populations comprising the TFPs, or transducing a single population of T cells with two different viruses.

Thus, in one aspect is provided a composition comprising an isolated recombinant nucleic acid molecule encoding a first T cell receptor complex (TCR) fusion protein (TFP) comprising: a TCR subunit comprising at least a portion of a TCR extracellular domain, a transmembrane domain, and an intracellular domain comprising a stimulatory domain from an intracellular signaling domain derived only from a TCR subunit selected from the group consisting of a TCR alpha chain, a TCR beta chain, a TCR gamma chain, a TCR delta chain, a CD3 gamma chain, a CD3 delta chain and a CD3 epsilon chain; and a murine, human, or humanized antibody domain comprising an anti-MUC16 binding domain, wherein the TCR subunit and the anti-MUC16 binding domain are operatively linked, wherein the first TFP functionally interacts with a TCR or incorporates into a TCR when expressed in the T cell; and a second recombinant nucleic acid sequence encoding a second TFP comprising a TCR subunit comprising at least a portion of a TCR subunit extracellular domain, a transmembrane domain, and (iii) a TCR intracellular domain comprising a stimulatory domain from an intracellular signaling domain derived only from a TCR subunit selected from the group consisting of a TCR alpha chain, a TCR beta chain, a TCR gamma chain, a TCR delta chain, a CD3 gamma chain, a CD3 delta chain and a CD3 epsilon chain; and (b) a murine, human or humanized antibody domain comprising an anti-mesothelin (MSLN) binding domain, wherein the TCR subunit and the anti-MSLN binding domain are operatively linked, wherein the second TFP functionally interacts with a TCR or incorporates into a TCR when expressed in a T cell.

In another aspect is provided a composition comprising a first recombinant nucleic acid sequence encoding a first T cell receptor (TCR) fusion protein (TFP) comprising a TCR subunit comprising at least a portion of a TCR extracellular domain, a transmembrane domain, and a TCR intracellular domain comprising a stimulatory domain from an intracellular signaling domain derived only from a TCR subunit selected from the group consisting of a TCR alpha chain, a TCR beta chain, a TCR gamma chain, a TCR delta chain, a CD3 gamma chain, a CD3 delta chain and a CD3 epsilon chain; and a first human or humanized antibody domain comprising an anti-MUC16 binding domain and a second human or humanized antibody domain comprising an anti-MSLN binding domain, wherein the TCR subunit, the first antibody domain, and the second antibody domain are operatively linked, and wherein the first TFP functionally interacts with a TCR or incorporates into a TCR when expressed in a T cell.

In another aspect is provided a composition comprising an isolated recombinant nucleic acid molecule encoding a first T cell receptor (TCR) fusion protein (TFP) comprising a TCR subunit, a first human or humanized antibody domain comprising a first antigen binding domain that is an anti-MUC16 binding domain; and a second T cell receptor (TCR) fusion protein (TFP) comprising a TCR subunit, a second human or humanized antibody domain comprising a second antigen binding domain that is an anti-MSLN binding domain, wherein the TCR subunit of the first TFP and the first antibody domain are operatively linked and the TCR subunit of the second TFP and the second antibody domain are operatively linked.

In another aspect is provided a composition comprising an isolated recombinant nucleic acid molecule encoding a first T cell receptor (TCR) fusion protein (TFP) comprising a TCR complex subunit, a first human or humanized antibody domain comprising a first antigen binding domain that is an anti-MUC16 binding domain and a second human or humanized antibody domain comprising a second antigen binding domain that is an anti-MSLN binding domain; and wherein the TCR subunit of the first TFP, the first antibody domain and the second antibody domain are operatively linked.

In one embodiment, the extracellular, transmembrane, and intracellular signaling domains of the encoded TCR subunit of the first TFP are derived only from a TCR subunit selected from the group consisting of a TCR alpha chain, a TCR beta chain, a TCR gamma chain, a TCR delta chain, a CD3 gamma chain, a CD3 delta chain and a CD3 epsilon chain. In another embodiment the extracellular, transmembrane, and intracellular signaling domains of the TCR subunit of the second TFP are derived only from a TCR subunit selected from the group consisting of a TCR alpha chain, a TCR beta chain, a TCR gamma chain, a TCR delta chain and a TCR epsilon chain. In another embodiment, the extracellular, transmembrane, and intracellular signaling domains of the TCR subunit of the first TFP are derived only from a TCR alpha chain. In another embodiment, the extracellular, transmembrane, and intracellular signaling domains of the TCR subunit of the first TFP are derived only from a TCR beta chain. In another embodiment, the extracellular, transmembrane, and intracellular signaling domains of the TCR subunit of the first TFP are derived only from a TCR gamma chain. In another embodiment, the extracellular, transmembrane, and intracellular signaling domains of the TCR subunit of the first TFP are derived only from a TCR delta chain. In another embodiment, the extracellular, transmembrane, and intracellular signaling domains of the TCR subunit of the first TFP are derived only from a CD3 gamma chain. In another embodiment, the extracellular, transmembrane, and intracellular signaling domains of the TCR subunit of the first TFP are derived only from a CD3 delta chain. In another embodiment, the extracellular, transmembrane, and intracellular signaling domains of the TCR subunit of the first TFP are derived only from a CD3 epsilon chain.

In another embodiment, the extracellular, transmembrane, and intracellular signaling domains of the TCR subunit of the second TFP are derived only from a TCR alpha chain. In another embodiment, the extracellular, transmembrane, and intracellular signaling domains of the TCR subunit of the second TFP are derived only from a TCR beta chain. In another embodiment, the extracellular, transmembrane, and intracellular signaling domains of the TCR subunit of the second TFP are derived only from a TCR gamma chain. In another embodiment, the extracellular, transmembrane, and intracellular signaling domains of the TCR subunit of the second TFP are derived only from a TCR delta chain. In another embodiment, the extracellular, transmembrane, and intracellular signaling domains of the TCR subunit of the second TFP are derived only from a CD3 gamma chain. In another embodiment, the extracellular, transmembrane, and intracellular signaling domains of the TCR subunit of the second TFP are derived only from a CD3 delta chain. In another embodiment, the extracellular, transmembrane, and intracellular signaling domains of the TCR subunit of the second TFP are derived only from a CD3 epsilon chain.

In one embodiment, the first TFP, the second TFP, or both incorporate into a TCR or functionally interact with a TCR when expressed in a T cell. In another embodiment, the first TFP, the second TFP, or both incorporate into a TCR or functionally interact with a TCR when expressed in a T cell. In another embodiment, the encoded first antigen binding domain is connected to the TCR extracellular domain of the first TFP by a first linker sequence, the encoded second antigen binding domain is connected to the TCR extracellular domain of the second TFP by a second linker sequence, or both the first antigen binding domain is connected to the TCR extracellular domain of the first TFP by the first linker sequence and the encoded second antigen binding domain is connected to the TCR extracellular domain of the second TFP by the second linker sequence. In another embodiment, the first linker sequence and the second linker sequence comprise (G₄S)ₙ, wherein n=1 to 4. In another embodiment, the TCR subunit of the first TFP, the TCR subunit of the second TFP, or both comprise a TCR extracellular domain. In another embodiment, the TCR subunit of the first TFP, the TCR subunit of the second TFP, or both comprise a TCR transmembrane domain. In another embodiment, the TCR subunit of the first TFP, the TCR subunit of the second TFP, or both comprise a TCR intracellular domain. In another embodiment, the TCR subunit of the first TFP, the TCR subunit of the second TFP, or both comprise (i) a TCR extracellular domain, (ii) a TCR transmembrane domain, and (iii) a TCR intracellular domain, wherein at least two of (i), (ii), and (iii) are from the same TCR subunit. In another embodiment, the TCR subunit of the first TFP, the TCR subunit of the second TFP, or both comprise a TCR intracellular domain comprising a stimulatory domain selected from an intracellular signaling domain of CD3 epsilon, CD3 gamma or CD3 delta, or an amino acid sequence having at least one modification thereto. In another embodiment, the TCR subunit of the first TFP, the TCR subunit of the second TFP, or both comprise an intracellular domain comprising a stimulatory domain selected from a functional signaling domain of 4-1BB and/or a functional signaling domain of CD3 zeta, or an amino acid sequence having at least one modification thereto.

In one embodiment, the first human or humanized antibody domain, the second human or humanized antibody domain, or both comprise an antibody fragment. In another embodiment, the first human or humanized antibody domain, the second human or humanized antibody domain, or both comprise a scFv or a V_{H} domain. In another embodiment, the composition comprises a recombinant nucleic acid molecule encoding (i) a light chain (LC) CDR1, LC CDR2 and LC CDR3 of a light chain binding domain amino acid sequence with 70-100% sequence identity to a light chain sequence of Table 2, and/or (ii) a heavy chain (HC) CDR1, HC CDR2 and HC CDR3 of a heavy chain sequence of Table 2. In one embodiment, the recombinant nucleic acid encodes a light chain variable region, wherein the light chain variable region comprises an amino acid sequence having at least one but not more than 30 modifications of a light chain variable region amino acid sequence of Table 2, or a sequence with 95-99% identity to a light chain variable region amino acid sequence of Table 2. In another embodiment, the composition comprises a recombinant nucleic acid molecule encoding a heavy chain variable region, wherein the heavy chain variable region comprises an amino acid sequence having at least one but not more than 30 modifications of a heavy chain variable region amino acid sequence of Table 2, or a sequence with 95-99% identity to a heavy chain variable region amino acid sequence of Table 2. In one embodiment, the encoded first TFP, the encoded second TFP, or both include an extracellular domain of a TCR subunit that comprises an extracellular domain or portion thereof of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications. In another embodiment, the encoded first TFP and the encoded second TFP include a transmembrane domain that comprises a transmembrane domain of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications.

In one embodiment, the encoded first TFP and the encoded second TFP include a transmembrane domain that comprises a transmembrane domain of a protein selected from the group consisting of a TCR alpha chain, a TCR beta chain, a TCR zeta chain, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications. In another embodiment, the recombinant nucleic acid comprises a sequence encoding a costimulatory domain. In another embodiment, the costimulatory domain is a functional signaling domain obtained from a protein selected from the group consisting of OX40, CD2, CD27, CD28, CDS, ICAM-1, LFA-1 (CD11a/CD18), ICOS (CD278), and 4-1BB (CD137), and amino acid sequences thereof having at least one but not more than 20 modifications thereto. In another embodiment, the recombinant nucleic acid comprises a sequence encoding an intracellular signaling domain. In another embodiment, the recombinant nucleic acid comprises a sequence encoding a leader sequence. In another embodiment, the recombinant nucleic acid comprises a sequence encoding a protease cleavage site. In one embodiment the at least one but not more than 20 modifications thereto comprise a modification of an amino acid that mediates cell signaling or a modification of an amino acid that is phosphorylated in response to a ligand binding to the first TFP, the second TFP, or both.

In one embodiment, the isolated recombinant nucleic acid molecule is an mRNA.

In one embodiment, the first TFP, the second TFP, or both include an immunoreceptor tyrosine-based activation motif (ITAM) of a TCR subunit that comprises an ITAM or portion thereof of a protein selected from the group consisting of CD3 zeta TCR subunit, CD3 epsilon TCR subunit, CD3 gamma TCR subunit, CD3 delta TCR subunit, TCR zeta chain, Fc epsilon receptor 1 chain, Fc epsilon receptor 2 chain, Fc gamma receptor 1 chain, Fc gamma receptor 2a chain, Fc gamma receptor 2b1 chain, Fc gamma receptor 2b2 chain, Fc gamma receptor 3a chain, Fc gamma receptor 3b chain, Fe beta receptor 1 chain, TYROBP (DAP12), CD5, CD16a, CD16b, CD22, CD23, CD32, CD64, CD79a, CD79b, CD89, CD278, CD66d, functional fragments thereof, and amino acid sequences thereof having at least one but not more than 20 modifications thereto.

In another embodiment, the ITAM replaces an ITAM of CD3 gamma, CD3 delta, or CD3 epsilon. In another embodiment, the ITAM is selected from the group consisting of CD3 zeta TCR subunit, CD3 epsilon TCR subunit, CD3 gamma TCR subunit, and CD3 delta TCR subunit and replaces a different ITAM selected from the group consisting of CD3 zeta TCR subunit, CD3 epsilon TCR subunit, CD3 gamma TCR subunit, and CD3 delta TCR subunit. In one embodiment, the encoded recombinant nucleic acid further comprising a leader sequence.

In another aspect is provided a composition comprising a polypeptide molecule encoded by any of the nucleic acid molecules described herein. In one embodiment, the polypeptide comprises a first polypeptide encoded by a first nucleic acid molecule and a second polypeptide encoded by a second nucleic acid molecule.

In another aspect is provided a composition comprising a recombinant TFP molecule encoded by any of the nucleic acid molecules described herein.

In another aspect is provided a composition comprising a vector encoding the polypeptide or recombinant TFP molecule described herein. In one embodiment, the vector comprises a) a first vector comprising a first nucleic acid molecule encoding the first TFP; and b) a second vector comprising a second nucleic acid molecule encoding the second TFP. In another embodiment, the vector comprises a first TFP and a second TFP, wherein the sequence encoding the first TFP and the sequence encoding the second TFP are separated by a cleavage site. the vector is selected from the group consisting of a DNA, an RNA, a plasmid, a lentivirus vector, adenoviral vector, a Rous sarcoma viral (RSV) vector, or a retrovirus vector. In one embodiment, the vector comprises a promoter. In one embodiment, the vector is an in vitro transcribed vector. In one embodiment, the nucleic acid molecule in the vector further encodes a poly(A) tail. In another embodiment, the nucleic acid molecule in the vector further encodes a 3′ UTR. In another embodiment, the nucleic acid molecule in the vector further encodes a protease cleavage site.

In one embodiment, the composition further comprises a nucleic acid encoding an inhibitory molecule that comprises a first polypeptide that comprises at least a portion of an inhibitory molecule, associated with a second polypeptide that comprises a positive signal from an intracellular signaling domain. In another embodiment, the inhibitory molecule comprises a first polypeptide that comprises at least a portion of PD1 and a second polypeptide comprising a costimulatory domain and primary signaling domain.

In another aspect is provided a vector comprising the recombinant nucleic acid sequence disclosed herein. In one embodiment, the vector comprises the first recombinant nucleic acid sequence. In another embodiment, the vector comprises the second recombinant nucleic acid sequence.

In another aspect is provided a cell comprising a composition comprising any of the isolated recombinant nucleic acid molecules, vectors, or polypeptide disclosed herein. In one embodiment, the cell is a human T cell. In another embodiment, the T cell is a CD8+ or CD4+ T cell. In one embodiment, the cell comprises a nucleic acid encoding an inhibitory molecule that comprises a first polypeptide that comprises at least a portion of an inhibitory molecule, associated with a second polypeptide that comprises a positive signal from an intracellular signaling domain. In one embodiment, the inhibitory molecule comprises a first polypeptide that comprises at least a portion of PD1 and a second polypeptide comprising a costimulatory domain and primary signaling domain.

In another aspect is provided a human CD8+ or CD4+ T cell comprising at least two TFP molecules, the TFP molecules comprising an anti-MUC16 binding domain, an anti-MSLN binding domain, a TCR extracellular domain, a transmembrane domain, and an intracellular domain, wherein the TFP molecule is capable of functionally interacting with an endogenous TCR complex and/or at least one endogenous TCR polypeptide in, at and/or on the surface of the human CD8+ or CD4+ T cell. In another embodiment is a protein complex comprising a first TFP molecule comprising an anti-MUC16 binding domain, a TCR extracellular domain, a transmembrane domain, and an intracellular domain; a second TFP molecule comprising an anti-MSLN binding domain, a TCR extracellular domain, a transmembrane domain, and an intracellular domain; and at least one endogenous TCR subunit or endogenous TCR complex.

In another aspect is provided a protein complex comprising a TFP molecule comprising an anti-MUC16 binding domain, a TCR extracellular domain, a transmembrane domain, and an intracellular domain; and at least one endogenous TCR subunit or endogenous TCR complex.

In another aspect is provided a protein complex comprising a TFP molecule comprising an anti-MSLN binding domain, a TCR extracellular domain, a transmembrane domain, and an intracellular domain; and at least one endogenous TCR subunit or endogenous TCR complex.

In one embodiment, the TCR in the protein complex comprises an extracellular domain or portion thereof of a protein selected from the group consisting of TCR alpha chain, a TCR beta chain, a CD3 epsilon TCR subunit, a CD3 gamma TCR subunit, and a CD3 delta TCR subunit. In one embodiment, the anti-MUC16 binding domain, the anti-MSLN binding domain, or both are connected to the TCR extracellular domain by a linker sequence. In one embodiment, the linker region comprises (G₄S)ₙ, wherein n=1 to 4.

In another aspect is provided a human CD8+ or CD4+ T cell comprising at least two different TFP proteins in any of the protein complexes described herein. In another aspect is provided a human CD8+ or CD4+ T cell comprising at least two different TFP molecules encoded by any of the isolated nucleic acid molecules disclosed herein.

In another aspect is provided a population of human CD8+ or CD4+ T cells, wherein the T cells of the population individually or collectively comprise at least two TFP molecules, the TFP molecules comprising an anti-MUC16 binding domain or an anti-MSLN binding domain, or both an anti-MUC16 and an anti-MSLN binding domain, a TCR extracellular domain, a transmembrane domain, and an intracellular domain, wherein the TFP molecule is capable of functionally interacting with an endogenous TCR complex and/or at least one endogenous TCR polypeptide in, at and/or on the surface of the human CD8+ or CD4+ T cell.

In another aspect is provided a population of human CD8+ or CD4+ T cells, wherein the T cells of the population individually or collectively comprise at least two TFP molecules encoded by any of the isolated recombinant nucleic acid molecules disclosed herein. In another aspect is provided a pharmaceutical composition comprising an effective amount of a composition, vector, cell or protein complex disclosed herein, and a pharmaceutically acceptable excipient.

In another aspect is provided a method of treating a mammal having a disease associated with expression of MSLN or MUC16 comprising administering to the mammal an effective amount of any of the compositions disclosed herein. In one embodiment, the disease associated with MUC16 or MSLN expression is selected from the group consisting of a proliferative disease, a cancer, a malignancy, myelodysplasia, a myelodysplastic syndrome, a preleukemia, a non-cancer related indication associated with expression of MUC16, a non-cancer related indication associated with expression of MSLN, breast cancer, prostate cancer, ovarian cancer, cervical cancer, skin cancer, pancreatic cancer, colorectal cancer, renal cancer, liver cancer, brain cancer, lymphoma, leukemia, lung cancer, esophageal cancer, gastric cancer and unresectable ovarian cancer with relapsed or refractory disease. In another embodiment, the disease is a hematologic cancer selected from the group consisting of B-cell acute lymphoid leukemia (B-ALL), T cell acute lymphoid leukemia (T-ALL), acute lymphoblastic leukemia (ALL); chronic myelogenous leukemia (CML), chronic lymphocytic leukemia (CLL), B cell prolymphocytic leukemia, blastic plasmacytoid dendritic cell neoplasm, Burkitt's lymphoma, diffuse large B cell lymphoma, follicular lymphoma, hairy cell leukemia, small cell-follicular lymphoma, large cell-follicular lymphoma, malignant lymphoproliferative conditions, MALT lymphoma, mantle cell lymphoma, Marginal zone lymphoma, multiple myeloma, myelodysplasia, myelodysplastic syndrome, non-Hodgkin's lymphoma, plasmablastic lymphoma, plasmacytoid dendritic cell neoplasm, Waldenstrom macroglobulinemia, preleukemia, a disease associated with MUC16 or MSLN expression, and combinations thereof. In another embodiment, the cell or population of cells expressing a first TFP molecule and a second TFP molecule are administered in combination with an agent that increases the efficacy of a cell or population of cells expressing the first TFP molecule and the second TFP molecule. In one embodiment, less cytokines are released in the mammal compared a mammal administered an effective amount of a T cell expressing an anti-MSLN chimeric antigen receptor (CAR), an anti-MUC16 CAR, an anti-MSLN CAR and an anti-MUC16 CAR; or a combination thereof. In one embodiment, the cells expressing the first TFP molecule and a second TFP molecule are administered in combination with an agent that ameliorates one or more side effects associated with administration of a cell expressing the first TFP molecule and the second TFP molecule. In another embodiment, the first TFP molecule and a second TFP molecule are administered in combination with an agent that treats the disease associated with MSLN or MUC16.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 is a drawing showing some of the methods of dual targeting of cancer cells disclosed herein. Tumor cell antigen targets MUC16 and MSLN are exemplary antigens.

FIG. 2 depicts protein sequences showing the binding epitope on the MUC16 ectodomain sequence of anti-MUC16 antibodies R3MU4 and R3MU29 in comparison with the reported epitope of another antibody, 4H11.

FIG. 3 is a series of images from FACS analysis of Jurkat cells that were non-transduced (FIG. 3A, “NT”), transduced with an anti-mesothelin TFP (FIG. 3B, “MSLN TFP”), transduced with an anti-MUC16 TFP (FIG. 3C, “MUC16 TFP”) or a dual specific TFP (FIG. 3D). All Jurkat cells (NT, MSLN TFP, MUC16 TFP, dual specific TFP) were stained first with labelled Fc_MSLN and MUC16-biotin, concurrently, then stained with streptavidin-PE.

FIG. 4 is a graph showing measurement of IL-2 production by Jurkat cells that were non-transduced or transduced with MSLN TFPs, MUC16 TFPs or dual-specific TFPs and co-cultured with K562 cells (“DN”, circles), K562 cells expressing MSLN (“MSLN+”, squares), K562 cells expressing MUC16 (“MUC16+”, upward arrows), and K562 expressing both proteins (“DP”, downward arrows).

FIG. 5 is a series of images from FACS analysis of primary human T cells transduced with various constructs. NT (non-transduced), MSLN TFP, MUC16 TFP and dual-specific TFP T cells were generated from healthy donor T cells by transduction with a lentivirus encoding mono or dual-specific TFPs. Cells were expanded and stained as described for FIG. 3. Expression of MSLN specific TFPs (FIG. 5C), but not MUC16 TFPs (FIG. 5D), were detected for MSLN TFP T cells; in addition, MUC16 TFPs (FIG. 5F), but not MSLN TFPs (FIG. 5E), were detected for MUC16 TFP T cells. For dual-specific TFP T cells, both MSLN TFPs and MUC16 TFPs were detected on the surface of the transduced cells (FIGS. 5G and 5H). No detection of MSLN TFP or MUC16 TFP was observed for NT Jurkat cells (FIGS. 5A and 5B).

FIG. 6 is a graph showing measurement of cytotoxicity (as percentage of total) by primary human T cells cells that were non-transduced or transduced with MSLN TFPs, MUC16 TFPs or dual-specific TFPs and were co-cultured with K562 cells (“DN”, circles), K562 cells expressing MSLN (“MSLN+”, squares), K562 cells expressing MUC16 (“MUC16+”, upward arrows), and K562 expressing both proteins (“DP”, downward arrows).

FIG. 7A-C is a series of graphs showing target-specific cytokine production by primary human T cells that were non-transduced or transduced with MSLN TFPs, MUC16 TFPs or dual-specific TFPs and were co-cultured with K562 cells (“DN”, circles), K562 cells expressing MSLN (“MSLN+”, squares), K562 cells expressing MUC16 (“MUC16+”, upward arrows), and K562 expressing both proteins (“DP”, downward arrows) Cytokines measured were IFN-γ (FIG. 7A), GM-CSF (FIG. 7B), and TNF-α (FIG. 7C).

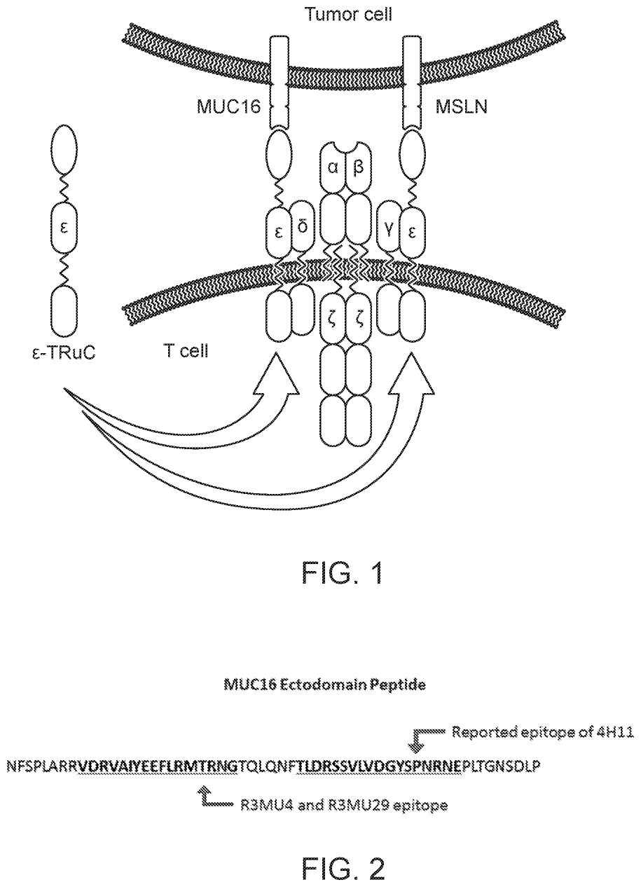

### DETAILED DESCRIPTION

Provided herein are compositions of matter and methods of use for the treatment of a disease such as cancer, using dual specificity T cell receptor (TCR) fusion proteins or dual specificity T cell populations. As used herein, a “T cell receptor (TCR) fusion protein” or “TFP” includes a recombinant polypeptide derived from the various polypeptides comprising the TCR that is generally capable of i) binding to a surface antigen on target cells and ii) interacting with other polypeptide components of the intact TCR complex, typically when co-located in or on the surface of a T cell. As provided herein, TFPs provide substantial benefits as compared to Chimeric Antigen Receptors. The term “Chimeric Antigen Receptor” or alternatively a “CAR” refers to a recombinant polypeptide comprising an extracellular antigen binding domain in the form of a scFv, a transmembrane domain, and cytoplasmic signaling domains (also referred to herein as “an intracellular signaling domains”) comprising a functional signaling domain derived from a stimulatory molecule as defined below. Generally, the central intracellular signaling domain of a CAR is derived from the CD3 zeta chain that is normally found associated with the TCR complex. The CD3 zeta signaling domain can be fused with one or more functional signaling domains derived from at least one co-stimulatory molecule such as 4-1BB (i.e., CD137), CD27 and/or CD28.

In one aspect, provided herein is a composition comprising (I) a first recombinant nucleic acid sequence encoding a first T cell receptor (TCR) fusion protein (TFP) comprising (a) a TCR subunit comprising (i) at least a portion of a TCR extracellular domain, (ii) a transmembrane domain, and (iii) a TCR intracellular domain comprising a stimulatory domain from an intracellular signaling domain derived only from a TCR subunit selected from the group consisting of a TCR alpha chain, a TCR beta chain, a CD3 gamma chain, a CD3 delta chain and a CD3 epsilon chain; and (b) a human or humanized antibody domain comprising an anti-MUC16 binding domain, wherein the TCR subunit and the anti-MUC16 binding domain are operatively linked, wherein the first TFP functionally interacts with a TCR or incorporate into a TCR when expressed in the T cell; and (II) a second recombinant nucleic acid sequence encoding a second TFP comprising (a) a TCR subunit comprising (i) at least a portion of a TCR extracellular domain, (ii) a transmembrane domain, and (iii) a TCR intracellular domain comprising a stimulatory domain from an intracellular signaling domain derived only from a TCR subunit selected from the group consisting of a TCR alpha chain, a TCR beta chain, a CD3 gamma chain, a CD3 delta chain and a CD3 epsilon chain; and (b) a human or humanized antibody domain comprising an anti-mesothelin (MSLN) binding domain, wherein the TCR subunit and the anti-MSLN binding domain are operatively linked, wherein the second TFP functionally interacts with a TCR or incorporates into a TCR when expressed in a T cell.

In one aspect, provided herein is a composition comprising (I) a first recombinant nucleic acid sequence encoding a first T cell receptor (TCR) fusion protein (TFP) comprising a TCR subunit comprising at least a portion of a TCR extracellular domain, a transmembrane domain, and a TCR intracellular domain comprising a stimulatory domain from an intracellular signaling domain derived only from a TCR subunit selected from the group consisting of a TCR alpha chain, a TCR beta chain, a CD3 gamma chain, a CD3 delta chain and a CD3 epsilon chain; and a first human or humanized antibody domain comprising an anti-MUC16 binding domain and a second human or humanized antibody domain comprising an anti-MSLN binding domain; wherein the TCR subunit, the first antibody domain, and the second antibody domain are operatively linked, and wherein the first TFP functionally interacts with a TCR or incorporates into a TCR when expressed in a T cell.

In one aspect, provided herein is a composition comprising a recombinant nucleic acid molecule encoding: a first T cell receptor (TCR) fusion protein (TFP) comprising a TCR subunit, a first human or humanized antibody domain comprising a first antigen binding domain that is an anti-MUC16 binding domain; and a second T cell receptor (TCR) fusion protein (TFP) comprising a TCR subunit, a second human or humanized antibody domain comprising a second antigen binding domain that is an anti-MSLN binding domain, wherein the TCR subunit of the first TFP and the first antibody domain are operatively linked and the TCR subunit of the second TFP and the second antibody domain are operatively linked.

In one aspect, provided herein is a composition comprising a recombinant nucleic acid molecule encoding: a first T cell receptor (TCR) fusion protein (TFP) comprising a TCR subunit, a first human or humanized antibody domain comprising a first antigen binding domain that is an anti-MUC16 binding domain and a second human or humanized antibody domain comprising a second antigen binding domain that is an anti-MSLN binding domain; and wherein the TCR subunit of the first TFP, the first antibody domain and the second antibody domain are operatively linked.

In some embodiments, the extracellular, transmembrane, and intracellular signaling domains of the TCR subunit of the first TFP are derived only from a TCR subunit selected from the group consisting of a TCR alpha chain, a TCR beta chain, a CD3 gamma chain, a CD3 delta chain and a CD3 epsilon chain.

In some embodiments, the extracellular, transmembrane, and intracellular signaling domains of the TCR subunit of the second TFP are derived only from a TCR subunit selected from the group consisting of a TCR alpha chain, a TCR beta chain, a TCR gamma chain, a TCR delta chain and a TCR epsilon chain.

In some embodiments, the extracellular, transmembrane, and intracellular signaling domains of the TCR subunit of the first TFP are derived only from a TCR alpha chain.

In some embodiments, the extracellular, transmembrane, and intracellular signaling domains of the TCR subunit of the first TFP are derived only from a TCR beta chain.

In some embodiments, the extracellular, transmembrane, and intracellular signaling domains of the TCR subunit of the first TFP are derived only from a TCR gamma chain.

In some embodiments, the extracellular, transmembrane, and intracellular signaling domains of the TCR subunit of the first TFP are derived only from a TCR delta chain.

In some embodiments, the extracellular, transmembrane, and intracellular signaling domains of the TCR subunit of the first TFP are derived only from a CD3 gamma chain.

In some embodiments, the extracellular, transmembrane, and intracellular signaling domains of the TCR subunit of the first TFP are derived only from a CD3 delta chain.

In some embodiments, the extracellular, transmembrane, and intracellular signaling domains of the TCR subunit of the first TFP are derived only from a CD3 epsilon chain.

In some embodiments, the extracellular, transmembrane, and intracellular signaling domains of the TCR subunit of the second TFP are derived only from a TCR alpha chain.

In some embodiments, the extracellular, transmembrane, and intracellular signaling domains of the TCR subunit of the second TFP are derived only from a TCR beta chain.

In some embodiments, the extracellular, transmembrane, and intracellular signaling domains of the TCR subunit of the second TFP are derived only from a TCR gamma chain.

In some embodiments, the extracellular, transmembrane, and intracellular signaling domains of the TCR subunit of the second TFP are derived only from a TCR delta chain.

In some embodiments, the extracellular, transmembrane, and intracellular signaling domains of the TCR subunit of the second TFP are derived only from a CD3 gamma chain.

In some embodiments, the extracellular, transmembrane, and intracellular signaling domains of the TCR subunit of the second TFP are derived only from a CD3 delta chain.

In some embodiments, the extracellular, transmembrane, and intracellular signaling domains of the TCR subunit of the second TFP are derived only from a CD3 epsilon chain.

In some embodiments, the first TFP, the second TFP, or both incorporate into a TCR or functionally interact with a TCR when expressed in a T cell.

In some embodiments, the first TFP, the second TFP, or both incorporate into a TCR or functionally interact with a TCR when expressed in a T cell.

In some embodiments, the encoded first antigen binding domain is connected to the TCR extracellular domain of the first TFP by a first linker sequence, the encoded second antigen binding domain is connected to the TCR extracellular domain of the second TFP by a second linker sequence, or both the first antigen binding domain is connected to the TCR extracellular domain of the first TFP by the first linker sequence and the encoded second antigen binding domain is connected to the TCR extracellular domain of the second TFP by the second linker sequence.

In some embodiments, the first linker sequence and the second linker sequence comprise (G4S)n, wherein n=1 to 4.

In some embodiments, the TCR subunit of the first TFP, the TCR subunit of the second TFP, or both comprise a TCR extracellular domain.

In some embodiments, the TCR subunit of the first TFP, the TCR subunit of the second TFP, or both comprise a TCR transmembrane domain.

In some embodiments, the TCR subunit of the first TFP, the TCR subunit of the second TFP, or both comprise a TCR intracellular domain.

In some embodiments, the TCR subunit of the first TFP, the TCR subunit of the second TFP, or both comprise (i) a TCR extracellular domain, (ii) a TCR transmembrane domain, and (iii) a TCR intracellular domain, wherein at least two of (i), (ii), and (iii) are from the same TCR subunit.

In some embodiments, the TCR subunit of the first TFP, the TCR subunit of the second TFP, or both comprise a TCR intracellular domain comprising a stimulatory domain selected from an intracellular signaling domain of CD3 epsilon, CD3 gamma or CD3 delta, or an amino acid sequence having at least one modification thereto.

In some embodiments, the TCR subunit of the first TFP, the TCR subunit of the second TFP, or both comprise an intracellular domain comprising a stimulatory domain selected from a functional signaling domain of 4-1BB and/or a functional signaling domain of CD3 zeta, or an amino acid sequence having at least one modification thereto.

In some embodiments, the first human or humanized antibody domain, the second human or humanized antibody domain, or both comprise an antibody fragment.

In some embodiments, the first human or humanized antibody domain, the second human or humanized antibody domain, or both comprise a scFv or a VH domain.

In some embodiments, the composition encodes (i) a light chain (LC) CDR1, LC CDR2 and LC CDR3 of a light chain binding domain amino acid sequence with 70-100% sequence identity to a light chain sequence of Table 2, and/or (ii) a heavy chain (HC) CDR1, HC CDR2 and HC CDR3 of a heavy chain sequence of Table 2.

In some embodiments, the composition encodes a light chain variable region, wherein the light chain variable region comprises an amino acid sequence having at least one but not more than 30 modifications of a light chain variable region amino acid sequence of Table 2, or a sequence with 95-99% identity to a light chain variable region amino acid sequence of Table 2.

In some embodiments, the composition encodes a heavy chain variable region, wherein the heavy chain variable region comprises an amino acid sequence having at least one but not more than 30 modifications of a heavy chain variable region amino acid sequence of Table 2, or a sequence with 95-99% identity to a heavy chain variable region amino acid sequence of Table 2.

In some embodiments, the encoded first TFP, the encoded second TFP, or both include an extracellular domain of a TCR subunit that comprises an extracellular domain or portion thereof of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications.

In some embodiments, the encoded first TFP and the encoded second TFP include a transmembrane domain that comprises a transmembrane domain of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications.

In some embodiments, the encoded first TFP and the encoded second TFP include a transmembrane domain that comprises a transmembrane domain of a protein selected from the group consisting of a TCR alpha chain, a TCR beta chain, a TCR zeta chain, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications.

In some embodiments, the composition further comprises a sequence encoding a costimulatory domain.

In some embodiments, the costimulatory domain is a functional signaling domain obtained from a protein selected from the group consisting of OX40, CD2, CD27, CD28, CDS, ICAM-1, LFA-1 (CD11a/CD18), ICOS (CD278), and 4-1BB (CD137), and amino acid sequences thereof having at least one but not more than 20 modifications thereto.

In some embodiments, the composition further comprises comprising a sequence encoding an intracellular signaling domain

In some embodiments, the composition further comprises a leader sequence.

In some embodiments, the composition further comprises a protease cleavage site.

In some embodiments, the at least one but not more than 20 modifications thereto comprise a modification of an amino acid that mediates cell signaling or a modification of an amino acid that is phosphorylated in response to a ligand binding to the first TFP, the second TFP, or both.

In some embodiments, the isolated nucleic acid molecule is an mRNA.

In some embodiments, the first TFP, the second TFP, or both include an immunoreceptor tyrosine-based activation motif (ITAM) of a TCR subunit that comprises an ITAM or portion thereof of a protein selected from the group consisting of CD3 zeta TCR subunit, CD3 epsilon TCR subunit, CD3 gamma TCR subunit, CD3 delta TCR subunit, TCR zeta chain, Fc epsilon receptor 1 chain, Fc epsilon receptor 2 chain, Fc gamma receptor 1 chain, Fc gamma receptor 2a chain, Fc gamma receptor 2b1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79a, CD79b, CD89, CD278, CD66d, functional fragments thereof, and amino acid sequences thereof having at least one but not more than 20 modifications thereto.

In some embodiments, the ITAM replaces an ITAM of CD3 gamma, CD3 delta, or CD3 epsilon.

In some embodiments, the ITAM is selected from the group consisting of CD3 zeta TCR subunit, CD3 epsilon TCR subunit, CD3 gamma TCR subunit, and CD3 delta TCR subunit and replaces a different ITAM selected from the group consisting of CD3 zeta TCR subunit, CD3 epsilon TCR subunit, CD3 gamma TCR subunit, and CD3 delta TCR subunit.

In some embodiments, the composition further comprises a leader sequence.

In one aspect, provided herein is a composition comprising a polypeptide molecule encoded by the nucleic acid molecule of a composition described herein.

In some embodiments, the polypeptide comprises a first polypeptide encoded by a first nucleic acid molecule and a second polypeptide encoded by a second nucleic acid molecule.

In one aspect, provided herein is a composition comprising a recombinant TFP molecule encoded by the nucleic acid molecule of a composition described herein.

In one aspect, provided herein is a composition comprising a vector comprising a nucleic acid molecule encoding a polypeptide or recombinant TFP molecule described herein.

In some embodiments, the vector comprises a) a first vector comprising a first nucleic acid molecule encoding the first TFP; and b) a second vector comprising a second nucleic acid molecule encoding the second TFP.

In some embodiments, the vector is selected from the group consisting of a DNA, an RNA, a plasmid, a lentivirus vector, adenoviral vector, a Rous sarcoma viral (RSV) vector, or a retrovirus vector.

In some embodiments, the vector further comprises a promoter.

In some embodiments, the vector is an in vitro transcribed vector.

In some embodiments, the nucleic acid molecule in the vector further encodes a poly(A) tail.

In some embodiments, the nucleic acid molecule in the vector further encodes a 3′UTR.

In some embodiments, the nucleic acid molecule in the vector further encodes a protease cleavage site.

In one aspect, provided herein is a composition comprising a cell comprising a composition described herein.

In some embodiments, the cell is a human T cell.

In some embodiments, the T cell is a CD8+ or CD4+ T cell.

In some embodiments, the composition further comprises a nucleic acid encoding an inhibitory molecule that comprises a first polypeptide that comprises at least a portion of an inhibitory molecule, associated with a second polypeptide that comprises a positive signal from an intracellular signaling domain.

In some embodiments, the inhibitory molecule comprises a first polypeptide that comprises at least a portion of PD1 and a second polypeptide comprising a costimulatory domain and primary signaling domain.

In one aspect, provided herein is a method of treating a mammal having a disease associated with expression of MSLN or MUC16 comprising administering to the mammal an effective amount of a composition described herein.

In some embodiments, the disease associated with MUC16 or MSLN, expression is selected from the group consisting of a proliferative disease, a cancer, a malignancy, myelodysplasia, a myelodysplastic syndrome, a preleukemia, a non-cancer related indication associated with expression of MUC16, a non-cancer related indication associated with expression of MSLN, breast cancer, prostate cancer, ovarian cancer, cervical cancer, skin cancer, pancreatic cancer, colorectal cancer, renal cancer, liver cancer, brain cancer, lymphoma, leukemia, lung cancer, esophageal cancer, gastric cancer and unresectable ovarian cancer with relapsed or refractory disease.

In some embodiments, the disease is a hematologic cancer selected from the group consisting of B-cell acute lymphoid leukemia (B-ALL), T cell acute lymphoid leukemia (T-ALL), acute lymphoblastic leukemia (ALL); chronic myelogenous leukemia (CML), chronic lymphocytic leukemia (CLL), B cell prolymphocytic leukemia, blastic plasmacytoid dendritic cell neoplasm, Burkitt's lymphoma, diffuse large B cell lymphoma, follicular lymphoma, hairy cell leukemia, small cell-follicular lymphoma, large cell-follicular lymphoma, malignant lymphoproliferative conditions, MALT lymphoma, mantle cell lymphoma, Marginal zone lymphoma, multiple myeloma, myelodysplasia, myelodysplastic syndrome, non-Hodgkin's lymphoma, plasmablastic lymphoma, plasmacytoid dendritic cell neoplasm, Waldenstrom macroglobulinemia, preleukemia, a disease associated with MUC16 or MSLN expression, and combinations thereof.

In some embodiments, the cells expressing a first TFP molecule and a second TFP molecule are administered in combination with an agent that increases the efficacy of a cell expressing the first TFP molecule and the second TFP molecule.

In some embodiments, less cytokines are released in the mammal compared a mammal administered an effective amount of a T cell expressing: an anti-MSLN chimeric antigen receptor (CAR); an anti-MUC16 CAR; an anti-MSLN CAR and an anti-MUC16 CAR; or a combination thereof.

In some embodiments, the cells expressing the first TFP molecule and a second TFP molecule are administered in combination with an agent that ameliorates one or more side effects associated with administration of a cell expressing the first TFP molecule and the second TFP molecule.

In some embodiments, the cells expressing the first TFP molecule and a second TFP molecule are administered in combination with an agent that treats the disease associated with MSLN or MUC16.

In one aspect, described herein are isolated nucleic acid molecules encoding a T cell Receptor (TCR) fusion protein (TFP) that comprise a TCR subunit and a human or humanized antibody domain comprising an anti-tumor antigen binding domain, such as anti-BCMA, anti-CD19, anti CD20, anti-CD22, anti-MUC16, anti-MSLN, etc. In some embodiments, the TCR subunit comprises a TCR extracellular domain. In other embodiments, the TCR subunit comprises a TCR transmembrane domain. In yet other embodiments, the TCR subunit comprises a TCR intracellular domain. In further embodiments, the TCR subunit comprises (i) a TCR extracellular domain, (ii) a TCR transmembrane domain, and (iii) a TCR intracellular domain, wherein at least two of (i), (ii), and (iii) are from the same TCR subunit. In yet further embodiments, the TCR subunit comprises a TCR intracellular domain comprising a stimulatory domain selected from an intracellular signaling domain of CD3 epsilon, CD3 gamma or CD3 delta, or an amino acid sequence having at least one, two or three modifications thereto. In yet further embodiments, the TCR subunit comprises an intracellular domain comprising a stimulatory domain selected from a functional signaling domain of 4-1BB and/or a functional signaling domain of CD3 zeta, or an amino acid sequence having at least one, two or three modifications thereto.

In some embodiments, the human or humanized antibody domain comprises an antibody fragment. In some embodiments, the human or humanized antibody domain comprises a scFv or a V_{H} domain.

In some embodiments, the isolated nucleic acid molecules comprise (i) a light chain (LC) CDR1, LC CDR2 and LC CDR3 of any anti-tumor-associated antigen light chain binding domain amino acid sequence provided herein, and/or (ii) a heavy chain (HC) CDR1, HC CDR2 and HC CDR3 of any anti-tumor-associated antigen heavy chain binding domain amino acid sequence provided herein.

In some embodiments, the light chain variable region comprises an amino acid sequence having at least one, two or three modifications but not more than 30, 20 or 10 modifications of an amino acid sequence of a light chain variable region provided herein, or a sequence with 95-99% identity to an amino acid sequence provided herein. In other embodiments, the heavy chain variable region comprises an amino acid sequence having at least one, two or three modifications but not more than 30, 20 or 10 modifications of an amino acid sequence of a heavy chain variable region provided herein, or a sequence with 95-99% identity to an amino acid sequence provided herein.

In some embodiments, the TFP includes an extracellular domain of a TCR subunit that comprises an extracellular domain or portion thereof of a protein selected from the group consisting of the alpha or beta chain of the T cell receptor, CD3 delta, CD3 epsilon, or CD3 gamma, or a functional fragment thereof, or an amino acid sequence having at least one, two or three modifications but not more than 20, 10 or 5 modifications thereto. In other embodiments, the encoded TFP includes a transmembrane domain that comprises a transmembrane domain of a protein selected from the group consisting of the alpha, beta chain of the TCR or TCR subunits CD3 epsilon, CD3 gamma and CD3 delta, or a functional fragment thereof, or an amino acid sequence having at least one, two or three modifications but not more than 20, 10 or 5 modifications thereto.

In some embodiments, the encoded TFP includes a transmembrane domain that comprises a transmembrane domain of a protein selected from the group consisting of the alpha, beta or zeta chain of the TCR or CD3 epsilon, CD3 gamma and CD3 delta CD45, CD4, CD5, CD8, CD9, CD16, CD22, CD33, CD28, CD37, CD64, CD80, CD86, CD134, CD137 and CD154, or a functional fragment thereof, or an amino acid sequence having at least one, two or three modifications but not more than 20, 10 or 5 modifications thereto.

In some embodiments, the encoded anti-tumor-associated antigen binding domain is connected to the TCR extracellular domain by a linker sequence. In some instances, the encoded linker sequence comprises (G₄S)ₙ, wherein n=1 to 4. In some instances, the encoded linker sequence comprises (G₄S)ₙ, wherein n=2 to 4. In some instances, the encoded linker sequence comprises (G₄S)ₙ, wherein n=1 to 3.

In some embodiments, the isolated nucleic acid molecules further comprise a sequence encoding a costimulatory domain. In some instances, the costimulatory domain is a functional signaling domain obtained from a protein selected from the group consisting of OX40, CD2, CD27, CD28, CDS, ICAM-1, LFA-1 (CD11a/CD18), ICOS (CD278), and 4-1BB (CD137), or an amino acid sequence having at least one, two or three modifications but not more than 20, 10 or 5 modifications thereto.

In some embodiments, the isolated nucleic acid molecules further comprise a leader sequence.

Also provided herein are isolated polypeptide molecules encoded by any of the previously described nucleic acid molecules.

Also provided herein in another aspect, are isolated T cell receptor fusion protein (TFP) molecules that comprise a human or humanized anti-tumor-associated antigen binding domain, a TCR extracellular domain, a transmembrane domain, and an intracellular domain. In some embodiments, the isolated TFP molecules comprises an antibody or antibody fragment comprising a human or humanized anti-tumor-associated antigen binding domain, a TCR extracellular domain, a transmembrane domain, and an intracellular domain.

In some embodiments, the anti-tumor-associated antigen binding domain is a scFv or a V_{H} domain. In other embodiments, the anti-tumor-associated antigen binding domain comprises a light chain and a heavy chain of an amino acid sequence provided herein, or a functional fragment thereof, or an amino acid sequence having at least one, two or three modifications but not more than 30, 20 or 10 modifications of an amino acid sequence of a light chain variable region provided herein, or a sequence with 95-99% identity with an amino acid sequence provided herein. In some embodiments, the isolated TFP molecules comprise a TCR extracellular domain that comprises an extracellular domain or portion thereof of a protein selected from the group consisting of the alpha or beta chain of the T cell receptor, CD3 delta, CD3 epsilon, or CD3 gamma, or an amino acid sequence having at least one, two or three modifications but not more than 20, 10 or 5 modifications thereto.

In some embodiments, the anti-tumor-associated antigen binding domain is connected to the TCR extracellular domain by a linker sequence. In some instances, the linker region comprises (G₄S)ₙ, wherein n=1 to 4. In some instances, the linker sequence comprises (G₄S)ₙ, wherein n=2 to 4. In some instances, the linker sequence comprises (G₄S)ₙ, wherein n=1 to 3.

In some embodiments, the isolated TFP molecules further comprise a sequence encoding a costimulatory domain. In other embodiments, the isolated TFP molecules further comprise a sequence encoding an intracellular signaling domain. In yet other embodiments, the isolated TFP molecules further comprise a leader sequence.

Also provided herein are vectors that comprise a nucleic acid molecule encoding any of the previously described TFP molecules. In some embodiments, the vector is selected from the group consisting of a DNA, an RNA, a plasmid, a lentivirus vector, adenoviral vector, or a retrovirus vector. In some embodiments, the vector further comprises a promoter. In some embodiments, the vector is an in vitro transcribed vector. In some embodiments, a nucleic acid sequence in the vector further comprises a poly(A) tail. In some embodiments, a nucleic acid sequence in the vector further comprises a 3′UTR

Also provided herein are cells that comprise any of the described vectors. In some embodiments, the cell is a human T cell. In some embodiments, the cell is a CD8+ or CD4+ T cell. In other embodiments, the cells further comprise a nucleic acid encoding an inhibitory molecule that comprises a first polypeptide that comprises at least a portion of an inhibitory molecule, associated with a second polypeptide that comprises a positive signal from an intracellular signaling domain. In some instances, the inhibitory molecule comprises a first polypeptide that comprises at least a portion of PD1 and a second polypeptide comprising a costimulatory domain and primary signaling domain.

In another aspect, provided herein are isolated TFP molecules that comprise a human or humanized anti-tumor-associated antigen binding domain, a TCR extracellular domain, a transmembrane domain, and an intracellular signaling domain, wherein the TFP molecule is capable of functionally interacting with an endogenous TCR complex and/or at least one endogenous TCR polypeptide.

In another aspect, provided herein are isolated TFP molecules that comprise a human or humanized anti-tumor-associated antigen binding domain, a TCR extracellular domain, a transmembrane domain, and an intracellular signaling domain, wherein the TFP molecule is capable of functionally integrating into an endogenous TCR complex.

In another aspect, provided herein are human CD8+ or CD4+ T cells that comprise at least two TFP molecules, the TFP molecules comprising a human or humanized anti-tumor-associated antigen binding domain, a TCR extracellular domain, a transmembrane domain, and an intracellular domain, wherein the TFP molecule is capable of functionally interacting with an endogenous TCR complex and/or at least one endogenous TCR polypeptide in, at and/or on the surface of the human CD8+ or CD4+ T cell.

In another aspect, provided herein are protein complexes that comprise i) a TFP molecule comprising a human or humanized anti-tumor-associated antigen binding domain, a TCR extracellular domain, a transmembrane domain, and an intracellular domain; and ii) at least one endogenous TCR complex.

In some embodiments, the TCR comprises an extracellular domain or portion thereof of a protein selected from the group consisting of the alpha or beta chain of the T cell receptor, CD3 delta, CD3 epsilon, or CD3 gamma. In some embodiments, the anti-tumor-associated antigen binding domain is connected to the TCR extracellular domain by a linker sequence. In some instances, the linker region comprises (G₄S)ₙ, wherein n=1 to 4. In some instances, the linker sequence comprises (G₄S)ₙ, wherein n=2 to 4. In some instances, the linker sequence comprises (G₄S)ₙ, wherein n=1 to 3.

Also provided herein are human CD8+ or CD4+ T cells that comprise at least two different TFP proteins per any of the described protein complexes.

In another aspect, provided herein is a population of human CD8+ or CD4+ T cells, wherein the T cells of the population individually or collectively comprise at least two TFP molecules, the TFP molecules comprising a human or humanized anti-tumor-associated antigen binding domain, a TCR extracellular domain, a transmembrane domain, and an intracellular domain, wherein the TFP molecule is capable of functionally interacting with an endogenous TCR complex and/or at least one endogenous TCR polypeptide in, at and/or on the surface of the human CD8+ or CD4+ T cell.

In another aspect, provided herein is a population of human CD8+ or CD4+ T cells, wherein the T cells of the population individually or collectively comprise at least two TFP molecules encoded by an isolated nucleic acid molecule provided herein.

In another aspect, provided herein are methods of making a cell comprising transducing a T cell with any of the described vectors.

In another aspect, provided herein are methods of generating a population of RNA-engineered cells that comprise introducing an in vitro transcribed RNA or synthetic RNA into a cell, where the RNA comprises a nucleic acid encoding any of the described TFP molecules.

In another aspect, provided herein are methods of providing an anti-tumor immunity in a mammal that comprise administering to the mammal an effective amount of a cell expressing any of the described TFP molecules. In some embodiments, the cell is an autologous T cell. In some embodiments, the cell is an allogeneic T cell. In some embodiments, the mammal is a human.

In another aspect, provided herein are methods of treating a mammal having a disease associated with expression of tumor-associated antigen that comprise administering to the mammal an effective amount of the cell comprising any of the described TFP molecules. In some embodiments, the disease associated with tumor-associated antigen expression is selected from a proliferative disease such as a cancer or malignancy or a precancerous condition such as a myelodysplasia, a myelodysplastic syndrome or a preleukemia, or is a non-cancer related indication associated with expression of tumor-associated antigen. In some embodiments, the disease is a hematologic cancer selected from the group consisting of one or more acute leukemias including but not limited to B-cell acute lymphoid leukemia (“B-ALL”), T cell acute lymphoid leukemia (“T-ALL”), acute lymphoblastic leukemia (ALL); one or more chronic leukemias including but not limited to chronic myelogenous leukemia (CML), chronic lymphocytic leukemia (CLL); additional hematologic cancers or hematologic conditions including, but not limited to B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, smoldering multiple myeloma, solitary plasmacytoma, lymphoplasmacytic lymphoma, plasma cell leukemia, myelodysplasia and myelodysplastic syndrome, non-Hodgkin's lymphoma, plasmablastic lymphoma, plasmacytoid dendritic cell neoplasm, Waldenstrom's macroglobulinemia, and “preleukemia” which are a diverse collection of hematological conditions united by ineffective production (or dysplasia) of myeloid blood cells, and to disease associated with tumor-associated antigen expression include, but not limited to atypical and/or non-classical cancers, malignancies, precancerous conditions or proliferative diseases expressing tumor-associated antigen; and combinations thereof.

In some embodiments, the cells expressing any of the described TFP molecules are administered in combination with an agent that ameliorates one or more side effects associated with administration of a cell expressing a TFP molecule. In some embodiments, the cells expressing any of the described TFP molecules are administered in combination with an agent that treats the disease associated with tumor-associated antigen.

Also provided herein are any of the described isolated nucleic acid molecules, any of the described isolated polypeptide molecules, any of the described isolated TFPs, any of the described protein complexes, any of the described vectors or any of the described cells for use as a medicament.

### 1. Definitions

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

As used herein, “about” can mean plus or minus less than 1 or 1, 2, 3, 4, 5, 6, 7, 8, 9, 10, 11, 12, 13, 14, 15, 16, 17, 18, 19, 20, 25, 30, or greater than 30 percent, depending upon the situation and known or knowable by one skilled in the art.

As used herein the specification, “subject” or “subjects” or “individuals” may include, but are not limited to, mammals such as humans or non-human mammals, e.g., domesticated, agricultural or wild, animals, as well as birds, and aquatic animals. “Patients” are subjects suffering from or at risk of developing a disease, disorder or condition or otherwise in need of the compositions and methods provided herein.

As used herein, “treating” or “treatment” refers to any indicia of success in the treatment or amelioration of the disease or condition. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. As used herein, “treat or prevent” is sometimes used herein to refer to a method that results in some level of treatment or amelioration of the disease or condition, and contemplates a range of results directed to that end, including but not restricted to prevention of the condition entirely.

As used herein, “preventing” refers to the prevention of the disease or condition, e.g., tumor formation, in the patient. For example, if an individual at risk of developing a tumor or other form of cancer is treated with the methods of the present invention and does not later develop the tumor or other form of cancer, then the disease has been prevented, at least over a period of time, in that individual.

As used herein, a “therapeutically effective amount” is the amount of a composition or an active component thereof sufficient to provide a beneficial effect or to otherwise reduce a detrimental non-beneficial event to the individual to whom the composition is administered. By “therapeutically effective dose” herein is meant a dose that produces one or more desired or desirable (e.g., beneficial) effects for which it is administered, such administration occurring one or more times over a given period of time. The exact dose will depend on the purpose of the treatment, and will be ascertainable by one skilled in the art using known techniques (see, e.g. Lieberman, Pharmaceutical Dosage Forms (vols. 1-3, 1992); Lloyd, The Art, Science and Technology of Pharmaceutical Compounding (1999); and Pickar, Dosage Calculations (1999))

As used herein, a “T cell receptor (TCR) fusion protein” or “TFP” includes a recombinant polypeptide derived from the various polypeptides comprising the TCR that is generally capable of i) binding to a surface antigen on target cells and ii) interacting with other polypeptide components of the intact TCR complex, typically when co-located in or on the surface of a T cell.

The term “antibody,” as used herein, refers to a protein, or polypeptide sequences derived from an immunoglobulin molecule, which specifically binds to an antigen. Antibodies can be intact immunoglobulins of polyclonal or monoclonal origin, or fragments thereof and can be derived from natural or from recombinant sources.

The terms “antibody fragment” or “antibody binding domain” refer to at least one portion of an antibody, or recombinant variants thereof, that contains the antigen binding domain, i.e., an antigenic determining variable region of an intact antibody, that is sufficient to confer recognition and specific binding of the antibody fragment to a target, such as an antigen and its defined epitope. Examples of antibody fragments include, but are not limited to, Fab, Fab′, F(ab′)₂, and Fv fragments, single-chain (sc)Fv (“scFv”) antibody fragments, linear antibodies, single domain antibodies (abbreviated “sdAb”) (either V_{L} or V_{H}), camelid V_{HH} domains, and multi-specific antibodies formed from antibody fragments.

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single polypeptide chain, and wherein the scFv retains the specificity of the intact antibody from which it is derived.

“Heavy chain variable region” or “V_{H}” (or, in the case of single domain antibodies, e.g., nanobodies, “V_{HH}”) with regard to an antibody refers to the fragment of the heavy chain that contains three CDRs interposed between flanking stretches known as framework regions, these framework regions are generally more highly conserved than the CDRs and form a scaffold to support the CDRs.

Unless specified, as used herein an scFv may have the V_{L} and V_{H} regions in either order, e.g., with respect to the N-terminal and C-terminal ends of the polypeptide, the scFv may comprise V_{L}-linker-V_{H} or may comprise V_{H}-linker-V_{L}.

The portion of the TFP composition of the invention comprising an antibody or antibody fragment thereof may exist in a variety of forms where the antigen binding domain is expressed as part of a contiguous polypeptide chain including, for example, a single domain antibody fragment (sdAb) or heavy chain antibodies HCAb 242:423-426). In one aspect, the antigen binding domain of a TFP composition of the invention comprises an antibody fragment. In a further aspect, the TFP comprises an antibody fragment that comprises a scFv or a sdAb.

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (“κ”) and lambda (“λ”) light chains refer to the two major antibody light chain isotypes.

The term “recombinant antibody” refers to an antibody that is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” refers to a molecule that is capable of being bound specifically by an antibody, or otherwise provokes an immune response. This immune response may involve either antibody production, or the activation of specific immunologically-competent cells, or both.

The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

The term “anti-tumor effect” refers to a biological effect which can be manifested by various means, including but not limited to, e.g., a decrease in tumor volume, a decrease in the number of tumor cells, a decrease in the number of metastases, an increase in life expectancy, decrease in tumor cell proliferation, decrease in tumor cell survival, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

The term “autologous” refers to any material derived from the same individual to whom it is later to be re-introduced into the individual.

The term “allogeneic” refers to any material derived from a different animal of the same species or different patient as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “xenogeneic” refers to a graft derived from an animal of a different species.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer, esophageal cancer, gastric cancer, unresectable ovarian cancer with relapsed or refractory disease, and the like.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a TFP of the invention can be replaced with other amino acid residues from the same side chain family and the altered TFP can be tested using the functional assays described herein.

The term “stimulation” refers to a primary response induced by binding of a stimulatory domain or stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, and/or reorganization of cytoskeletal structures, and the like.

The term “stimulatory molecule” or “stimulatory domain” refers to a molecule or portion thereof expressed by a T cell that provides the primary cytoplasmic signaling sequence(s) that regulate primary activation of the TCR complex in a stimulatory way for at least some aspect of the T cell signaling pathway. In one aspect, the primary signal is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or “ITAM”. Examples of an ITAM containing primary cytoplasmic signaling sequence that is of particular use in the invention includes, but is not limited to, those derived from TCR zeta, FcR gamma, FcR beta, CD3 gamma, CD3 delta, CD3 epsilon, CD5, CD22, CD79a, CD79b, CD278 (also known as “ICOS”) and CD66d.

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T cells may recognize these complexes using their T cell receptors (TCRs). APCs process antigens and present them to T cells.

An “intracellular signaling domain,” as the term is used herein, refers to an intracellular portion of a molecule. The intracellular signaling domain generates a signal that promotes an immune effector function of the TFP containing cell, e.g., a TFP-expressing T cell. Examples of immune effector function, e.g., in a TFP-expressing T cell, include cytolytic activity and T helper cell activity, including the secretion of cytokines. In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation.

A primary intracellular signaling domain can comprise an ITAM (“immunoreceptor tyrosine-based activation motif”). Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, and CD66d DAP10 and DAP12.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to, an MHC class 1 molecule, BTLA and a Toll ligand receptor, as well as OX40, CD2, CD27, CD28, CDS, ICAM-1, LFA-1 (CD11a/CD18) and 4-1BB (CD137). A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, and a ligand that specifically binds with CD83, and the like. The intracellular signaling domain can comprise the entire intracellular portion, or the entire native intracellular signaling domain, of the molecule from which it is derived, or a functional fragment thereof. The term “4-1BB” refers to a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank Acc. No. AAA62478.2, or equivalent residues from non-human species, e.g., mouse, rodent, monkey, ape and the like.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain one or more introns.

The term “effective amount” or “therapeutically effective amount” are used interchangeably herein, and refer to an amount of a compound, formulation, material, or composition, as described herein effective to achieve a particular biological or therapeutic result.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic include, but are not limited to, e.g., the LENTIVECTOR™ gene delivery technology from Oxford BioMedica, the LENTIMAX™ vector system from Lentigen, and the like. Nonclinical types of lentiviral vectors are also available and would be known to one skilled in the art.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Human” or “fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “parenteral” administration of an immunogenic composition includes, e.g., subcutaneous (s.c.), intravenous (i.v.), intramuscular (i.m.), or intrasternal injection, intratumoral, or infusion techniques.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the transcription machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

The terms “linker” and “flexible polypeptide linker” as used in the context of a scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)ₙ, where n is a positive integer equal to or greater than 1. For example, n=1, n=2, n=3, n=4, n=5, n=6, n=7, n=8, n=9 and n=10. In one embodiment, the flexible polypeptide linkers include, but are not limited to, (Gly₄Ser)₄ or (Gly₄Ser)₃. In another embodiment, the linkers include multiple repeats of (Gly₂Ser), (GlySer) or (Gly₃Ser). Also included within the scope of the invention are linkers described in WO2012/138475 (incorporated herein by reference). In some instances, the linker sequence comprises (G₄S)ₙ, wherein n=2 to 4. In some instances, the linker sequence comprises (G₄S)ₙ, wherein n=1 to 3.

As used herein, a 5′ cap (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA, preferably mRNA, which has been synthesized in vitro. Generally, the in vitro transcribed RNA is generated from an in vitro transcription vector. The in vitro transcription vector comprises a template that is used to generate the in vitro transcribed RNA.

As used herein, a “poly(A)” is a series of adenosines attached by polyadenylation to the mRNA. In the preferred embodiment of a construct for transient expression, the polyA is between 50 and 5000, preferably greater than 64, more preferably greater than 100, most preferably greater than 300 or 400. Poly(A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA (SEQ ID NO:98) near the cleavage site. After the mRNA has been cleaved, adenosine residues are added to the free 3′ end at the cleavage site.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human).

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

In the context of the present invention, “tumor antigen” or “hyperproliferative disorder antigen” or “antigen associated with a hyperproliferative disorder” refers to antigens that are common to specific hyperproliferative disorders. In certain aspects, the hyperproliferative disorder antigens of the present invention are derived from, cancers including but not limited to primary or metastatic melanoma, thymoma, lymphoma, sarcoma, lung cancer, liver cancer, NHL, leukemias, uterine cancer, cervical cancer, bladder cancer, kidney cancer and adenocarcinomas such as breast cancer, prostate cancer, ovarian cancer, cervical cancer, skin cancer, pancreatic cancer, colorectal cancer, renal cancer, liver cancer, brain cancer, lymphoma, leukemia, lung cancer, esophageal cancer, gastric cancer, unresectable ovarian cancer with relapsed or refractory disease.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, an antibody fragment or a specific ligand, which recognizes and binds a cognate binding partner (e.g., BCMA) present in a sample, but which does not necessarily and substantially recognize or bind other molecules in the sample.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

### 2. T Cell Receptor (TCR) Fusion Proteins (TFP)

The present invention encompasses recombinant DNA constructs encoding TFPs, wherein the TFP comprises an antibody fragment that binds specifically to BCMA, e.g., human BCMA, wherein the sequence of the antibody fragment is contiguous with and in the same reading frame as a nucleic acid sequence encoding a TCR subunit or portion thereof. The TFPs provided herein are able to associate with one or more endogenous (or alternatively, one or more exogenous, or a combination of endogenous and exogenous) TCR subunits in order to form a functional TCR complex.

In one aspect, the TFP of the invention comprises a target-specific binding element otherwise referred to as an antigen binding domain. The choice of moiety depends upon the type and number of target antigen that define the surface of a target cell. For example, the antigen binding domain may be chosen to recognize a target antigen that acts as a cell surface marker on target cells associated with a particular disease state. Thus, examples of cell surface markers that may act as target antigens for the antigen binding domain in a TFP of the invention include those associated with viral, bacterial and parasitic infections; autoimmune diseases; and cancerous diseases (e.g., malignant diseases).

In one aspect, the TFP-mediated T cell response can be directed to an antigen of interest by way of engineering an antigen-binding domain into the TFP that specifically binds a desired antigen.

In one aspect, the portion of the TFP comprising the antigen binding domain comprises an antigen binding domain that targets BCMA. In one aspect, the antigen binding domain targets human BCMA.

The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (V_{H}), a light chain variable domain (V_{L}) and a variable domain (V_{HH}) of a camelid derived nanobody, and to an alternative scaffold known in the art to function as antigen binding domain, such as a recombinant fibronectin domain, anticalin, DARPIN and the like. Likewise, a natural or synthetic ligand specifically recognizing and binding the target antigen can be used as antigen binding domain for the TFP. In some instances, it is beneficial for the antigen binding domain to be derived from the same species in which the TFP will ultimately be used in. For example, for use in humans, it may be beneficial for the antigen binding domain of the TFP to comprise human or humanized residues for the antigen binding domain of an antibody or antibody fragment.

Thus, in one aspect, the antigen-binding domain comprises a humanized or human antibody or an antibody fragment, or a murine antibody or antibody fragment. In one embodiment, the humanized or human anti-BCMA binding domain comprises one or more (e.g., all three) light chain complementary determining region 1 (LC CDR1), light chain complementary determining region 2 (LC CDR2), and light chain complementary determining region 3 (LC CDR3) of a humanized or human anti-BCMA binding domain described herein, and/or one or more (e.g., all three) heavy chain complementary determining region 1 (HC CDR1), heavy chain complementary determining region 2 (HC CDR2), and heavy chain complementary determining region 3 (HC CDR3) of a humanized or human anti-BCMA binding domain described herein, e.g., a humanized or human anti-BCMA binding domain comprising one or more, e.g., all three, LC CDRs and one or more, e.g., all three, HC CDRs. In one embodiment, the humanized or human anti-BCMA binding domain comprises one or more (e.g., all three) heavy chain complementary determining region 1 (HC CDR1), heavy chain complementary determining region 2 (HC CDR2), and heavy chain complementary determining region 3 (HC CDR3) of a humanized or human anti-BCMA binding domain described herein, e.g., the humanized or human anti-tumor-associated antigen binding domain has two variable heavy chain regions, each comprising a HC CDR1, a HC CDR2 and a HC CDR3 described herein. In one embodiment, the humanized or human anti-tumor-associated antigen binding domain comprises a humanized or human light chain variable region described herein and/or a humanized or human heavy chain variable region described herein. In one embodiment, the humanized or human anti-tumor-associated antigen binding domain comprises a humanized heavy chain variable region described herein, e.g., at least two humanized or human heavy chain variable regions described herein. In one embodiment, the anti-tumor-associated antigen binding domain is a scFv comprising a light chain and a heavy chain of an amino acid sequence provided herein. In an embodiment, the anti-tumor-associated antigen binding domain (e.g., an scFv or V_{H}H nb) comprises: a light chain variable region comprising an amino acid sequence having at least one, two or three modifications (e.g., substitutions) but not more than 30, 20 or 10 modifications (e.g., substitutions) of an amino acid sequence of a light chain variable region provided herein, or a sequence with 95-99% identity with an amino acid sequence provided herein; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided herein, or a sequence with 95-99% identity to an amino acid sequence provided herein. In one embodiment, the humanized or human anti-tumor-associated antigen binding domain is a scFv, and a light chain variable region comprising an amino acid sequence described herein, is attached to a heavy chain variable region comprising an amino acid sequence described herein, via a linker, e.g., a linker described herein. In one embodiment, the humanized anti-tumor-associated antigen binding domain includes a (Gly₄-Ser)ₙ linker, wherein n is 1, 2, 3, 4, 5, or 6, preferably 3 or 4. The light chain variable region and heavy chain variable region of a scFv can be, e.g., in any of the following orientations: light chain variable region-linker-heavy chain variable region or heavy chain variable region-linker-light chain variable region. In some instances, the linker sequence comprises (G₄S)ₙ, wherein n=2 to 4. In some instances, the linker sequence comprises (G₄S)ₙ, wherein n=1 to 3.

In some aspects, a non-human antibody is humanized, where specific sequences or regions of the antibody are modified to increase similarity to an antibody naturally produced in a human or fragment thereof. In one aspect, the antigen binding domain is humanized.

A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein in its entirety by reference. Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions (see, e.g., Queen et al., U.S. Pat. No. 5,585,089; and Riechmann et al., 1988, Nature, 332:323, which are incorporated herein by reference in their entireties.)

A humanized antibody or antibody fragment has one or more amino acid residues remaining in it from a source which is nonhuman. These nonhuman amino acid residues are often referred to as “import” residues, which are typically taken from an “import” variable domain. As provided herein, humanized antibodies or antibody fragments comprise one or more CDRs from nonhuman immunoglobulin molecules and framework regions wherein the amino acid residues comprising the framework are derived completely or mostly from human germline. Multiple techniques for humanization of antibodies or antibody fragments are well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference in their entirety). In such humanized antibodies and antibody fragments, substantially less than an intact human variable domain has been substituted by the corresponding sequence from a nonhuman species. Humanized antibodies are often human antibodies in which some CDR residues and possibly some framework (FR) residues are substituted by residues from analogous sites in rodent antibodies. Humanization of antibodies and antibody fragments can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332), the contents of which are incorporated herein by reference in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (see, e.g., Nicholson et al. Mol. Immun. 34 (16-17): 1157-1165 (1997); Carter et al., Proc. Natl. Acad. Sci. USA, 89:4285 (1992); Presta et al., J. Immunol., 151:2623 (1993), the contents of which are incorporated herein by reference herein in their entirety). In some embodiments, the framework region, e.g., all four framework regions, of the heavy chain variable region are derived from a V_{H}4-4-59 germline sequence. In one embodiment, the framework region can comprise, one, two, three, four or five modifications, e.g., substitutions, e.g., from the amino acid at the corresponding murine sequence. In one embodiment, the framework region, e.g., all four framework regions of the light chain variable region are derived from a VK3-1.25 germline sequence. In one embodiment, the framework region can comprise, one, two, three, four or five modifications, e.g., substitutions, e.g., from the amino acid at the corresponding murine sequence.

In some aspects, the portion of a TFP composition of the invention that comprises an antibody fragment is humanized with retention of high affinity for the target antigen and other favorable biological properties. According to one aspect of the invention, humanized antibodies and antibody fragments are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, e.g., the analysis of residues that influence the ability of the candidate immunoglobulin to bind the target antigen. In this way, FR residues can be selected and combined from the recipient and import sequences so that the desired antibody or antibody fragment characteristic, such as increased affinity for the target antigen, is achieved. In general, the CDR residues are directly and most substantially involved in influencing antigen binding.

In one aspect, the anti-tumor-associated antigen binding domain is a fragment, e.g., a single chain variable fragment (scFv) or a camelid heavy chain (V_{H}H). In one aspect, the anti-tumor-associated antigen binding domain is a Fv, a Fab, a (Fab′)₂, or a bi-functional (e.g. bi-specific) hybrid antibody (e.g., Lanzavecchia et al., Eur. J. Immunol. 17, 105 (1987)). In one aspect, the antibodies and fragments thereof of the invention binds a tumor-associated antigen protein with wild-type or enhanced affinity.

Also provided herein are methods for obtaining an antibody antigen binding domain specific for a target antigen (e.g., BCMA or any target antigen described elsewhere herein for targets of fusion moiety binding domains), the method comprising providing by way of addition, deletion, substitution or insertion of one or more amino acids in the amino acid sequence of a V_{H} (or V_{H}H) domain set out herein a V_{H} domain which is an amino acid sequence variant of the V_{H} domain, optionally combining the V_{H} domain thus provided with one or more V_{L} domains, and testing the V_{H} domain or V_{H}/V_{L} combination or combinations to identify a specific binding member or an antibody antigen binding domain specific for a target antigen of interest (e.g., BCMA) and optionally with one or more desired properties.

In some instances, V_{H} domains and scFvs can be prepared according to method known in the art (see, for example, Bird et al., (1988) Science 242:423-426 and Huston et al., (1988) Proc. Natl. Acad. Sci. USA 85:5879-5883). scFv molecules can be produced by linking V_{H} and V_{L} regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intra-chain folding is prevented. Inter-chain folding is also required to bring the two variable regions together to form a functional epitope binding site. In some instances, the linker sequence comprises (G₄S)ₙ, wherein n=2 to 4. In some instances, the linker sequence comprises (G₄S)ₙ, wherein n=1 to 3. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

A scFv can comprise a linker of about 10, 11, 12, 13, 14, 15 or greater than 15 residues between its V_{L} and V_{H} regions. The linker sequence may comprise any naturally occurring amino acid. In some embodiments, the linker sequence comprises amino acids glycine and serine. In another embodiment, the linker sequence comprises sets of glycine and serine repeats such as (Gly₄Ser)ₙ, where n is a positive integer equal to or greater than 1. In one embodiment, the linker can be (Gly₄Ser)₄ or (Gly₄Ser)₃. Variation in the linker length may retain or enhance activity, giving rise to superior efficacy in activity studies. In some instances, the linker sequence comprises (G₄S)ₙ, wherein n=2 to 4. In some instances, the linker sequence comprises (G₄S)ₙ, wherein n=1 to 3.

### 3. Stability and Mutations

The stability of an anti-tumor-associated antigen binding domain, e.g., scFv molecules (e.g., soluble scFv) can be evaluated in reference to the biophysical properties (e.g., thermal stability) of a conventional control scFv molecule or a full-length antibody. In one embodiment, the humanized or human scFv has a thermal stability that is greater than about 0.1, about 0.25, about 0.5, about 0.75, about 1, about 1.25, about 1.5, about 1.75, about 2, about 2.5, about 3, about 3.5, about 4, about 4.5, about 5, about 5.5, about 6, about 6.5, about 7, about 7.5, about 8, about 8.5, about 9, about 9.5, about 10 degrees, about 11 degrees, about 12 degrees, about 13 degrees, about 14 degrees, or about 15 degrees Celsius than a parent scFv in the described assays.

The improved thermal stability of the anti-tumor-associated antigen binding domain, e.g., scFv is subsequently conferred to the entire tumor-associated antigen-TFP construct, leading to improved therapeutic properties of the anti-tumor-associated antigen TFP construct. The thermal stability of the anti-tumor-associated antigen binding domain, e.g., scFv can be improved by at least about 2° C. or 3° C. as compared to a conventional antibody. In one embodiment, the anti-tumor-associated antigen binding domain, e.g., scFv has a 1° C. improved thermal stability as compared to a conventional antibody. In another embodiment, the anti-tumor-associated antigen binding domain, e.g., scFv has a 2° C. improved thermal stability as compared to a conventional antibody. In another embodiment, the scFv has a 4° C., 5° C., 6° C., 7° C., 8° C., 9° C., 10° C., 11° C., 12° C., 13° C., 14° C., or 15° C. improved thermal stability as compared to a conventional antibody. Comparisons can be made, for example, between the scFv molecules disclosed herein and scFv molecules or Fab fragments of an antibody from which the scFv V_{H} and V_{L} were derived. Thermal stability can be measured using methods known in the art. For example, in one embodiment, T_{M} can be measured. Methods for measuring T_{M} and other methods of determining protein stability are described below.

Mutations in scFv (arising through humanization or mutagenesis of the soluble scFv) alter the stability of the scFv and improve the overall stability of the scFv and the anti-tumor-associated antigen TFP construct. Stability of the humanized scFv is compared against the murine scFv using measurements such as T_{M}, temperature denaturation and temperature aggregation. In one embodiment, the anti-tumor-associated antigen binding domain, e.g., a scFv, comprises at least one mutation arising from the humanization process such that the mutated scFv confers improved stability to the anti-tumor-associated antigen TFP construct. In another embodiment, the anti-tumor-associated antigen binding domain, e.g., scFv comprises at least 1, 2, 3, 4, 5, 6, 7, 8, 9, 10 mutations arising from the humanization process such that the mutated scFv confers improved stability to the tumor-associated antigen-TFP construct.

In one aspect, the antigen binding domain of the TFP comprises an amino acid sequence that is homologous to an antigen binding domain amino acid sequence described herein, and the antigen binding domain retains the desired functional properties of the anti-tumor-associated antigen antibody fragments described herein. In one specific aspect, the TFP composition of the invention comprises an antibody fragment. In a further aspect, that antibody fragment comprises a scFv.

In various aspects, the antigen binding domain of the TFP is engineered by modifying one or more amino acids within one or both variable regions (e.g., V_{H} and/or V_{L}), for example within one or more CDR regions and/or within one or more framework regions. In one specific aspect, the TFP composition of the invention comprises an antibody fragment. In a further aspect, that antibody fragment comprises a scFv.

It will be understood by one of ordinary skill in the art that the antibody or antibody fragment of the invention may further be modified such that they vary in amino acid sequence (e.g., from wild-type), but not in desired activity. For example, additional nucleotide substitutions leading to amino acid substitutions at “non-essential” amino acid residues may be made to the protein. For example, a nonessential amino acid residue in a molecule may be replaced with another amino acid residue from the same side chain family. In another embodiment, a string of amino acids can be replaced with a structurally similar string that differs in order and/or composition of side chain family members, e.g., a conservative substitution, in which an amino acid residue is replaced with an amino acid residue having a similar side chain, may be made.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Percent identity in the context of two or more nucleic acids or polypeptide sequences refers to two or more sequences that are the same. Two sequences are “substantially identical” if two sequences have a specified percentage of amino acid residues or nucleotides that are the same (e.g., 60% identity, optionally 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art. Optimal alignment of sequences for comparison can be conducted, e.g., by the local homology algorithm of Smith and Waterman, (1970) Adv. Appl. Math. 2:482c, by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., (2003) Current Protocols in Molecular Biology). Two examples of algorithms that are suitable for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

In one aspect, the present invention contemplates modifications of the starting antibody or fragment (e.g., scFv) amino acid sequence that generate functionally equivalent molecules. For example, the V_{H} or V_{L} of an anti-tumor-associated antigen binding domain, e.g., scFv, comprised in the TFP can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity of the starting V_{H} or V_{L} framework region of the anti-tumor-associated antigen binding domain, e.g., scFv. The present invention contemplates modifications of the entire TFP construct, e.g., modifications in one or more amino acid sequences of the various domains of the TFP construct in order to generate functionally equivalent molecules. The TFP construct can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity of the starting TFP construct.

### 4. Extracellular Domain

The extracellular domain may be derived either from a natural or from a recombinant source. Where the source is natural, the domain may be derived from any protein, but in particular a membrane-bound or transmembrane protein. In one aspect, the extracellular domain is capable of associating with the transmembrane domain. An extracellular domain of particular use in this invention may include at least the extracellular region(s) of e.g., the alpha, beta or zeta chain of the T cell receptor, or CD3 epsilon, CD3 gamma, or CD3 delta, or in alternative embodiments, CD28, CD45, CD4, CD5, CD8, CD9, CD16, CD22, CD33, CD37, CD64, CD80, CD86, CD134, CD137, CD154.

### 5. Transmembrane Domain

In general, a TFP sequence contains an extracellular domain and a transmembrane domain encoded by a single genomic sequence. In alternative embodiments, a TFP can be designed to comprise a transmembrane domain that is heterologous to the extracellular domain of the TFP. A transmembrane domain can include one or more additional amino acids adjacent to the transmembrane region, e.g., one or more amino acid associated with the extracellular region of the protein from which the transmembrane was derived (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids of the extracellular region) and/or one or more additional amino acids associated with the intracellular region of the protein from which the transmembrane protein is derived (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids of the intracellular region) In some cases, the transmembrane domain can include at least 30, 35, 40, 45, 50, 55, 60 or more amino acids of the extracellular region. In some cases, the transmembrane domain can include at least 30, 35, 40, 45, 50, 55, 60 or more amino acids of the intracellular region. In one aspect, the transmembrane domain is one that is associated with one of the other domains of the TFP is used. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another TFP on the TFP T cell surface. In a different aspect the amino acid sequence of the transmembrane domain may be modified or substituted so as to minimize interactions with the binding domains of the native binding partner present in the same TFP.

The transmembrane domain may be derived either from a natural or from a recombinant source. Where the source is natural, the domain may be derived from any membrane-bound or transmembrane protein. In one aspect, the transmembrane domain is capable of signaling to the intracellular domain(s) whenever the TFP has bound to a target. A transmembrane domain of particular use in this invention may include at least the transmembrane region(s) of e.g., the alpha, beta or zeta chain of the T cell receptor, CD28, CD3 epsilon, CD45, CD4, CD5, CD8, CD9, CD16, CD22, CD33, CD37, CD64, CD80, CD86, CD134, CD137, CD154.

In some instances, the transmembrane domain can be attached to the extracellular region of the TFP, e.g., the antigen binding domain of the TFP, via a hinge, e.g., a hinge from a human protein. For example, in one embodiment, the hinge can be a human immunoglobulin (Ig) hinge, e.g., an IgG4 hinge, or a CD8a hinge.

### 6. Linkers

Optionally, a short oligo- or polypeptide linker, between 2 and 10 amino acids in length may form the linkage between the transmembrane domain and the cytoplasmic region of the TFP. A glycine-serine doublet provides a particularly suitable linker. For example, in one aspect, the linker comprises the amino acid sequence of GGGGSGGGGS. In some embodiments, the linker is encoded by a nucleotide sequence of GGTGGCGGAGGTTCTGGAGGTGGAGGTTCC.

### 7. Cytoplasmic Domain

The cytoplasmic domain of the TFP can include an intracellular signaling domain, if the TFP contains CD3 gamma, delta or epsilon polypeptides; TCR alpha and TCR beta subunits are generally lacking in a signaling domain. An intracellular signaling domain is generally responsible for activation of at least one of the normal effector functions of the immune cell in which the TFP has been introduced. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Examples of intracellular signaling domains for use in the TFP of the invention include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of naive T cells and that a secondary and/or costimulatory signal is required. Thus, naïve T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs (ITAMs).

Examples of ITAMs containing primary intracellular signaling domains that are of particular use in the invention include those of CD3 zeta, FcR gamma, FcR beta, CD3 gamma, CD3 delta, CD3 epsilon, CD5, CD22, CD79a, CD79b, and CD66d. In one embodiment, a TFP of the invention comprises an intracellular signaling domain, e.g., a primary signaling domain of CD3-epsilon. In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs.

The intracellular signaling domain of the TFP can comprise the CD3 zeta signaling domain by itself or it can be combined with any other desired intracellular signaling domain(s) useful in the context of a TFP of the invention. For example, the intracellular signaling domain of the TFP can comprise a CD3 epsilon chain portion and a costimulatory signaling domain. The costimulatory signaling domain refers to a portion of the TFP comprising the intracellular domain of a costimulatory molecule. A costimulatory molecule is a cell surface molecule other than an antigen receptor or its ligands that is required for an efficient response of lymphocytes to an antigen. Examples of such molecules include CD27, CD28, 4-1BB (CD137), OX40, CD30, CD40, PD1, ICOS, lymphocyte function-associated antigen-1 (LFA-1), CD2, CD7, LIGHT, NKG2C, B7-H3, and a ligand that specifically binds with CD83, and the like. For example, CD27 costimulation has been demonstrated to enhance expansion, effector function, and survival of human TFP-T cells in vitro and augments human T cell persistence and antitumor activity in vivo (Song et al. Blood. 2012; 119(3):696-706).

The intracellular signaling sequences within the cytoplasmic portion of the TFP of the invention may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequences.

In one embodiment, a glycine-serine doublet can be used as a suitable linker. In one embodiment, a single amino acid, e.g., an alanine, a glycine, can be used as a suitable linker.

In one aspect, the TFP-expressing cell described herein can further comprise a second TFP, e.g., a second TFP that includes a different antigen binding domain, e.g., to the same target (e.g., MUC16 or MSLN,) or a different target (e.g., MUC16 or MSLN). In one embodiment, when the TFP-expressing cell comprises two or more different TFPs, the antigen binding domains of the different TFPs can be such that the antigen binding domains do not interact with one another. For example, a cell expressing a first and second TFP can have an antigen binding domain of the first TFP, e.g., as a fragment, e.g., a scFv, that does not associate with the antigen binding domain of the second TFP, e.g., the antigen binding domain of the second TFP is a V_{HH}.

In another aspect, the TFP-expressing cell described herein can further express another agent, e.g., an agent which enhances the activity of a TFP-expressing cell. For example, in one embodiment, the agent can be an agent which inhibits an inhibitory molecule. Inhibitory molecules, e.g., PD1, can, in some embodiments, decrease the ability of a TFP-expressing cell to mount an immune effector response. Examples of inhibitory molecules include PD1, PD-L1, CTLA4, TIM3, LAG3, VISTA, BTLA, TIGIT, LAIR1, CD160, 2B4 and TGFR beta. In one embodiment, the agent that inhibits an inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD1, LAG3, CTLA4, CD160, BTLA, LAIR1, TIM3, 2B4 and TIGIT, or a fragment of any of these (e.g., at least a portion of an extracellular domain of any of these), and a second polypeptide which is an intracellular signaling domain described herein (e.g., comprising a costimulatory domain (e.g., 4-1BB, CD27 or CD28, e.g., as described herein) and/or a primary signaling domain (e.g., a CD3 zeta signaling domain described herein). In one embodiment, the agent comprises a first polypeptide of PD1 or a fragment thereof (e.g., at least a portion of an extracellular domain of PD1), and a second polypeptide of an intracellular signaling domain described herein (e.g., a CD28 signaling domain described herein and/or a CD3 zeta signaling domain described herein). PD1 is an inhibitory member of the CD28 family of receptors that also includes CD28, CTLA-4, ICOS, and BTLA. PD-1 is expressed on activated B cells, T cells and myeloid cells (Agata et al. 1996 Int. Immunol 8:765-75). Two ligands for PD1, PD-L1 and PD-L2 have been shown to downregulate T cell activation upon binding to PD1 (Freeman et al. 2000 J Exp Med 192:1027-34; Latchman et al. 2001 Nat Immunol 2:261-8; Carter et al. 2002 Eur J Immunol 32:634-43). PD-L1 is abundant in human cancers (Dong et al. 2003 J Mol Med 81:281-7; Blank et al. 2005 Cancer Immunol. Immunother 54:307-314; Konishi et al. 2004 Clin Cancer Res 10:5094). Immune suppression can be reversed by inhibiting the local interaction of PD1 with PD-L1.

In one embodiment, the agent comprises the extracellular domain (ECD) of an inhibitory molecule, e.g., Programmed Death 1 (PD1) can be fused to a transmembrane domain and optionally an intracellular signaling domain such as 41BB and CD3 zeta (also referred to herein as a PD1 TFP). In one embodiment, the PD1 TFP, when used in combinations with an anti-tumor antigen TFP described herein, improves the persistence of the T cell. In one embodiment, the TFP is a PD1 TFP comprising the extracellular domain of PD 1. Alternatively, provided are TFPs containing an antibody or antibody fragment such as a scFv that specifically binds to the Programmed Death-Ligand 1 (PD-L1) or Programmed Death-Ligand 2 (PD-L2).

In another aspect, the present invention provides a population of TFP-expressing T cells, e.g., TFP-T cells. In some embodiments, the population of TFP-expressing T cells comprises a mixture of cells expressing different TFPs. For example, in one embodiment, the population of TFP-T cells can include a first cell expressing a TFP having an anti-tumor-associated antigen binding domain described herein, and a second cell expressing a TFP having a different anti-tumor-associated antigen binding domain, e.g., an anti-tumor-associated antigen binding domain described herein that differs from the anti-tumor-associated antigen binding domain in the TFP expressed by the first cell. As another example, the population of TFP-expressing cells can include a first cell expressing a TFP that includes an anti-tumor-associated antigen binding domain, e.g., as described herein, and a second cell expressing a TFP that includes an antigen binding domain to a target other than tumor-associated antigen (e.g., another tumor-associated antigen).

In another aspect, the present invention provides a population of cells wherein at least one cell in the population expresses a TFP having an anti-tumor-associated antigen domain described herein, and a second cell expressing another agent, e.g., an agent which enhances the activity of a TFP-expressing cell. For example, in one embodiment, the agent can be an agent which inhibits an inhibitory molecule. Inhibitory molecules, e.g., can, in some embodiments, decrease the ability of a TFP-expressing cell to mount an immune effector response. Examples of inhibitory molecules include PD1, PD-L1, PD-L2, CTLA4, TIM3, LAGS, VISTA, BTLA, TIGIT, LAIR1, CD160, 2B4 and TGFR beta. In one embodiment, the agent that inhibits an inhibitory molecule comprises a first polypeptide, e.g., an inhibitory molecule, associated with a second polypeptide that provides a positive signal to the cell, e.g., an intracellular signaling domain described herein.

Disclosed herein are methods for producing in vitro transcribed RNA encoding TFPs. The present invention also includes a TFP encoding RNA construct that can be directly transfected into a cell. A method for generating mRNA for use in transfection can involve in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a polyA tail, typically 50-2000 bases in length. RNA so produced can efficiently transfect different kinds of cells. In one aspect, the template includes sequences for the TFP.

In one aspect, the anti-tumor-associated antigen TFP is encoded by a messenger RNA (mRNA). In one aspect, the mRNA encoding the anti-tumor-associated antigen TFP is introduced into a T cell for production of a TFP-T cell. In one embodiment, the in vitro transcribed RNA TFP can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired template for in vitro transcription is a TFP of the present invention. In one embodiment, the DNA to be used for PCR contains an open reading frame. The DNA can be from a naturally occurring DNA sequence from the genome of an organism. In one embodiment, the nucleic acid can include some or all of the 5′ and/or 3′ untranslated regions (UTRs). The nucleic acid can include exons and introns. In one embodiment, the DNA to be used for PCR is a human nucleic acid sequence. In another embodiment, the DNA to be used for PCR is a human nucleic acid sequence including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

PCR is used to generate a template for in vitro transcription of mRNA which is used for transfection. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a nucleic acid that is normally transcribed in cells (the open reading frame), including 5′ and 3′ UTRs. The primers can also be designed to amplify a portion of a nucleic acid that encodes a particular domain of interest. In one embodiment, the primers are designed to amplify the coding region of a human cDNA, including all or portions of the 5′ and 3′ UTRs. Primers useful for PCR can be generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Any DNA polymerase useful for PCR can be used in the methods disclosed herein. The reagents and polymerase are commercially available from a number of sources.

Chemical structures with the ability to promote stability and/or translation efficiency may also be used. The RNA preferably has 5′ and 3′ UTRs. In one embodiment, the 5′ UTR is between one and 3,000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the nucleic acid of interest. Alternatively, UTR sequences that are not endogenous to the nucleic acid of interest can be added by incorporating the UTR sequences into the forward and reverse primers or by any other modifications of the template. The use of UTR sequences that are not endogenous to the nucleic acid of interest can be useful for modifying the stability and/or translation efficiency of the RNA. For example, it is known that AU-rich elements in 3′UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous nucleic acid. Alternatively, when a 5′ UTR that is not endogenous to the nucleic acid of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments, the 5′ UTR can be 5′UTR of an RNA virus whose RNA genome is stable in cells. In other embodiments, various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one preferred embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In a preferred embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenbom and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However, polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100 T tail (size can be 50-5000 Ts), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps on also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7.1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001).

### 8. Nucleic Acid Constructs Encoding a TFP

The present invention also provides nucleic acid molecules encoding one or more TFP constructs described herein. In one aspect, the nucleic acid molecule is provided as a messenger RNA transcript. In one aspect, the nucleic acid molecule is provided as a DNA construct.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The present invention also provides vectors in which a DNA of the present invention is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

In another embodiment, the vector comprising the nucleic acid encoding the desired TFP of the invention is an adenoviral vector (A5/35). In another embodiment, the expression of nucleic acids encoding TFPs can be accomplished using of transposons such as sleeping beauty, crisper, CAS9, and zinc finger nucleases (See, June et al. 2009 Nature Reviews Immunol. 9.10: 704-716, incorporated herein by reference).

The expression constructs of the present invention may also be used for nucleic acid immunization and gene therapy, using standard gene delivery protocols. Methods for gene delivery are known in the art (see, e.g., U.S. Pat. Nos. 5,399,346, 5,580,859, 5,589,466, incorporated by reference herein in their entireties). In another embodiment, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, e.g., in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

A number of virally based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

An example of a promoter that is capable of expressing a TFP transgene in a mammalian T cell is the EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving TFP expression from transgenes cloned into a lentiviral vector (see, e.g., Milone et al., Mol. Ther. 17(8): 1453-1464 (2009)). Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline-regulated promoter.

In order to assess the expression of a TFP polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art (see, e.g., Sambrook et al., 2012, Molecular Cloning: A Laboratory Manual, volumes 1-4, Cold Spring Harbor Press, NY). One method for the introduction of a polynucleotide into a host cell is calcium phosphate transfection

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like (see, e.g., U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and western blots) or by assays described herein to identify agents falling within the scope of the invention.

The present invention further provides a vector comprising a TFP encoding nucleic acid molecule. In one aspect, a TFP vector can be directly transduced into a cell, e.g., a T cell. In one aspect, the vector is a cloning or expression vector, e.g., a vector including, but not limited to, one or more plasmids (e.g., expression plasmids, cloning vectors, minicircles, minivectors, double minute chromosomes), retroviral and lentiviral vector constructs. In one aspect, the vector is capable of expressing the TFP construct in mammalian T cells. In one aspect, the mammalian T cell is a human T cell.

### 9. Sources of T Cells

Prior to expansion and genetic modification, a source of T cells is obtained from a subject. The term “subject” is intended to include living organisms in which an immune response can be elicited (e.g., mammals). Examples of subjects include humans, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain aspects of the present invention, any number of T cell lines available in the art, may be used. In certain aspects of the present invention, T cells can be obtained from a unit of blood collected from a subject using any number of techniques known to the skilled artisan, such as Ficoll™ separation. In one preferred aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and to place the cells in an appropriate buffer or media for subsequent processing steps. In one aspect of the invention, the cells are washed with phosphate buffered saline (PBS). In an alternative aspect, the wash solution lacks calcium and may lack magnesium or may lack many if not all divalent cations. Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the COBE® 2991 cell processor, the Baxter CytoMate®, or the Haemonetics® Cell Saver® 5) according to the manufacturer's instructions. After washing, the cells may be resuspended in a variety of biocompatible buffers, such as, for example, Ca-free, Mg-free PBS, PlasmaLyte® A, or other saline solution with or without buffer. Alternatively, the undesirable components of the apheresis sample may be removed and the cells directly resuspended in culture media.

In one aspect, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45RO+ T cells, can be further isolated by positive or negative selection techniques. For example, in one aspect, T cells are isolated by incubation with anti-CD³/anti-CD28 (e.g., 3×28)-conjugated beads, such as DYNABEADS™ M-450 CD3/CD28 T, for a time period sufficient for positive selection of the desired T cells. In one aspect, the time period is about 30 minutes. In a further aspect, the time period ranges from 30 minutes to 36 hours or longer and all integer values there between. In a further aspect, the time period is at least 1, 2, 3, 4, 5, or 6 hours. In yet another preferred aspect, the time period is 10 to 24 hours. In one aspect, the incubation time period is 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In certain aspects, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain aspects, it may be desirable to enrich for or positively select for regulatory T cells which typically express CD4+, CD25+, CD62Lhi, GITR+, and FoxP3+. Alternatively, in certain aspects, T regulatory cells are depleted by anti-C25 conjugated beads or other similar method of selection.

In one embodiment, a T cell population can be selected that expresses one or more of IFN-γ, TNF-alpha, IL-17A, IL-2, IL-3, IL-4, GM-CSF, IL-10, IL-13, granzyme B, and perforin, or other appropriate molecules, e.g., other cytokines. Methods for screening for cell expression can be determined, e.g., by the methods described in PCT Publication No.: WO2013/126712.

For isolation of a desired population of cells by positive or negative selection, the concentration of cells and surface (e.g., particles such as beads) can be varied. In certain aspects, it may be desirable to significantly decrease the volume in which beads and cells are mixed together (e.g., increase the concentration of cells), to ensure maximum contact of cells and beads. For example, in one aspect, a concentration of 2 billion cells/mL is used. In one aspect, a concentration of 1 billion cells/mL is used. In a further aspect, greater than 100 million cells/mL is used. In a further aspect, a concentration of cells of 10, 15, 20, 25, 30, 35, 40, 45, or 50 million cells/mL is used. In yet one aspect, a concentration of cells from 75, 80, 85, 90, 95, or 100 million cells/mL is used. In further aspects, concentrations of 125 or 150 million cells/mL can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a related aspect, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one aspect, the concentration of cells used is 5×10⁶/mL. In other aspects, the concentration used can be from about 1×10⁵/mL to 1×10⁶/mL, and any integer value in between. In other aspects, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan® and PlasmaLyte® A, the cells then are frozen to −80° C. at a rate of 1 per minute and stored in the vapor phase of a liquid nitrogen storage tank. Other methods of controlled freezing may be used as well as uncontrolled freezing immediately at −20° C. or in liquid nitrogen. In certain aspects, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one aspect, a blood sample or an apheresis is taken from a generally healthy subject. In certain aspects, a blood sample or an apheresis is taken from a generally healthy subject who is at risk of developing a disease, but who has not yet developed a disease, and the cells of interest are isolated and frozen for later use. In certain aspects, the T cells may be expanded, frozen, and used at a later time. In certain aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and tacrolimus (FK506), antibodies, or other immunoablative agents such as CAMPATH, anti-CD3 antibodies, cyclophosphamide, fludarabine, cyclosporin, rapamycin, mycophenolic acid, steroids, romidepsin (formerly FR901228), and irradiation.

In a further aspect of the present invention, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

### 10. Activation and Expansion of T Cells

T cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T cells of the invention may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol. Meth. 227(1-2):53-63, 1999).

T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8+). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

Once an anti-tumor-associated antigen TFP is constructed, various assays can be used to evaluate the activity of the molecule, such as but not limited to, the ability to expand T cells following antigen stimulation, sustain T cell expansion in the absence of re-stimulation, and anti-cancer activities in appropriate in vitro and animal models. Assays to evaluate the effects of an anti-tumor-associated antigen TFP are described in further detail below.

Western blot analysis of TFP expression in primary T cells can be used to detect the presence of monomers and dimers (see, e.g., Milone et al., Molecular Therapy 17(8): 1453-1464 (2009)). Very briefly, T cells (1:1 mixture of CD4⁺ and CD8⁺ T cells) expressing the TFPs are expanded in vitro for more than 10 days followed by lysis and SDS-PAGE under reducing conditions. TFPs are detected by western blotting using an antibody to a TCR chain. The same T cell subsets are used for SDS-PAGE analysis under non-reducing conditions to permit evaluation of covalent dimer formation.

In vitro expansion of TFP⁺ T cells following antigen stimulation can be measured by flow cytometry. For example, a mixture of CD4⁺ and CD8⁺ T cells are stimulated with alphaCD³/alphaCD28 and APCs followed by transduction with lentiviral vectors expressing GFP under the control of the promoters to be analyzed. Exemplary promoters include the CMV IE gene, EF-1alpha, ubiquitin C, or phosphoglycerokinase (PGK) promoters. GFP fluorescence is evaluated on day 6 of culture in the CD4+ and/or CD8+ T cell subsets by flow cytometry (see, e.g., Milone et al., Molecular Therapy 17(8): 1453-1464 (2009)). Alternatively, a mixture of CD4+ and CD8+ T cells are stimulated with alphaCD³/alphaCD28 coated magnetic beads on day 0, and transduced with TFP on day 1 using a bicistronic lentiviral vector expressing TFP along with eGFP using a 2A ribosomal skipping sequence.

Sustained TFP+ T cell expansion in the absence of re-stimulation can also be measured (see, e.g., Milone et al., Molecular Therapy 17(8): 1453-1464 (2009)). Briefly, mean T cell volume (fl) is measured on day 8 of culture using a Coulter Multisizer III particle counter following stimulation with alphaCD3/alphaCD28 coated magnetic beads on day 0, and transduction with the indicated TFP on day 1.

Animal models can also be used to measure a TFP-T activity. For example, xenograft model using human BCMA-specific TFP+ T cells to treat a cancer in immunodeficient mice (see, e.g., Milone et al., Molecular Therapy 17(8): 1453-1464 (2009)). Very briefly, after establishment of cancer, mice are randomized as to treatment groups. Different numbers of engineered T cells are coinfected at a 1:1 ratio into NOD/SCID/γ−/− mice bearing cancer. The number of copies of each vector in spleen DNA from mice is evaluated at various times following T cell injection. Animals are assessed for cancer at weekly intervals. Peripheral blood tumor-associated antigen+ cancer cell counts are measured in mice that are injected with alpha tumor-associated antigen-zeta TFP+ T cells or mock-transduced T cells. Survival curves for the groups are compared using the log-rank test. In addition, absolute peripheral blood CD4+ and CD8+ T cell counts 4 weeks following T cell injection in NOD/SCID/γ−/− mice can also be analyzed. Mice are injected with cancer cells and 3 weeks later are injected with T cells engineered to express TFP by a bicistronic lentiviral vector that encodes the TFP linked to eGFP. T cells are normalized to 45-50% input GFP+ T cells by mixing with mock-transduced cells prior to injection, and confirmed by flow cytometry. Animals are assessed for cancer at 1-week intervals. Survival curves for the TFP+ T cell groups are compared using the log-rank test.

Dose dependent TFP treatment response can be evaluated (see, e.g., Milone et al., Molecular Therapy 17(8): 1453-1464 (2009)). For example, peripheral blood is obtained 35-70 days after establishing cancer in mice injected on day 21 with TFP T cells, an equivalent number of mock-transduced T cells, or no T cells. Mice from each group are randomly bled for determination of peripheral blood+cancer cell counts and then killed on days 35 and 49. The remaining animals are evaluated on days 57 and 70.

Assessment of cell proliferation and cytokine production has been previously described, e.g., at Milone et al., Molecular Therapy 17(8): 1453-1464 (2009). Briefly, assessment of TFP-mediated proliferation is performed in microtiter plates by mixing washed T cells with cells expressing BCMA or CD32 and CD137 (KT32-BBL) for a final T cell:cell expressing BCMA ratio of 2:1. Cells expressing BCMA cells are irradiated with gamma-radiation prior to use. Anti-CD3 (clone OKT3) and anti-CD28 (clone 9.3) monoclonal antibodies are added to cultures with KT32-BBL cells to serve as a positive control for stimulating T cell proliferation since these signals support long-term CD8+ T cell expansion ex vivo. T cells are enumerated in cultures using CountBright™ fluorescent beads (Invitrogen) and flow cytometry as described by the manufacturer. TFP+ T cells are identified by GFP expression using T cells that are engineered with eGFP-2A linked TFP-expressing lentiviral vectors. For TFP+ T cells not expressing GFP, the TFP+ T cells are detected with biotinylated recombinant BCMA protein and a secondary avidin-PE conjugate. CD4+ and CD8+ expression on T cells are also simultaneously detected with specific monoclonal antibodies (BD Biosciences). Cytokine measurements are performed on supernatants collected 24 hours following re-stimulation using the human TH1/TH2 cytokine cytometric bead array kit (BD Biosciences) according the manufacturer's instructions. Fluorescence is assessed using a FACScalibur™ flow cytometer, and data is analyzed according to the manufacturer's instructions.

Cytotoxicity can be assessed by a standard ⁵¹Cr-release assay (see, e.g., Milone et al., Molecular Therapy 17(8): 1453-1464 (2009)). Briefly, target cells are loaded with ⁵¹Cr (as NaCrO₄, New England Nuclear) at 37° C. for 2 hours with frequent agitation, washed twice in complete RPMI and plated into microtiter plates. Effector T cells are mixed with target cells in the wells in complete RPMI at varying ratios of effector cell:target cell (E:T). Additional wells containing media only (spontaneous release, SR) or a 1% solution of triton-X 100 detergent (total release, TR) are also prepared. After 4 hours of incubation at 37° C., supernatant from each well is harvested. Released ⁵¹Cr is then measured using a gamma particle counter (Packard Instrument Co., Waltham, Mass.). Each condition is performed in at least triplicate, and the percentage of lysis is calculated using the formula: % Lysis=(ER−SR)/(TR−SR), where ER represents the average ⁵¹Cr released for each experimental condition.

Imaging technologies can be used to evaluate specific trafficking and proliferation of TFPs in tumor-bearing animal models. Such assays have been described, e.g., in Barrett et al., Human Gene Therapy 22:1575-1586 (2011). Briefly, NOD/SCID/γc−/− (NSG) mice are injected IV with cancer cells followed 7 days later with T cells 4 hour after electroporation with the TFP constructs. The T cells are stably transfected with a lentiviral construct to express firefly luciferase, and mice are imaged for bioluminescence. Alternatively, therapeutic efficacy and specificity of a single injection of TFP+ T cells in a cancer xenograft model can be measured as follows: NSG mice are injected with cancer cells transduced to stably express firefly luciferase, followed by a single tail-vein injection of T cells electroporated with BCMA TFP 7 days later. Animals are imaged at various time points post injection. For example, photon-density heat maps of firefly luciferase positive cancer in representative mice at day 5 (2 days before treatment) and day 8 (24 hours post TFP+ PBLs) can be generated.

Other assays, including those described in the Example section herein as well as those that are known in the art can also be used to evaluate the anti-BCMA TFP constructs of the invention.

### 11. Therapeutic Applications

### Tumor Antigen Associated Diseases or Disorders

Many patients treated with cancer therapeutics that are directed to one target on a tumor cell, e.g., BCMA, CD19, CD20, CD22, CD123, MUC16, MSLN, etc., become resistant over time as escape mechanisms such as alternate signaling pathways and feedback loops become activated. Dual specificity therapeutics attempt to address this by combining targets that often substitute for each other as escape routes. Therapeutic T cell populations having TCRs specific to more than one tumor-associated antigen are promising combination therapeutics. In some embodiments, the dual specificity TFP T cells are administered with an additional anti-cancer agent; in some embodiments, the anti-cancer agent is an antibody or fragment thereof, another TFP T cell, a CAR T cell, or a small molecule. Exemplary tumor-associated antigens include, but are not limited to, oncofetal antigens (e.g., those expressed in fetal tissues and in cancerous somatic cells), oncoviral antigens (e.g., those encoded by tumorigenic transforming viruses), overexpressed/accumulated antigens (e.g., those expressed by both normal and neoplastic tissue, with the level of expression highly elevated in neoplasia), cancer-testis antigens (e.g., those expressed only by cancer cells and adult reproductive tissues such as testis and placenta), lineage-restricted antigens (e.g., those expressed largely by a single cancer histotype), mutated antigens (e.g., those expressed by cancer as a result of genetic mutation or alteration in transcription), posttranslationally altered antigens (e.g., those tumor-associated alterations in glycosylation, etc.), and idiotypic antigens (e.g., those from highly polymorphic genes where a tumor cell expresses a specific clonotype, e.g., as in B cell, T cell lymphoma/leukemia resulting from clonal aberrancies). Exemplary tumor-associated antigens include, but are not limited to, antigens of alpha-actinin-4, ARTC1, alphafetoprotein (AFP), BCR-ABL fusion protein (b3a2), B-RAF, CASP-5, CASP-8, beta-catenin, Cdc27, CDK4, CDK12, CDKN2A, CLPP, COA-1, CSNK1A1, CD79, CD79B, dek-can fusion protein, EFTUD2, Elongation factor 2, ETV6-AML1 fusion protein, FLT3-ITD, FNDC3B, FN1, GAS7, GPNMB, HAUS3, HSDL1, LDLR-fucosyltransferase AS fusion protein, HLA-A2d, HLA-A11d, hsp70-2, MART2, MATN, ME1, MUM-1f, MUM-2, MUM-3, neo-PAP, Myosin class I, NFYC, OGT, OS-9, p53, pml-RARalpha fusion protein, PPP1R3B, PRDX5, PTPRK, K-ras, N-ras, RBAF600, SIRT2, SNRPD1, SYT-SSX1 or —SSX2 fusion protein, TGF-betaRII, triosephosphate isomerase, BAGE-1, D393-CD20n, Cyclin-A1, GAGE-1, GAGE-2, GAGE-8, GAGE-3, GAGE-4, GAGE-5, GAGE-6, GAGE-7, GnTVf, HERV-K-MEL, KK-LC-1, KM-HN-1, LAGE-1, LY6K, MAGE-A1, MAGE-A2, MAGE-A3, MAGE-A4, MAGE-A6, MAGE-A9, MAGE-A10, MAGE-A12 m, MAGE-C1, MAGE-C2, mucink, NA88-A, NY-ESO-1/LAGE-2, SAGE, Sp17, SSX-2, SSX-4, TAG-1, TAG-2, TRAG-3, TRP2-INT2g, XAGE-1b/GAGED2a, Gene/protein, CEA, gp100/Pme117, mammaglobin-A, Melan-A/MART-1, NY-BR-1, OA1, PAP, PSA, RAB38/NY-MEL-1, TRP-1/gp75, TRP-2, tyrosinase, adipophilin, AIM-2, ALDH1A1, BCLX (L), BING-4, CALCA, CD45, CD274, CPSF, cyclin D1, DKK1, ENAH (hMena), EpCAM, EphA3, EZH2, FGF5, glypican-3, G250/MN/CAIX, HER-2/neu, HLA-DOB, Hepsin, IDO1, IGF2B3, IL13Ralpha2, Intestinal carboxyl esterase, alpha-foetoprotein, Kallikrein 4, KIF20A, Lengsin, M-CSF, MCSP, mdm-2, Meloe, Midkine, MMP-2, MMP-7, MUC1, MUC5AC, p53, PAX5, PBF, PRAME, PSMA, RAGE-1, RGSS, RhoC, RNF43, RU2AS, secernin 1, SOX10, STEAP1, survivin, Telomerase, TPBG, VEGF, and WT1.

In one aspect, the invention provides methods for treating a disease associated with at least one tumor-associated antigen expression. In one aspect, the invention provides methods for treating a disease wherein part of the tumor is negative for the tumor associated antigen and part of the tumor is positive for the tumor associated antigen. For example, the antibody or TFP of the invention is useful for treating subjects that have undergone treatment for a disease associated with elevated expression of said tumor antigen, wherein the subject that has undergone treatment for elevated levels of the tumor associated antigen exhibits a disease associated with elevated levels of the tumor associated antigen.

In one aspect, the invention pertains to a vector comprising an anti-tumor-associated antigen antibody or TFP operably linked to promoter for expression in mammalian T cells. In one aspect, the invention provides a recombinant T cell expressing a tumor-associated antigen TFP for use in treating tumor-associated antigen-expressing tumors, wherein the recombinant T cell expressing the tumor-associated antigen TFP is termed a tumor-associated antigen TFP-T. In one aspect, the tumor-associated antigen TFP-T of the invention is capable of contacting a tumor cell with at least one tumor-associated antigen TFP of the invention expressed on its surface such that the TFP-T targets the tumor cell and growth of the tumor is inhibited.

In one aspect, the invention pertains to a method of inhibiting growth of a tumor-associated antigen-expressing tumor cell, comprising contacting the tumor cell with a tumor-associated antigen antibody or TFP T cell of the present invention such that the TFP-T is activated in response to the antigen and targets the cancer cell, wherein the growth of the tumor is inhibited.

In one aspect, the invention pertains to a method of treating cancer in a subject. The method comprises administering to the subject a tumor-associated antigen antibody, bispecific antibody, or TFP T cell of the present invention such that the cancer is treated in the subject. An example of a cancer that is treatable by the tumor-associated antigen TFP T cell of the invention is a cancer associated with expression of tumor-associated antigen. In one aspect, the cancer is a myeloma. In one aspect, the cancer is a lymphoma. In one aspect, the cancer is colon cancer.

In some embodiments, tumor-associated antigen antibodies or TFP therapy can be used in combination with one or more additional therapies. In some instances, such additional therapies comprise a chemotherapeutic agent, e.g., cyclophosphamide. In some instances, such additional therapies comprise surgical resection or radiation treatment.

In one aspect, disclosed herein is a method of cellular therapy wherein T cells are genetically modified to express a TFP and the TFP-expressing T cell is infused to a recipient in need thereof. The infused cell is able to kill tumor cells in the recipient. Unlike antibody therapies, TFP-expressing T cells are able to replicate in vivo resulting in long-term persistence that can lead to sustained tumor control. In various aspects, the T cells administered to the patient, or their progeny, persist in the patient for at least four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the T cell to the patient.

In some instances, disclosed herein is a type of cellular therapy where T cells are modified, e.g., by in vitro transcribed RNA, to transiently express a TFP and the TFP-expressing T cell is infused to a recipient in need thereof. The infused cell is able to kill tumor cells in the recipient. Thus, in various aspects, the T cells administered to the patient, is present for less than one month, e.g., three weeks, two weeks, or one week, after administration of the T cell to the patient.

Without wishing to be bound by any particular theory, the anti-tumor immunity response elicited by the TFP-expressing T cells may be an active or a passive immune response, or alternatively may be due to a direct vs indirect immune response. In one aspect, the TFP transduced T cells exhibit specific proinflammatory cytokine secretion and potent cytolytic activity in response to human cancer cells expressing the tumor-associated antigen, resist soluble tumor-associated antigen inhibition, mediate bystander killing and/or mediate regression of an established human tumor. For example, antigen-less tumor cells within a heterogeneous field of tumor-associated antigen-expressing tumor may be susceptible to indirect destruction by tumor-associated antigen-redirected T cells that has previously reacted against adjacent antigen-positive cancer cells.

In one aspect, the human TFP-modified T cells of the invention may be a type of vaccine for ex vivo immunization and/or in vivo therapy in a mammal. In one aspect, the mammal is a human.

With respect to ex vivo immunization, at least one of the following occurs in vitro prior to administering the cell into a mammal: i) expansion of the cells, ii) introducing a nucleic acid encoding a TFP to the cells or iii) cryopreservation of the cells.

Ex vivo procedures are well known in the art and are discussed more fully below. Briefly, cells are isolated from a mammal (e.g., a human) and genetically modified (i.e., transduced or transfected in vitro) with a vector expressing a TFP disclosed herein. The TFP-modified cell can be administered to a mammalian recipient to provide a therapeutic benefit. The mammalian recipient may be a human and the TFP-modified cell can be autologous with respect to the recipient. Alternatively, the cells can be allogeneic, syngeneic or xenogeneic with respect to the recipient.

The procedure for ex vivo expansion of hematopoietic stem and progenitor cells is described, e.g., in U.S. Pat. No. 5,199,942, incorporated herein by reference, can be applied to the cells of the present invention. Other suitable methods are known in the art, therefore the present invention is not limited to any particular method of ex vivo expansion of the cells. Briefly, ex vivo culture and expansion of T cells comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In addition to using a cell-based vaccine in terms of ex vivo immunization, the present invention also provides compositions and methods for in vivo immunization to elicit an immune response directed against an antigen in a patient.

Generally, the cells activated and expanded as described herein may be utilized in the treatment and prevention of diseases that arise in individuals who are immunocompromised. In particular, the TFP-modified T cells of the invention are used in the treatment of diseases, disorders and conditions associated with expression of tumor-associated antigens. In certain aspects, the cells of the invention are used in the treatment of patients at risk for developing diseases, disorders and conditions associated with expression of tumor-associated antigens. Thus, the present invention provides methods for the treatment or prevention of diseases, disorders and conditions associated with expression of tumor-associated antigens comprising administering to a subject in need thereof, a therapeutically effective amount of the TFP-modified T cells of the invention.

In one aspect, the antibodies or TFP-T cells of the inventions may be used to treat a proliferative disease such as a cancer or malignancy or is a precancerous condition. In one aspect, the cancer is a myeloma. In one aspect, the cancer is a lymphoma. In one aspect, the cancer is a colon cancer. Further, a disease associated with tumor-associated antigen expression includes, but is not limited to, e.g., atypical and/or non-classical cancers, malignancies, precancerous conditions or proliferative diseases expressing tumor-associated antigens. Non-cancer related indications associated with expression of tumor-associated antigens vary depending on the antigen, but are not limited to, e.g., infectious disease, autoimmune disease, (e.g., lupus), inflammatory disorders (allergy and asthma) and transplantation.

The antibodies or TFP-modified T cells of the present invention may be administered either alone, or as a pharmaceutical composition in combination with diluents and/or with other components such as IL-2 or IL-12 or other cytokines or cell populations.

The present invention also provides methods for inhibiting the proliferation or reducing a tumor-associated antigen-expressing cell population, the methods comprising contacting a population of cells comprising a tumor-associated antigen-expressing cell with an anti-tumor-associated antigen TFP-T cell of the invention that binds to the tumor-associated antigen-expressing cell. In a specific aspect, the present invention provides methods for inhibiting the proliferation or reducing the population of cancer cells expressing tumor-associated antigen, the methods comprising contacting the tumor-associated antigen-expressing cancer cell population with an anti-tumor-associated antigen antibody or TFP-T cell of the invention that binds to the tumor-associated antigen-expressing cell. In one aspect, the present invention provides methods for inhibiting the proliferation or reducing the population of cancer cells expressing tumor-associated antigen, the methods comprising contacting the tumor-associated antigen-expressing cancer cell population with an anti-tumor-associated antigen antibody or TFP-T cell of the invention that binds to the tumor-associated antigen-expressing cell. In certain aspects, the anti-tumor-associated antigen antibody or TFP-T cell of the invention reduces the quantity, number, amount or percentage of cells and/or cancer cells by at least 25%, at least 30%, at least 40%, at least 50%, at least 65%, at least 75%, at least 85%, at least 95%, or at least 99% in a subject with or animal model for multiple myeloma or another cancer associated with tumor-associated antigen-expressing cells relative to a negative control. In one aspect, the subject is a human.

The present invention also provides methods for preventing, treating and/or managing a disease associated with tumor-associated antigen-expressing cells (e.g., a cancer expressing tumor-associated antigen), the methods comprising administering to a subject in need an anti-tumor-associated antigen antibody or TFP-T cell of the invention that binds to the tumor-associated antigen-expressing cell. In one aspect, the subject is a human. Non-limiting examples of disorders associated with tumor-associated antigen-expressing cells include autoimmune disorders (such as lupus), inflammatory disorders (such as allergies and asthma) and cancers (such as hematological cancers or atypical cancers expressing tumor-associated antigen).

The present invention also provides methods for preventing, treating and/or managing a disease associated with tumor-associated antigen-expressing cells, the methods comprising administering to a subject in need an anti-tumor-associated antigen antibody or TFP-T cell of the invention that binds to the tumor-associated antigen-expressing cell. In one aspect, the subject is a human.

The present invention provides methods for preventing relapse of cancer associated with tumor-associated antigen-expressing cells, the methods comprising administering to a subject in need thereof an anti-tumor-associated antigen antibody or TFP-T cell of the invention that binds to the tumor-associated antigen-expressing cell. In one aspect, the methods comprise administering to the subject in need thereof an effective amount of an anti-tumor-associated antigen antibody or TFP-T cell described herein that binds to the tumor-associated antigen-expressing cell in combination with an effective amount of another therapy.

### 12. Combination Therapies

An antibody or TFP-expressing cell described herein may be used in combination with other known agents and therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In some embodiments, the “at least one additional therapeutic agent” includes a TFP-expressing cell. Also provided are T cells that express multiple TFPs, which bind to the same or different target antigens, or same or different epitopes on the same target antigen. Also provided are populations of T cells in which a first subset of T cells expresses a first TFP and a second subset of T cells expresses a second TFP.

A TFP-expressing cell described herein and the at least one additional therapeutic agent can be administered simultaneously, in the same or in separate compositions, or sequentially. For sequential administration, the TFP-expressing cell described herein can be administered first, and the additional agent can be administered second, or the order of administration can be reversed.

In further aspects, a TFP-expressing cell described herein may be used in a treatment regimen in combination with surgery, chemotherapy, radiation, immunosuppressive agents, such as cyclosporin, azathioprine, methotrexate, mycophenolate, and tacrolimus, antibodies, or other immunoablative agents such as alemtuzumab, anti-CD3 antibodies or other antibody therapies, cyclophosphamide, fludarabine, cyclosporin, tacrolimus, rapamycin, mycophenolic acid, steroids, romidepsin, cytokines, and irradiation. peptide vaccine, such as that described in Izumoto et al. 2008 J Neurosurg 108:963-971.

In one embodiment, the subject can be administered an agent which reduces or ameliorates a side effect associated with the administration of a TFP-expressing cell. Side effects associated with the administration of a TFP-expressing cell include, but are not limited to, cytokine release syndrome (CRS), and hemophagocytic lymphohistiocytosis (HLH), also termed Macrophage Activation Syndrome (MAS). Symptoms of CRS include high fevers, nausea, transient hypotension, hypoxia, and the like. Accordingly, the methods described herein can comprise administering a TFP-expressing cell described herein to a subject and further administering an agent to manage elevated levels of a soluble factor resulting from treatment with a TFP-expressing cell. In one embodiment, the soluble factor elevated in the subject is one or more of IFN-γ, TNFα, IL-2 and IL-6. Therefore, an agent administered to treat this side effect can be an agent that neutralizes one or more of these soluble factors. Such agents include, but are not limited to a steroid, an inhibitor of TNFα, and an inhibitor of IL-6. An example of a TNFα inhibitor is etanercept (marketed under the name ENBREL®). An example of an IL-6 inhibitor is tocilizumab (marketed under the name ACTEMRA®).

In one embodiment, the subject can be administered an agent which enhances the activity of a TFP-expressing cell. For example, in one embodiment, the agent can be an agent which inhibits an inhibitory molecule. Inhibitory molecules, e.g., Programmed Death 1 (PD1), can, in some embodiments, decrease the ability of a TFP-expressing cell to mount an immune effector response. Examples of inhibitory molecules include PD1, PD-L1, CTLA4, TIM3, LAG3, VISTA, BTLA, TIGIT, LAIR1, CD160, 2B4 and TGFR beta. Inhibition of an inhibitory molecule, e.g., by inhibition at the DNA, RNA or protein level, can optimize a TFP-expressing cell performance. In embodiments, an inhibitory nucleic acid, e.g., an inhibitory nucleic acid, e.g., a dsRNA, e.g., an siRNA or shRNA, can be used to inhibit expression of an inhibitory molecule in the TFP-expressing cell. In an embodiment, the inhibitor is a shRNA. In an embodiment, the inhibitory molecule is inhibited within a TFP-expressing cell. In these embodiments, a dsRNA molecule that inhibits expression of the inhibitory molecule is linked to the nucleic acid that encodes a component, e.g., all of the components, of the TFP. In one embodiment, the inhibitor of an inhibitory signal can be, e.g., an antibody or antibody fragment that binds to an inhibitory molecule. For example, the agent can be an antibody or antibody fragment that binds to PD1, PD-L1, PD-L2 or CTLA4 (e.g., ipilimumab (also referred to as MDX-010 and MDX-101, and marketed as YERVOY®; Bristol-Myers Squibb; tremelimumab (IgG2 monoclonal antibody available from Pfizer, formerly known as ticilimumab, CP-675,206)). In an embodiment, the agent is an antibody or antibody fragment that binds to T cell immunoglobulin and mucin-domain containing-3 (TIM3). In an embodiment, the agent is an antibody or antibody fragment that binds to Lymphocyte-activation gene 3 (LAG3).

In some embodiments, the agent which enhances the activity of a TFP-expressing cell can be, e.g., a fusion protein comprising a first domain and a second domain, wherein the first domain is an inhibitory molecule, or fragment thereof, and the second domain is a polypeptide that is associated with a positive signal, e.g., a polypeptide comprising an intracellular signaling domain as described herein. In some embodiments, the polypeptide that is associated with a positive signal can include a costimulatory domain of CD28, CD27, ICOS, e.g., an intracellular signaling domain of CD28, CD27 and/or ICOS, and/or a primary signaling domain, e.g., of CD3 zeta, e.g., described herein. In one embodiment, the fusion protein is expressed by the same cell that expressed the TFP. In another embodiment, the fusion protein is expressed by a cell, e.g., a T cell that does not express an anti-tumor-associated antigen TFP.

### 13. Pharmaceutical Compositions

Pharmaceutical compositions of the present invention may comprise a TFP-expressing cell, e.g., a plurality of TFP-expressing cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are in one aspect formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

In one embodiment, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD³/anti-CD28 coated beads, mouse antibodies, pooled human serum, bovine serum albumin, bovine serum, culture media components, vector packaging cell or plasmid components, a bacterium and a fungus. In one embodiment, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

When “an immunologically effective amount,” “an anti-tumor effective amount,” “a tumor-inhibiting effective amount,” or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988).

In certain aspects, it may be desired to administer activated T cells to a subject and then subsequently redraw blood (or have an apheresis performed), activate T cells therefrom according to the present invention, and reinfuse the patient with these activated and expanded T cells. This process can be carried out multiple times every few weeks. In certain aspects, T cells can be activated from blood draws of from 10 cc to 400 cc. In certain aspects, T cells are activated from blood draws of 20 cc, 30 cc, 40 cc, 50 cc, 60 cc, 70 cc, 80 cc, 90 cc, or 100 cc.

The administration of the subject compositions may be carried out in any convenient manner, including by aerosol inhalation, injection, ingestion, transfusion, implantation or transplantation. The compositions described herein may be administered to a patient trans arterially, subcutaneously, intradermally, intratumorally, intranodally, intramedullary, intramuscularly, by intravenous (i.v.) injection, or intraperitoneally. In one aspect, the T cell compositions of the present invention are administered to a patient by intradermal or subcutaneous injection. In one aspect, the T cell compositions of the present invention are administered by i.v. injection. The compositions of T cells may be injected directly into a tumor, lymph node, or site of infection.

In a particular exemplary aspect, subjects may undergo leukapheresis, wherein leukocytes are collected, enriched, or depleted ex vivo to select and/or isolate the cells of interest, e.g., T cells. These T cell isolates may be expanded by methods known in the art and treated such that one or more TFP constructs of the invention may be introduced, thereby creating a TFP-expressing T cell of the invention. Subjects in need thereof may subsequently undergo standard treatment with high dose chemotherapy followed by peripheral blood stem cell transplantation. In certain aspects, following or concurrent with the transplant, subjects receive an infusion of the expanded TFP T cells of the present invention. In an additional aspect, expanded cells are administered before or following surgery.

The dosage of the above treatments to be administered to a patient will vary with the precise nature of the condition being treated and the recipient of the treatment. The scaling of dosages for human administration can be performed according to art-accepted practices. The dose for alemtuzumab (CAMPATH®), for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

In one embodiment, the TFP is introduced into T cells, e.g., using in vitro transcription, and the subject (e.g., human) receives an initial administration of TFP T cells of the invention, and one or more subsequent administrations of the TFP T cells of the invention, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of the TFP T cells of the invention are administered to the subject (e.g., human) per week, e.g., 2, 3, or 4 administrations of the TFP T cells of the invention are administered per week. In one embodiment, the subject (e.g., human subject) receives more than one administration of the TFP T cells per week (e.g., 2, 3 or 4 administrations per week) (also referred to herein as a cycle), followed by a week of no TFP T cells administrations, and then one or more additional administration of the TFP T cells (e.g., more than one administration of the TFP T cells per week) is administered to the subject. In another embodiment, the subject (e.g., human subject) receives more than one cycle of TFP T cells, and the time between each cycle is less than 10, 9, 8, 7, 6, 5, 4, or 3 days. In one embodiment, the TFP T cells are administered every other day for 3 administrations per week. In one embodiment, the TFP T cells of the invention are administered for at least two, three, four, five, six, seven, eight or more weeks.

In one aspect, tumor-associated antigen TFP T cells are generated using lentiviral viral vectors, such as lentivirus. TFP-T cells generated that way will have stable TFP expression.

In one aspect, TFP T cells transiently express TFP vectors for 4, 5, 6, 7, 8, 9, 10, 11, 12, 13, 14, 15 days after transduction. Transient expression of TFPs can be effected by RNA TFP vector delivery. In one aspect, the TFP RNA is transduced into the T cell by electroporation.

A potential issue that can arise in patients being treated using transiently expressing TFP T cells (particularly with murine scFv bearing TFP T cells) is anaphylaxis after multiple treatments.

Without being bound by this theory, it is believed that such an anaphylactic response might be caused by a patient developing humoral anti-TFP response, i.e., anti-TFP antibodies having an anti-IgE isotype. It is thought that a patient's antibody producing cells undergo a class switch from IgG isotype (that does not cause anaphylaxis) to IgE isotype when there is a ten- to fourteen-day break in exposure to antigen.

If a patient is at high risk of generating an anti-TFP antibody response during the course of transient TFP therapy (such as those generated by RNA transductions), TFP T cell infusion breaks should not last more than ten to fourteen days.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein. Without further description, it is believed that one of ordinary skill in the art can, using the preceding description and the following illustrative examples, make and utilize the compounds of the present invention and practice the claimed methods. The following working examples specifically point out various aspects of the present invention, and are not to be construed as limiting in any way the remainder of the disclosure.

The following Examples describe engineered T cell receptors having specificity for more than one target antigen on a cancer cell; in addition are described methods of creating populations of T cells having TCRs specific for more than one antigen, either in the same cell or in a combination of cells. In one embodiment, TFP constructs are made having both binding domains (e.g., an scFv, a sdAb, etc.) in tandem on a single TCR subunit. In one embodiment, TFP constructs are made having both binding domains in a single TCR with one binding domain on each of two TCR subunits, e.g., both epsilon subunits, an epsilon and the gamma subunit, etc. In another embodiment, TFP constructs are made individually in separate lentiviral vectors, and the target T cell population is transduced with both viruses. The Examples disclose a combination of anti-MSLN TFPs and anti-MUC16 TFPs and/or a TFP having specificity to both anti-MSLN and MUC16, and/or a mixed T cell population wherein the T cells are a mix of T cells transduced with an anti-MSLN TFP and T cells transduced with an anti-MUC16 TFP. The anti-MSLN and anti-MUC16 constructs disclosed herein are exemplary only and not meant to be construed as limiting, as noted above. Constructs with a variety of combinations of anti-tumor antigen antibodies are contemplated in the methods of the invention.

### Example 1: TFP Constructs

Anti-mesothelin TFP constructs are engineered by cloning an anti-mesothelin binding domain (e.g., a sdAb, scFv, or fragment thereof) DNA fragment linked to a CD3 or TCR DNA fragment by either a DNA sequence encoding a linker having the sequence G₄S)ₙ, where n=1-4, into, e.g., a p510 vector ((System Biosciences (SBI)) at XbaI and EcoR1 sites. Other suitable vectors may be used.

The anti-mesothelin TFP constructs generated are p510_anti-mesothelin_TCRα (anti-mesothelin-linker-human full length T cell receptor α chain), p510_anti-mesothelin_TCR αC (anti-mesothelin linker-human T cell receptor α constant domain chain), p510_anti-mesothelin_TCRβ (anti-mesothelin-linker-human full length T cell receptor β chain), p510 anti-mesothelin_TCRβC (anti-mesothelin-linker-human T cell receptor β constant domain chain), p510_anti-mesothelin_TCRγ (anti-mesothelin-linker-human full length T cell receptor γ chain), p510_anti-mesothelin_TCR γC (anti-mesothelin linker-human T cell receptor γ constant domain chain), p510 anti-mesothelin_TCRδ (anti-mesothelin-linker-human full length T cell receptor δ chain), p510_anti-mesothelin_TCRδC (anti-mesothelin-linker-human T cell receptor constant domain chain), p510 anti-mesothelin_CD3γ (anti-mesothelin-linker-human CD3γ chain), p510_anti-mesothelin_CD36 (anti-mesothelin-linker-human CD3δ chain), and p510_anti-mesothelin_CD3ε (anti-mesothelin-inker-human CD3ε chain).

In some embodiments, the anti-mesothelin CAR construct, p510_antimesothelin_28ζ is generated by cloning synthesized DNA encoding anti-mesothelin, partial CD28 extracellular domain, CD28 transmembrane domain, CD28 intracellular domain and CD3 zeta into p510 vector at XbaI and EcoR1 sites. In other embodiments, the anti-mesothelin CAR construct is generated using 4-1BB zeta domain.

Anti-MUC16 TFP constructs can be engineered by cloning an anti-MUC16 binding domain (e.g., a sdAb, scFv, or fragment thereof) DNA fragment linked to a CD3 or TCR DNA fragment by a DNA sequence encoding a linker having the sequence (G4S)n, where n=1-4 into p510 vector ((System Biosciences® (SBI)) at XbaI and EcoR1 sites. Other vectors may also be used, for example, pLRPO vector.

Examples of the anti-MUC16 TFP constructs include p510_anti-MUC16_TCRα (anti-MUC16-linker-human full length T cell receptor α chain), p510_anti-MUC16_TCR αC (anti-MUC16-linker-human T cell receptor a constant domain chain), p510_antim-MUC16_TCRβ (anti-MUC16-linker-human full length T cell receptor β chain), p510_anti-MUC16_TCRβC (anti-MUC16-linker-human T cell receptor β constant domain chain), p510_anti-MUC16_TCRγ (anti-MUC16-linker-human full length T cell receptor γ chain), p510_anti-MUC16_TCR γC (anti-MUC16-linker-human T cell receptor γ constant domain chain), p510_anti-MUC16_TCRδ (anti-MUC16-linker-human full length T cell receptor δ chain), p510_anti-MUC16_TCR6C (anti-MUC16-linker-human T cell receptor β constant domain chain), p510_antimuc16_CD3γ (anti-MUC16-linker-human CD3γ chain), p510_anti-MUC16_CD3δ (anti-MUC16-linker-human CD3δ chain), and p510_anti-MUC16_CD3ε (anti-MUC16-linker-human CD3ε chain). The anti-MUC16 used herein may be a human MUC16 specific scFv, for example, 4H11.

Example of the anti-MUC16 CAR construct, p510_anti-MUC16_28ζ, can be generated by cloning synthesized DNA encoding anti-MUC16, partial CD28 extracellular domain, CD28 transmembrane domain, CD28 intracellular domain and CD3 zeta into p510 vector at XbaI and EcoR1 sites. In other embodiments, the anti-MUC16 CAR construct is generated using 4-1BB zeta domain.

Generation of TFPs from TCR Domains and Binding Domains

The MUC16 binding domains (e.g., a single domain antibody, an scFv, or fragments thereof) can be recombinantly linked to CD3-epsilon or other TCR subunits using a linker sequence, such as G₄S, (G₄S)₂ (G₄S)₃ or (G₄S)₄. If using an scFv, various linkers and scFv configurations can be used. TCR alpha and TCR beta, or TCR gamma and TCR delta, chains can be used for generation of TFPs either as full-length polypeptides or only their constant domains. Any variable sequence of TCR alpha and TCR beta/TCR gamma and TCR delta chains is suitable for making TFPs.

TFP Expression Vectors

Expression vectors are provided that include: a promoter (Cytomegalovirus (CMV) enhancer-promoter), a signal sequence to enable secretion, a polyadenylation signal and transcription terminator (Bovine Growth Hormone (BGH) gene), an element allowing episomal replication and replication in prokaryotes (e.g., SV40 origin and ColE1 or others known in the art) and elements to allow selection (ampicillin resistance gene and zeocin marker).

Preferably, the TFP-encoding nucleic acid construct is cloned into a lentiviral expression vector and expression validated based on the quantity and quality of the effector T cell response of anti-MUC16-TFP transduced T cells in response to MUC16+ target cells. Effector T cell responses include, but are not limited to, cellular expansion, proliferation, doubling, cytokine production and target cell lysis or cytolytic activity (i.e., degranulation).

The TFP.MUC16 lentiviral transfer vectors can be used to produce the genomic material packaged into the VSV-G pseudotyped lentiviral particles. Lentiviral transfer vector DNA will be mixed with the three packaging components of VSV-G, gag/pol and rev in combination with Lipofectamine® reagent to transfect them together into HEK-293 (embryonic kidney, ATCC® CRL-1573™) cells. After 24 and 48 hours, the media will be collected, filtered and concentrated by ultracentrifugation. The resulting viral preparation will be stored at −80° C. The number of transducing units can be determined by titration on Sup-T1 (T cell lymphoblastic lymphoma, ATCC® CRL-1942™) cells. Redirected TFP.MUC16 T cells will be produced by activating fresh naïve T cells with, e.g., anti-CD3 anti-CD28 beads for 24 hrs and then adding the appropriate number of transducing units to obtain the desired percentage of transduced T cells. These modified T cells will be allowed to expand until they become rested and come down in size at which point they are cryopreserved for later analysis. The cell numbers and sizes will be measured using a Coulter Multisizer™ III. Before cryopreserving, the percentage of cells transduced (expressing TFP.MUC16 on the cell surface) and the relative fluorescence intensity of that expression will be determined by flow cytometric analysis. From the histogram plots, the relative expression levels of the TFPs can be examined by comparing percentage transduced with their relative fluorescent intensity.

In some embodiments, multiple TFPs are introduced by T cell transduction with multiple viral vectors.

Evaluating Cytolytic Activity, Proliferation Capabilities and Cytokine Secretion of Humanized TFP Redirected T Cells

The functional abilities of TFP.MUC16 T cells to produce cell-surface expressed TFPs, and to kill target tumor cells, proliferate and secrete cytokines can be determined using assays known in the art.

Human peripheral blood mononuclear cells (PBMCs, e.g., blood from a normal apheresed donor whose naïve T cells can be obtained by negative selection for T cells, CD4+ and CD8+ lymphocytes) will be treated with human interleukin-2 (IL-2) then activated with anti-CD3x anti-CD28 beads, e.g., in 10% RPMI at 37° C., 5% CO₂ prior to transduction with the TFP-encoding lentiviral vectors. Flow cytometry assays can be used to confirm cell surface presence of a TFP, such as by an anti-FLAG antibody or an anti-murine variable domain antibody. Cytokine (e.g., IFN-γ) production can be measured using ELISA or other assays.

Source of TCR Subunits

Subunits of the human T Cell Receptor (TCR) complex all contain an extracellular domain, a transmembrane domain, and an intracellular domain. A human TCR complex contains the CD3-epsilon polypeptide, the CD3-gamma polypeptide, the CD3-delta polypeptide, the CD3-zeta polypeptide, the TCR alpha chain polypeptide and the TCR beta chain polypeptide. The human CD3-epsilon polypeptide canonical sequence is UniProt Accession No. P07766. The human CD3-gamma polypeptide canonical sequence is UniProt Accession No. P09693. The human CD3-delta polypeptide canonical sequence is UniProt Accession No. P043234. The human CD3-zeta polypeptide canonical sequence is UniProt Accession No. P20963. The human TCR alpha chain canonical sequence is UniProt Accession No. Q6ISU1. The human TCR beta chain C region canonical sequence is UniProt Accession No. P01850, a human TCR beta chain V region sequence is P04435.

Generation of TFPs from TCR Domains and scFvs

The mesothelin scFvs are recombinantly linked to CD3-epsilon or other TCR subunits (see 1C) using a linker sequence, such as G₄S, (G₄S)₂ (G₄S)₃ or (G₄S)₄. Various linkers and scFv configurations are utilized. TCR alpha and TCR beta chains were used for generation of TFPs either as full-length polypeptides or only their constant domains. Any variable sequence of TCR alpha and TCR beta chains is allowed for making TFPs.

TFP Expression Vectors

Expression vectors are provided that include: a promoter (Cytomegalovirus (CMV) enhancer-promoter), a signal sequence to enable secretion, a polyadenylation signal and transcription terminator (Bovine Growth Hormone (BGH) gene), an element allowing episomal replication and replication in prokaryotes (e.g., SV40 origin and ColE1 or others known in the art) and elements to allow selection (ampicillin resistance gene and zeocin marker).

Preferably, the TFP-encoding nucleic acid construct is cloned into a lentiviral expression vector and expression validated based on the quantity and quality of the effector T cell response of anti-MSLN TFP T cells in response to mesothelin+target cells. Effector T cell responses include, but are not limited to, cellular expansion, proliferation, doubling, cytokine production and target cell lysis or cytolytic activity (i.e., degranulation).

The TFP.mesothelin lentiviral transfer vectors are used to produce the genomic material packaged into the VSV-G pseudotyped lentiviral particles. Lentiviral transfer vector DNA is mixed with the three packaging components of VSV-G, gag/pol and rev in combination with Lipofectamine® reagent to transfect them together into HEK-293 (embryonic kidney, ATCC® CRL-1573™) cells. After 24 and 48 hours, the media is collected, filtered and concentrated by ultracentrifugation. The resulting viral preparation is stored at −80° C. The number of transducing units is determined by titration on Sup-T1 (T cell lymphoblastic lymphoma, ATCC® CRL-1942™) cells. Redirected TFP.mesothelin T cells are produced by activating fresh naïve T cells with, e.g., anti-CD3 anti-CD28 beads for 24 hrs and then adding the appropriate number of transducing units to obtain the desired percentage of transduced T cells. These modified T cells are allowed to expand until they become rested and come down in size at which point they are cryopreserved for later analysis. The cell numbers and sizes are measured using a Coulter Multisizer™ III. Before cryopreserving, the percentage of cells transduced (expressing TFP.mesothelin on the cell surface) and the relative fluorescence intensity of that expression are determined by flow cytometric analysis. From the histogram plots, the relative expression levels of the TFPs are examined by comparing percentage transduced with their relative fluorescent intensity.

In some embodiments multiple TFPs are introduced by T cell transduction with multiple viral vectors.

Evaluating Cytolytic Activity, Proliferation Capabilities and Cytokine Secretion of TFP Redirected T Cells

The functional abilities of anti-MSLN TFP T cells to produce cell-surface expressed TFPs, and to kill target tumor cells, proliferate and secrete cytokines are determined using assays known in the art.

Human peripheral blood mononuclear cells (PBMCs, e.g., blood from a normal apheresed donor whose naïve T cells are obtained by negative selection for T cells, CD4+ and CD8+ lymphocytes) are treated with human interleukin-2 (IL-2) then activated with anti-CD3x anti-CD28 beads, e.g., in 10% RPMI at 37° C., 5% CO₂ prior to transduction with the TFP-encoding lentiviral vectors. Flow cytometry assays are used to confirm cell surface presence of a TFP, such as by an anti-FLAG antibody or an anti-murine variable domain antibody. Cytokine (e.g., IFN-γ) production is measured using ELISA or other assays.

### Example 2: Antibody Sequences

Generation of Antibody Sequences

The human mesothelin polypeptide canonical sequence is UniProt Accession No. Q13421 (or Q13421-1). Provided are antibody polypeptides that are capable of specifically binding to the human mesothelin polypeptide, and fragments or domains thereof. Anti-mesothelin antibodies can be generated using diverse technologies (see, e.g., (Nicholson et al, 1997). Where anti-mesothelin antibodies made in mouse, camel, or other species are used as a starting material, humanization is performed. For example, humanization of murine anti-mesothelin antibodies is desired for the clinical setting, where the mouse-specific residues may induce a human-anti-mouse antigen (HAMA) response in subjects who receive T cell receptor (TCR) fusion protein (TFP) treatment, i.e., treatment with T cells transduced with the anti-MSLN/anti-MUC16 TFP construct. Humanization is accomplished by grafting CDR regions from a non-human anti-mesothelin antibody onto appropriate human germline acceptor frameworks, optionally including other modifications to CDR and/or framework regions. As provided herein, antibody and antibody fragment residue numbering follows Kabat (Kabat E. A. et al, 1991; Chothia et al, 1987).

Generation of scFvs

Human or humanized anti-mesothelin IgGs are used to generate scFv sequences for TFP constructs. DNA sequences coding for human or humanized V_{L} and V_{H} domains are obtained, and the codons for the constructs are, optionally, optimized for expression in cells from Homo sapiens. The order in which the V_{L} and V_{H} domains appear in the scFv is varied (i.e., V_{L}-V_{H}, or V_{H}-V_{L} orientation), and three copies of the “G4S” or “G₄S” subunit (G₄S)₃ connect the variable domains to create the scFv domain. Anti-mesothelin or anti-MUC16 scFv plasmid constructs can have optional Flag, His or other affinity tags, and are electroporated into HEK293 or other suitable human or mammalian cell lines and purified. Validation assays include binding analysis by FACS, kinetic analysis using Proteon®, and staining of mesothelin-expressing cells.

Exemplary anti-mesothelin V_{L} and V_{H} domains, CDRs, and the nucleotide sequences encoding them, can be those described in U.S. Pat. Nos. 9,272,002; 8,206,710; 9,023,351; 7,081,518; 8,911,732; 9,115,197 and 9,416,190; and U.S. Patent Publication No. 20090047211. Other exemplary anti-mesothelin V_{L} and Vu domains, CDRs, and the nucleotide sequences encoding them, respectively, can be those of the following monoclonal antibodies: rat anti-mesothelin antibody 420411, rat anti-mesothelin antibody 420404, mouse anti-mesothelin antibody MN-1, mouse anti-mesothelin antibody MB-G10, mouse anti-mesothelin antibody ABIN233753, rabbit anti-mesothelin antibody FQS3796(3), rabbit anti-mesothelin antibody TQ85, mouse anti-mesothelin antibody TA307799, rat anti-mesothelin antibody 295D, rat anti-mesothelin antibody B35, mouse anti-mesothelin antibody 5G157, mouse anti-mesothelin antibody 129588, rabbit anti-mesothelin antibody 11C187, mouse anti-mesothelin antibody 5B2, rabbit anti-mesothelin antibody SP74, rabbit anti-mesothelin antibody D4X7M, mouse anti-mesothelin antibody C-2, mouse anti-mesothelin antibody C-3, mouse anti-mesothelin antibody G-1, mouse anti-mesothelin antibody G-4, mouse anti-mesothelin antibody K1, mouse anti-mesothelin antibody B-3, mouse anti-mesothelin antibody 200-301-A87, mouse anti-mesothelin antibody 200-301-A88, rabbit anti-mesothelin antibody EPR2685(2), rabbit anti-mesothelin antibody EPR4509, or rabbit anti-mesothelin antibody PPI-2e(IHC).

In some embodiments, single-domain (V_{HH}) binders are used such as those set forth in SEQ ID NOS 52-54 (SD1, SD4, and SD6, respectively).

Human or humanized anti-MUC16 IgGs can be used to generate scFv sequences for TFP constructs. DNA sequences coding for human or humanized V_{L} and V_{H} domains are obtained, and the codons for the constructs are, optionally, optimized for expression in cells from Homo sapiens. The order in which the V_{L} and V_{H} domains appear in the scFv is varied (i.e., V_{L}-V_{H}, or V_{H}-V_{L} orientation), and three copies of the “G4S” or “G₄S” subunit (G₄S)₃ connect the variable domains to create the scFv domain. Anti-MUC16 scFv plasmid constructs can have optional Flag, His or other affinity tags, and are electroporated into HEK293 or other suitable human or mammalian cell lines and purified. Validation assays include binding analysis by FACS, kinetic analysis using Proteon, and staining of MUC16-expressing cells.

Examples of anti-MUC16 binding domains, including V_{L} domain, V_{H} domain, and CDRs, that can be used with the compositions and methods described herein can be in some publications and/or commercial sources. For example, certain anti-MUC16 antibodies, including 3A5 and 11D10, have been disclosed in WO 2007/001851, the contents of which are incorporated by reference. The 3A5 monoclonal antibody binds multiple sites of the MUC16 polypeptide with 433 pM affinity by OVCAR-3 Scatchard analysis. Other examples of anti-MUC16 VL and VH domains, CDRs and the nucleotide sequences encoding them, respectively, can be those of the following monoclonal antibodies: GTX10029, GTX21107, MA5-124525, MA5-11579, 25450002, ABIN1584127, ABIN93655, 112889, 120204, LS-C356195, LS-B6756, TA801241, TA801279, V3494, V3648, 666902, 666904, HPA065600, AMAb91056.

The human MUC16 polypeptide canonical sequence corresponds to UniProt Accession No. Q8WXI7. Provided are antibody polypeptides that are capable of specifically binding to the human MUC16 polypeptide, and fragments or domains thereof. Anti-MUC16 antibodies can be generated using diverse technologies (see, e.g., (Nicholson et al, 1997). Where murine anti-MUC16 antibodies are used as a starting material, humanization of murine anti-MUC16 antibodies is desired for the clinical setting, where the mouse-specific residues may induce a human-anti-mouse antigen (HAMA) response in subjects who receive T cell receptor (TCR) fusion protein (TFP) treatment, i.e., treatment with T cells transduced with the TFP.MUC16 construct. Humanization is accomplished by grafting CDR regions from murine anti-MUC16 antibody onto appropriate human germline acceptor frameworks, optionally including other modifications to CDR and/or framework regions. As provided herein, antibody and antibody fragment residue numbering follows Kabat (Kabat E. A. et al, 1991; Chothia et al, 1987).

Single Domain Binders

Camelid or other single domain antibodies can also be used to generate anti-MUC16 TFP constructs. The V_{HH} domain can be used to be fused with various TCR subunits. In some embodiments, single-domain (e.g., V_{HH}) binders are used such as those set forth in Table 2 (SEQ ID NO:14, SEQ ID NO:19, SEQ ID NO:24, SEQ ID NO:29, SEQ ID NO:34, SEQ 1D NO:39, SEQ ID NO:43, and SEQ ID NO:47). The preparation of anti-hMUC16 camelid antibodies is further described in Example 3.

Generation of TFPs from TCR Domains and scFvs

The MUC16 scFvs can be recombinantly linked to CD3-epsilon or other TCR subunits using a linker sequence, such as G₄S, (G₄S)₂ (G₄S)₃ or (G₄S)₄. Various linkers and scFv configurations can be utilized. TCR alpha and TCR beta chains can be used for generation of TFPs either as full-length polypeptides or only their constant domains. Any variable sequence of TCR alpha and TCR beta chains is allowed for making TFPs.

TFP Expression Vectors

Expression vectors are provided that include: a promoter (Cytomegalovirus (CMV) enhancer-promoter), a signal sequence to enable secretion, a polyadenylation signal and transcription terminator (Bovine Growth Hormone (BGH) gene), an element allowing episomal replication and replication in prokaryotes (e.g., SV40 origin and ColE1 or others known in the art) and elements to allow selection (ampicillin resistance gene and zeocin marker).

Preferably, the TFP-encoding nucleic acid construct is cloned into a lentiviral expression vector and expression validated based on the quantity and quality of the effector T cell response of TFP.MUC16-transduced T cells (“MUC16.TFP” or “MUC16.TFP T cells” or “TFP.MUC16” or “TFP.MUC16 T cells”) in response to MUC16+ target cells. Effector T cell responses include, but are not limited to, cellular expansion, proliferation, doubling, cytokine production and target cell lysis or cytolytic activity (i.e., degranulation).

The TFP.MUC16 lentiviral transfer vectors can be used to produce the genomic material packaged into the VSV-G pseudotyped lentiviral particles. Lentiviral transfer vector DNA will be mixed with the three packaging components of VSV-G, gag/pol and rev in combination with Lipofectamine® reagent to transfect them together into HEK-293 (embryonic kidney, ATCC® CRL-1573™) cells. After 24 and 48 hours, the media will be collected, filtered and concentrated by ultracentrifugation. The resulting viral preparation will be stored at −80° C. The number of transducing units can be determined by titration on Sup-T1 (T cell lymphoblastic lymphoma, ATCC® CRL-1942™) cells. Redirected TFP.MUC16 T cells will be produced by activating fresh naïve T cells with, e.g., anti-CD3 anti-CD28 beads for 24 hrs and then adding the appropriate number of transducing units to obtain the desired percentage of transduced T cells. These modified T cells will be allowed to expand until they become rested and come down in size at which point they are cryopreserved for later analysis. The cell numbers and sizes will be measured using a Coulter Multisizer™ III. Before cryopreserving, the percentage of cells transduced (expressing TFP.MUC16 on the cell surface) and the relative fluorescence intensity of that expression will be determined by flow cytometric analysis. From the histogram plots, the relative expression levels of the TFPs can be examined by comparing percentage transduced with their relative fluorescent intensity.

In some embodiments, multiple TFPs are introduced by T cell transduction with multiple viral vectors.

Evaluating Cytolytic Activity, Proliferation Capabilities and Cytokine Secretion of Humanized TFP Redirected T Cells

The functional abilities of TFP.MUC16 T cells to produce cell-surface expressed TFPs, and to kill target tumor cells, proliferate and secrete cytokines can be determined using assays known in the art.

Human peripheral blood mononuclear cells (PBMCs, e.g., blood from a normal apheresed donor whose naïve T cells can be obtained by negative selection for T cells, CD4+ and CD8+ lymphocytes) will be treated with human interleukin-2 (IL-2) then activated with anti-CD3x anti-CD28 beads, e.g., in 10% RPMI at 37° C., 5% CO₂ prior to transduction with the TFP-encoding lentiviral vectors. Flow cytometry assays can be used to confirm cell surface presence of a TFP, such as by an anti-FLAG antibody or an anti-murine variable domain antibody. Cytokine (e.g., IFN-γ) production can be measured using ELISA or other assays.

Source of TCR Subunits

Subunits of the human T Cell Receptor (TCR) complex all contain an extracellular domain, a transmembrane domain, and an intracellular domain. A human TCR complex contains the CD3-epsilon polypeptide, the CD3-gamma polypeptide, the CD3-delta polypeptide, the CD3-zeta polypeptide, the TCR alpha chain polypeptide and the TCR beta chain polypeptide. The human CD3-epsilon polypeptide canonical sequence is UniProt Accession No. P07766. The human CD3-gamma polypeptide canonical sequence is UniProt Accession No. P09693. The human CD3-delta polypeptide canonical sequence is UniProt Accession No. P043234. The human CD3-zeta polypeptide canonical sequence is UniProt Accession No. P20963. The human TCR alpha chain canonical sequence is UniProt Accession No. Q6ISU1. The human TCR beta chain C region canonical sequence is UniProt Accession No. P01850, a human TCR beta chain V region sequence is P04435.

Generation of TFPs from TCR Domains and scFvs

The mesothelin scFvs are recombinantly linked to CD3-epsilon or other TCR subunits (see 1C) using a linker sequence, such as G₄S, (G₄S)₂ (G₄S)₃ or (G₄S)₄. Various linkers and scFv configurations are utilized. TCR alpha and TCR beta chains were used for generation of TFPs either as full-length polypeptides or only their constant domains. Any variable sequence of TCR alpha and TCR beta chains is allowed for making TFPs.

TFP Expression Vectors

Expression vectors are provided that include: a promoter (Cytomegalovirus (CMV) enhancer-promoter), a signal sequence to enable secretion, a polyadenylation signal and transcription terminator (Bovine Growth Hormone (BGH) gene), an element allowing episomal replication and replication in prokaryotes (e.g., SV40 origin and ColE1 or others known in the art) and elements to allow selection (ampicillin resistance gene and zeocin marker).

Preferably, the TFP-encoding nucleic acid construct is cloned into a lentiviral expression vector and expression validated based on the quantity and quality of the effector T cell response of anti-MSLN TFP T cells in response to mesothelin+target cells. Effector T cell responses include, but are not limited to, cellular expansion, proliferation, doubling, cytokine production and target cell lysis or cytolytic activity (i.e., degranulation).

The TFP.mesothelin lentiviral transfer vectors are used to produce the genomic material packaged into the VSV-G pseudotyped lentiviral particles. Lentiviral transfer vector DNA is mixed with the three packaging components of VSV-G, gag/pol and rev in combination with Lipofectamine® reagent to transfect them together into HEK-293 (embryonic kidney, ATCC® CRL-1573™) cells. After 24 and 48 hours, the media is collected, filtered and concentrated by ultracentrifugation. The resulting viral preparation is stored at −80° C. The number of transducing units is determined by titration on Sup-T1 (T cell lymphoblastic lymphoma, ATCC® CRL-1942™) cells. Redirected TFP.mesothelin T cells are produced by activating fresh naïve T cells with, e.g., anti-CD3 anti-CD28 beads for 24 hrs and then adding the appropriate number of transducing units to obtain the desired percentage of transduced T cells. These modified T cells are allowed to expand until they become rested and come down in size at which point they are cryopreserved for later analysis. The cell numbers and sizes are measured using a Coulter Multisizer™ III. Before cryopreserving, the percentage of cells transduced (expressing TFP.mesothelin on the cell surface) and the relative fluorescence intensity of that expression are determined by flow cytometric analysis. From the histogram plots, the relative expression levels of the TFPs are examined by comparing percentage transduced with their relative fluorescent intensity.

In some embodiments multiple TFPs are introduced by T cell transduction with multiple viral vectors.

Evaluating Cytolytic Activity, Proliferation Capabilities and Cytokine Secretion of TFP Redirected T Cells

The functional abilities of anti-MSLN TFP T cells to produce cell-surface expressed TFPs, and to kill target tumor cells, proliferate and secrete cytokines are determined using assays known in the art.

Human peripheral blood mononuclear cells (PBMCs, e.g., blood from a normal apheresed donor whose naïve T cells are obtained by negative selection for T cells, CD4+ and CD8+ lymphocytes) are treated with human interleukin-2 (IL-2) then activated with anti-CD3x anti-CD28 beads, e.g., in 10% RPMI at 37° C., 5% CO₂ prior to transduction with the TFP-encoding lentiviral vectors. Flow cytometry assays are used to confirm cell surface presence of a TFP, such as by an anti-FLAG antibody or an anti-murine variable domain antibody. Cytokine (e.g., IFN-γ) production is measured using ELISA or other assays.

### Example 3: Demonstration of Multiplexed TFP Polypeptides, and Use of Multiplexed Humanized TFP Redirected T Cells

The TFP polypeptides provided herein are capable of functionally associating with endogenous TCR subunit polypeptides to form functional TCR complexes. Here, multiple TFPs in lentiviral vectors are used to transduce T cells in order to create a functional, multiplexed recombinant TCR complex. For example, provided is a T cell containing i) a first TFP having an extracellular domain, a transmembrane domain, and an intracellular domain from, e.g., the CD3-epsilon polypeptide and a mesothelin-specific scFv antibody fragment, and ii) a second TFP having an extracellular domain, a transmembrane domain, and an intracellular domain from the CD3-gamma polypeptide and a mesothelin-specific antibody fragment. The first TFP and second TFP are capable of interacting with each other and with endogenous TCR subunit polypeptides, thereby forming a functional TCR complex.

The use of these multiplexed humanized anti-MSLN, anti-MUC16 TFP T cells can be demonstrated in solid tumors.

### Example 4: Preparation of T Cells Transduced with TFPs

Lentiviral Production

Lentivirus encoding the appropriate constructs are prepared as follows. 5×10⁶ HEK-293FT cells are seeded into a 100 mm dish and allowed to reach 70-90% confluency overnight. 2.5 μg of the indicated DNA plasmids and 20 μL Lentivirus Packaging Mix (ALSTEM, cat #VP100) are diluted in 0.5 mL DMEM or Opti-MEM® I Medium without serum and mixed gently. In a separate tube, 30 μL of NanoFect® transfection reagent (ALSTEM, cat #NF100) is diluted in 0.5 mL DMEM or Opti-MEM® I Medium without serum and mixed gently. The NanoFect/DMEM and DNA/DMEM solutions are then mixed together and vortexed for 10-15 seconds prior to incubation of the DMEM-plasmid-NanoFect mixture at room temperature for 15 minutes. The complete transfection complex from the previous step is added dropwise to the plate of cells and rocked to disperse the transfection complex evenly in the plate. The plate is then incubated overnight at 37° C. in a humidified 5% CO₂ incubator. The following day, the supernatant is replaced with 10 mL fresh media and supplemented with 20 μL of ViralBoost (500×, ALSTEM, cat #VB100). The plates are then incubated at 37° C. for an additional 24 hours. The lentivirus containing supernatant is then collected into a 50 mL sterile, capped conical centrifuge tube and put on ice. After centrifugation at 3000 rpm for 15 minutes at 4° C., the cleared supernatant is filtered with a low-protein binding 0.45 μm sterile filter and virus is subsequently isolated by ultracentrifugation at 25,000 rpm (Beckmann, L8-70M) for 1.5 hours, at 4° C. The pellet is removed and re-suspended in DMEM media and lentivirus concentrations/titers are established by quantitative RT-PCR, using the Lenti-X™ qRT-PCR Titration kit (Clontech®; catalog number 631235). Any residual plasmid DNA is removed by treatment with DNaseI. The virus stock preparation is either used for infection immediately or aliquoted and stored at −80° C. for future use.

PBMC Isolation

Peripheral blood mononuclear cells (PBMCs) are prepared from either whole blood or buffy coat. Whole blood is collected in 10 mL Heparin vacutainers and either processed immediately or stored overnight at 4° C. Approximately 10 mL of whole anti-coagulated blood is mixed with sterile phosphate buffered saline (PBS) buffer for a total volume of 20 mL in a 50 mL conical centrifuge tube (PBS, pH 7.4, without Ca²⁺/Mg²⁺). 20 mL of this blood/PBS mixture is then gently overlaid onto the surface of 15 mL of Ficoll-Paque® PLUS (GE Healthcare®, 17-1440-03) prior to centrifugation at 400 g for 30-40 min at room temperature with no brake application.

Buffy coat is purchased from Research Blood Components (Boston, Mass.). LeucoSep® tubes (Greiner bio-one) are prepared by adding 15 mL Ficoll-Paque® (GE Health Care) and centrifuged at 1000 g for 1 minute. Buffy coat is diluted 1:3 in PBS (pH 7.4, without Ca²⁺ or Mg²⁺). The diluted buffy coat is transferred to Leucosep tube and centrifuged at 1000 g for 15 minutes with no brake application. The layer of cells containing PBMCs, seen at the diluted plasma/Ficoll interface, is removed carefully to minimize contamination by Ficoll. Residual Ficoll, platelets, and plasma proteins are then removed by washing the PBMCs three times with 40 mL of PBS by centrifugation at 200 g for 10 minutes at room temperature. The cells are then counted with a hemocytometer. The washed PBMC are washed once with CAR-T media (AIM V-AlbuMAX® (BSA) (Life Technologies), with 5% AB serum and 1.25 μg/mL amphotericin B (Gemini Bio-products, Woodland, Calif.), 100 U/mL penicillin, and 100 μg/mL streptomycin). Alternatively, the washed PBMC's are transferred to insulated vials and frozen at −80° C. for 24 hours before storing in liquid nitrogen for later use.

T Cell Activation

PBMCs prepared from either whole blood or buffy coat are stimulated with anti-human CD28 and CD3 antibody-conjugated magnetic beads for 24 hours prior to viral transduction. Freshly isolated PBMC are washed once in CAR-T media (AIM V-AlbuMAX (BSA) (Life Technologies), with 5% AB serum and 1.25 μg/mL amphotericin B (Gemini Bio-products), 100 U/mL penicillin, and 100 μg/mL streptomycin) without huIL-2, before being re-suspended at a final concentration of 1×10⁶ cells/mL in CAR-T medium with 300 IU/mL human IL-2 (from a 1000×stock; Invitrogen). If the PBMCs had previously been frozen they are thawed and re-suspended at 1×10⁷ cells/mL in 9 mL of pre-warmed (37° C.) cDMEM media (Life Technologies), in the presence of 10% FBS, 100 U/mL penicillin, and 100 μg/mL streptomycin, at a concentration of 1×10⁶ cells/mL prior to washing once in CAR-T medium, re-suspension at 1×10⁶ cells/mL in CAR-T medium, and addition of IL-2 as described above.

Prior to activation, anti-human CD28 and CD3 antibody-conjugated magnetic beads (available from, e.g., Invitrogen, Life Technologies) are washed three times with 1 mL of sterile 1×PBS (pH 7.4), using a magnetic rack to isolate beads from the solution, before re-suspension in CAR-T medium, with 300 IU/mL human IL-2, to a final concentration of 4×10⁷ beads/mL. PBMC and beads are then mixed at a 1:1 bead-to-cell ratio, by transferring 25 μL (1×10⁶ beads) of beads to 1 mL of PBMC. The desired number of aliquots are then dispensed to single wells of a 12-well low-attachment or non-treated cell culture plate, and incubated at 37° C., with 5% CO₂, for 24 hours before viral transduction.

T Cell Transduction/Transfection and Expansion

Following activation of PBMC, cells are incubated for 48 hours at 37° C., 5% CO₂. Lentivirus is thawed on ice and 5×10⁶ lentivirus, along with 2 μL of TransPlus™ (Alstem) per mL of media (a final dilution of 1:500) is added to each well of 1×10⁶ cells. Cells are incubated for an additional 24 hours before repeating addition of virus. Alternatively, lentivirus is thawed on ice and the respective virus is added at 5 or 50 MOI in presence of 5 μg/mL polybrene (Sigma). Cells are spinoculated at 100 g for 100 minutes at room temperature. Cells are then grown in the continued presence of 300 IU/mL of human IL-2 for a period of 6-14 days (total incubation time is dependent on the final number of CAR-T cells required). Cell concentrations are analyzed every 2-3 days, with media being added at that time to maintain the cell suspension at 1×10⁶ cells/mL.

In some instances, activated PBMCs are electroporated with in vitro transcribed (IVT) mRNA. In one embodiment, human PBMCs are stimulated with Dynabeads® (Thermo Fisher Scientific®) at 1-to-1 ratio for 3 days in the presence of 300 IU/ml recombinant human IL-2 (R&D Systems) (other stimulatory reagents such as TransAct® T Cell Reagent from Milyeni Biotec may be used). The beads are removed before electroporation. The cells are washed and re-suspended in OPTI-MEM medium (Thermo Fisher Scientific) at the concentration of 2.5×10⁷ cells/mL. 200 μL of the cell suspension (5×10⁶ cells) are transferred to the 2 mm gap Electroporation Cuvettes Plus™ (Harvard Apparatus® BTX) and prechilled on ice. 10 μs of IVT TFP mRNA is added to the cell suspension. The mRNA/cell mixture is then electroporated at 200 V for 20 milliseconds using ECM® 830 Electro Square Wave Porator (Harvard Apparatus BTX). Immediately after the electroporation, the cells are transferred to fresh cell culture medium (AIM V AlbuMAX® (BSA) serum free medium+5% human AB serum+300 IU/ml IL-2) and incubated at 37° C.

Verification of TFP Expression by Cell Staining

Following lentiviral transduction or mRNA electroporation, expression of anti-mesothelin or MUC16 TFPs is confirmed by flow cytometry, using an anti-mouse Fab antibody to detect the murine anti-mesothelin or MUC16. T cells are washed three times in 3 mL staining buffer (PBS, 4% BSA) and re-suspended in PBS at 1×10⁶ cells per well. For dead cell exclusion, cells are incubated with LIVE/DEAD® Fixable Aqua Dead Cell Stain (Invitrogen) for 30 minutes on ice. Cells are washed twice with PBS and re-suspended in 50 μL staining buffer. To block Fc receptors, 1 μL of 1:100 diluted normal goat lgG (BD Bioscience) is added to each tube and incubated in ice for 10 minutes. 1.0 mL FACS buffer is added to each tube, mixed well, and cells are pelleted by centrifugation at 300 g for 5 min. Surface expression of scFv TFPs is detected by Zenon® R-Phycoerythrin-labeled human MSLN IgG1 Fc or human IgG1 isotype control. 1 μg antibodies are added to the respective samples and incubated for 30 minutes on ice. Cells are then washed twice and stained for surface markers using Anti-CD3 APC (clone, UCHT1), anti-CD4-Pacific blue (Clone RPA-T4), nti-CD8 APCCy7(Clone SK1), from BD® bioscience. Flow cytometry is performed using LSRFortessa™ X20 (BD Biosciences) and data is acquired using FACSDiva® software and is analyzed with FlowJo® (Treestar, Inc. Ashland, Oreg.).

### Example 5: Cytotoxicity Assay by Flow Cytometry

Target cells that are either positive or negative for mesothelin or MUC16 are labelled with the fluorescent dye, carboxyfluorescein diacetate succinimidyl ester (CFSE). These target cells are mixed with effector T cells that are either un-transduced, transduced with control CAR-T constructs, or transduced with TFPs. After the indicated incubation period, the percentage of dead to live CFSE-labeled target cells and negative control target cells is determined for each effector/target cell culture by flow cytometry. The percent survival of target cells in each T cell-positive target cell culture is calculated relative to wells containing target cells alone.

The cytotoxic activity of effector T cells is measured by comparing the number of surviving target cells in target cells without or with effector T cells, following co-incubation of effector and target cells, using flow cytometry. In experiments with mesothelin.MUC16 TFPs or CAR-T cells, the target cells are mesothelin or MUC16-positive cells, while cells used as a negative control are mesothelin or MUC16-negative cells.

Target cells are washed once and re-suspended in PBS at 1×10⁶ cells/mL. The fluorescent dye carboxyfluorescein diacetate succinimidyl ester (CFSE) (Thermo Fisher Scientific®) is added to the cell suspension at a concentration of 0.03 μM and the cells are incubated for 20 minutes at room temperature. The labeling reaction is stopped by adding to the cell suspension complete cell culture medium (RPMI®-1640+10% HI-FBS) at the volume 5 times of the reaction volume, and the cells are incubated for an additional two minutes at room temperature. The cells are pelleted by centrifugation and re-suspended in cytotoxicity medium (phenol red-free RPMI-1640 (Invitrogen®) plus 5% AB serum (Gemini Bio-products) at 2×10⁵ cells/mL. Fifty microliters of CFSE labelled-target cell suspension (equivalent to 10,000 cells) are added to each well of the 96-well U-bottom plate (Corning® Life Sciences).

Effector T cells transduced with TFP constructs, together with non-transduced T cells as negative controls, are washed and suspended at 2×10⁶ cells/mL, or 1×10⁶ cells/mL, in cytotoxicity medium. 50 μL of effector T cell suspensions (equivalent to 100,000 or 50,000 cells) are added to the plated target cells to reach the effector-to-target ratio of 10-to-1 or 5-to-1, respectively, in a total volume of 100 μL. The cultures are then mixed, spun down, and incubated for four hours at 37° C. and 5% CO₂. Immediately following this incubation, 7AAD (7-aminoactinomycin D) (BioLegend®) is added to the cultured cells as recommended by the manufacturer, and flow cytometry is performed with a BD LSRFortessa® X-20 (BD® Biosciences). Analysis of flow cytometric data is performed using FlowJo® software (TreeStar, Inc.).

The percentage of survival for target cells is calculated by dividing the number of live target cells (CFSE+7-AAD-) in a sample with effector T cells and target cells, by the number of live (CFSE+7-AAD-) cells in the sample with target cells alone. The cytotoxicity for effector cells is calculated as the percentage of killing for target cells=100%−percentage of survival for the cells.

T cells transduced with an anti-MSLN.MUC16 28ζ CAR construct or an anti-MSLN anti-MUC16 BBζ CAR construct may demonstrate cytotoxicity against mesothelin- or MUC16-expressing cells when compared to T cells that are either non-transduced or are transduced with a non-mesothelin or MUC16-specific CAR control. However, T cells transduced with anti-mesothelin-CD3ε and anti-MUC16-CD3ε may induce more efficient cytotoxicity against the targets than the anti-mesothelin CAR control. Anti-mesothelin-CD3γ and anti-MUC16-CD3γ TFPs may also mediate robust cytotoxicity that is greater than that observed with anti-mesothelin and anti-MUC16-CAR at effector:target ratios between 5 and 10:1. Similar results may be obtained with TFPs constructed with an alternative hinge region. Once again, cytotoxicity against mesothelin or MUC16-expressing target cells may be greater with anti-mesothelin-CD3ε and anti-MUC16-CD3ε or anti-mesothelin-CD3γ and anti-MUC16-CD3γ TFP-transduced T cells than with anti-mesothelin- and anti-MUC16-CAR-transduced T cells.

T cells electroporated with mRNA encoding TFPs specific for mesothelin and MUC16 may also demonstrate robust cytotoxicity against mesothelin-expressing cells. While no significant killing of the mesothelin-negative cells may be seen with either control or anti-mesothelin and anti-MUC16 TFP constructs, mesothelin- or MUC16-specific killing of mesothelin or MUC16-expressing cells may be observed by T cells transduced with either anti-mesothelin and anti-MUC16-CD3ε, or anti-mesothelin- and anti-MUC16 CD3γ TFPs.

### Example 6: Determining Cytotoxicity by Real Time Cytotoxicity Assay

TFPs may also demonstrate superior cytotoxicity over CARs in the real-time cytotoxicity assay (RTCA) format. The RTCA assay measures the electrical impedance of an adherent target cell monolayer, in each well of a specialized 96-well plate, in real time and presents the final readout as a value called the cell index. Changes in cell index indicate disruption of the target cell monolayer as a result of killing of target cells by co-incubated T cell effectors. Thus, the cytotoxicity of the effector T cells can be evaluated as the change in cell index of wells with both target cells and effector T cells compared to that of wells with target cells alone.

Adherent target cells are cultured in DMEM, 10% FBS, 1% Antibiotic-Antimycotic (Life Technologies). To prepare the RTCA, 50 μL of, e.g., DMEM medium is added into the appropriate wells of an E-plate (ACEA Biosciences®, Inc, Catalog #: JL-10-156010-1A). The plate is then placed into a RTCA MP instrument (ACEA Biosciences, Inc.) and the appropriate plate layout and assay schedule entered into the RTCA 2.0 software as described in the manufacturer's manual. Baseline measurement is performed every 15 minutes for 100 measurements. 1×10⁴ target cells in a 100 μL volume are then added to each assay well and the cells are allowed to settle for 15 minutes. The plate is returned to the reader and readings are resumed.

The next day, effector T cells are washed and re-suspended in cytotoxicity media (Phenol red-free RPMI1640 (Invitrogen®) plus 5% AB serum (Gemini Bio-products; 100-318)). The plate is then removed from the instrument and the effector T cells, suspended in cytotoxicity medium (Phenol red-free RPMI®-1640+5% AB serum), are added to each well at 100,000 cells or 50,000 cells to reach the effector-to-target ratio of 10-to-1 or 5-to-1, respectively. The plate is then placed back to the instrument. The measurement is carried out for every 2 minutes for 100 measurements, and then every 15 minutes for 1,000 measurements.

In the RTCA assay, killing of TFP-transduced cells may be observed by T cells transduced with anti-mesothelin-28ζ and anti-MUC16-28ζ CAR-transduced T cells, or anti-mesothelin-BBζ and anti-MUC16 BBζ CAR-transduced constructs, as demonstrated by a time-dependent decrease in the cell index following addition of the effector cells relative to cells alone or cells co-incubated with T cells transduced with a control CAR construct. However, target cell killing by TFP-expressing T cells may be deeper and more rapid than that observed with the CAR. For example, within 4 hours of addition of T cells transduced with TFP, killing of the mesothelin or MUC16-expressing target cells may be essentially complete. Little or no killing may be observed with T cells transduced with a number of TFP constructs comprising other CD3 and TCR constructs. Similar results may be obtained with TFPs constructed with an alternative hinge region. Cytotoxicity against mesothelin-transduced target cells may be greater with TFP-transduced T cells than with CAR-transduced T cells.

The cytotoxic activity of TFP-transduced T cells may be dose-dependent with respect to the amount of virus (MOI) used for transduction. Increased killing of mesothelin-positive cells may be observed with increasing MOI of TFP lentivirus, further reinforcing the relationship between TFP transduction and cytotoxic activity.

### Example 7: Luciferase-Based Cytotoxicity Assay in Cells with High or Low Target Density

The luciferase-based cytotoxicity assay assesses the cytotoxicity of TFP T cells by indirectly measuring the luciferase enzymatic activity in the residual live target cells after co-culture.

A human tumor cell line, K562, is used as a target cell line for co-culture. K562 cells expressing no target (“DN”), MSLN (“MSLN+”), MUC16 (“MUC16+”), or both MSLN and MUC16 (“DP”) were generated by transduction with lentivirus encoding the human MSLN, human MUC16 ecto domain, or sequentially with both viruses. Target cells stably expressing desired target antigens were selected by application of antibiotics matched to the resistance gene encoded by the lentivirus. The target cells were further modified to overexpress firefly luciferase via transduction with firefly luciferase encoding lentivirus followed with antibiotic selection to generate stable cell line.

In a typical cytotoxicity assay, the target cells are plated at 5000 cells per well in 96-well plate. The TFP T or control cells were added to the target cells at a range of effector-to-target ratios. The mixture of cells was then cultured for 24 or 48 hours at 37° C. with 5% CO₂ before the luciferase enzymatic activity in the live target cells was measured by the Bright-Glo® Luciferase Assay System (Promega®, Catalog number E2610). The cells were spun into a pellet and resuspended in medium containing the luciferase substrate. The percentage of tumor cell killing was then calculated with the following formula: % Cytotoxicity=100%×[1−RLU (Tumor cells+T cells)/RLU (Tumor cells)].

### Example 8: Activation as Measured by CD69 or CD25 Upregulation on T Cells

Activation of the T cells expressing CAR and TFP Constructs are performed using MSLN+ or MUC16+ and MSLN− or MUC16− cells. As described above, Activated PBMCs are transduced with 50 MOI LVs for two consecutive days and expanded. Day 8 post transduction, co-cultures of PBMCs are set up with target cells at E:T, 1:1 ratio (0.2×10⁶ each cell type) in cytotoxicity medium (Phenol red-free RPMI1640 (Invitrogen®) plus 5% AB serum (Gemini Bio-products; 100-318). Cells overexpressing BCMA can be used as negative controls. 24 hours after the beginning of co-culturing, cells are harvested, washed with PBS three times and stained with Live/Dead Aqua for 30 min on ice. To block Fe receptors, human Fc block (BD) is added and incubated for 10 minutes at room temperature. Cells are subsequently stained with anti-CD3 APC (clone, UCHT1), anti-CD8 APCcy7(Clone SK1), anti-CD69-Alexa Fluor® 700 (clone FN50) from BD® Biosciences and anti-CD25-PE (Clone BC96, eBioscience®). Cells are washed twice and analyzed by BD LSRII-Fortessa®. Data are analyzed as above using FlowJo® analysis software (Tree star, Inc.).

A similar experiment cab be done using MSLN− or MUC16− cells and MSLN+ or MUC16+ cells in either non-transduced T cells or T cells transduced with positive control binders.

Activation of T cells may be similarly assessed by analysis of granzyme B production. T cells are cultured and expanded as described above, and intracellular staining for granzyme B is done according to the manufacturer's kit instructions (Gemini Bio-products; 100-318). Cells are harvested, washed with PBS three times and blocked with human Fc block for 10 min. Cells are stained for surface antigens with anti-CD3 APC (clone, UCHT1), and anti-CD8 APCcy7(Clone SK1) for 30 min at 4° C. Cells are then fixed with Fixation/Permeabilization solution (BD Cytofix/Cytoperm® Fixation/Permealbilzation kit cat #554714) for 20 min at 4 C, flowed by washing with BD Perm/Wash® buffer. Cells are subsequently stained with anti-Granzyme B Alexafluor700® (Clone GB11), washed with BD Perm/Wash buffer twice and resuspended in FACS buffer. Data is acquired on BD LSRII-Fortessa® and analyzed using FlowJo® (Tree star Inc.)

### Example 9: Comparative Quantitation of Cytokine Secretion by ELISA

Another measure of effector T cell activation and proliferation associated with the recognition of cells bearing cognate antigen is the production of effector cytokines such as interleukin-2 (IL-2) and interferon-gamma (IFN-γ).

granulocyte-macrophage colony-stimulating factor (GM-CSF) and tumor necrosis factor alpha (TNF-α).

Target-specific cytokine production including IL-2, IFN-γ, GM-CSF, and TNF-α by monospecific TFP T cells and dual-specific TFP T cells was measured from supernatants harvested 48 hours after the co-culture of T cells with various K562-based target cells using the U-PLEX® Biomarker Group I (hu) Assays (Meso Scale Diagnostics®, LLC, catalog number: K15067L-4).

Relative to non-transduced or control CAR-transduced T cells, T cells transduced with TFPs may produce higher levels of both IL-2 and IFN-γ when co-cultured with either cells that endogenously express mesothelin or MUC16 or mesothelin or MUC16-transduced cells. In contrast, co-culture with mesothelin or MUC16 negative cells or non-transduced cells, may result in little or no cytokine release from TFP-transduced T cells. Consistent with the previous cytotoxicity data, TFPs constructed with an alternative hinge region may generate similar results upon co-culture with mesothelin- or MUC16-bearing target cells.

### Example 10: Generation and Identification of Nanobodies Specific for Human MUC16 Peptide

### Materials and Methods

### Transformation, Recloning and Expression of V_{HH}ns Using Human MUC16 Peptide

### NFSPLARRVDRVAIYEEFLRMTRNGTQLQNFTLDRSSVLVDGYSPNRNEPLTGNSDLP (SEQ ID NO:92)

Transformation of Non-Suppressor Strain (e.g., WK6) with Recombinant pMECS GG

The nanobody gene cloned in pMECS GG vector contains PelB signal sequence at the N-terminus and HA tag and His₆ tag at the C-terminus (PelB leader-nanobody-HA-His₆). The PelB leader sequence directs the nanobody to the periplasmic space of the E. coli and the HA and His₆ tags can be used for the purification and detection of nanobody (e.g., in ELISA, western blot, etc.).

In pMECS GG vector, the His₆ tag is followed by an amber stop codon (TAG) and this amber stop codon is followed by gene III of M13 phage. In suppressor E. coli strains (e.g., TG1), the amber stop codon is read as glutamine and therefore the nanobody is expressed as fusion protein with protein III of the phage which allows the display of nanobody on the phage coat for panning. In non-suppressor E. coli strains (e. g., WK6), the amber stop codon is read as stop codon and therefore the resulting nanobody is not fused to protein III.

In order to express and purify nanobodies cloned in pMECS GG vector, pMECS GG vectors containing the gene of the nanobody of interest are prepared and used to transform a non-suppressor strain (e.g., WK6) with this plasmid. The nanobody of the resulting clone is sequenced using MP057 primer (5′-TTATGCTTCCGGCTCGTATG-3′ (SEQ ID NO:99)) to verify the identity of the clone. Retest antigen binding capacity by ELISA or any other appropriate assay. The non-suppressor strain (e.g., WK6) containing the recombinant pMECS GG vector with the nanobody gene can be used for the expression and purification of nanobody.

Recloning Nanobody Genes from pMECS GG to pHEN6c Vector

### Primer Sequences:

Primer A6E
(SEQ ID NO: 94)
(5′ GAT GTG CAG CTG CAG GAG TCT GGR GGA GG 3′).
Primer PMCF
(SEQ ID NO: 95)
(5′ CTA GTG CGG CCG CTG AGG AGA CGG TGA CCT GGG
T 3′).
Universal reverse primer
(SEQ ID NO: 96)
(5′ TCA CAC AGG AAA CAG CTA TGA C 3′).
Universal forward primer
(SEQ ID NO: 97)
(5 CGC CAG GGT TTT CCC AGT CAC GAC 3′).

The nanobody gene is amplified by PCR using E. coli containing recombinant pMECS GG harboring the nanobody gene as template and primers A6E and PMCF (about 30 cycles of PCR, each cycle consisting of 30 seconds at 94° C., 30 seconds at 55° C. and 45 seconds at 72° C., followed by 10 minutes extension at 72° C. at the end of PCR). A fragment of about 400 bp is amplified. The PCR product is then purified (e.g., by QiaQuick® PCR purification kit from Qiagen®) and digested overnight with PstI.

The PCR product is purified and digested with BstEII overnight (or with Eco91I from Fermentas Life Sciences®) The PCR product is purified as above and the pHEN6c vector is digested with PstI for 3 hours; the digested vector is purified as above and then digested with BstEII for 2 to 3 hours The digested vector is run on a 1% agarose gel, the vector band cut out of gel and purified (e.g., by QIAQuick gel extraction kit from Qiagen). The PCR product and the vector are ligated. Electrocompetent WK6 cells are transformed with the ligation reaction. Transformants are selected using LB/agar/ampicillin (100 μg/ml)/glucose (1-2%) plates.

### Expression and Purification of Nanobodies:

A freshly transformed WK6 colony is used to inoculate 10-20 ml of LB+ampicillin (100 μg/ml)+glucose (1%) and incubated at 37° C. overnight with shaking at 200-250 rpm. 1 ml of this pre-culture is added to 330 ml TB medium supplemented with 100 μg/ml Ampicillin, 2 mM MgCl₂ and 0.1% glucose and grow at 37° C. with shaking (200-250 rpm) until an OD₆₀₀ of 0.6-0.9 is reached. Nanobody expression is induced by addition of IPTG to final concentration of 1 mM and the culture is incubated at 28° C. with shaking overnight (about 16-18 hours; the OD₆₀₀ after overnight induction should ideally be between 25 and 30).

The culture is centrifuged for 8 minutes at 8000 rpm and the pellet resuspended from 1 liter culture in 12 ml TES (Sigma-Aldrich®) and shaken for 1 hour on ice. Per each 12 ml TES used, 18 ml TES/4 is added and further incubated on ice for an additional hour (with shaking) and then centrifuged for 30 min at 8000 rpm at 4° C. The supernatant contains proteins extracted from the periplasmic space.

### Purification by IMAC

His-select is equilibrated with PBS: per periplasmic extract derived from 1 liter culture, 1 ml Resin is added (about 2 ml His-select solution) to a 50 ml falcon tube, PBS is added to a final volume of 50 ml and mixed and then centrifuged at 2000 rpm for 2 min. and the supernatant discarded. The resin is washed twice with PBS and then the periplasmic extract is added and incubated for 30 minutes to 1 hour at room temperature with gentle shaking (longer incubation times may result in non-specific binding).

The sample is loaded onto a PD-10 column with a filter at the bottom (GE healthcare, cat. No. 17-0435-01) and washed with 50 to 100 ml PBS (50-100 ml PBS per 1 ml resin used). Elution is performed 3 times, each time with 1 ml PBS/0.5 M imidazole per 1 ml resin used, and the combined eluent is dialyzed overnight at 4° C. against PBS (cutoff 3500 Daltons) to remove imidazole.

The amount of protein can be estimated at this point by OD₂₈₀ measurement of eluted sample. Extinction coefficient of each clone can be determined by ProtParam tool under primary structure analysis at the Expasy proteomics server. Further purification of nanobodies can be achieved by different methods. For example, the sample may be concentrated (Vivaspin® 5000 MW cutoff, Vivascience®) by centrifuging at 2000 rpm at 4° C. till an appropriate volume for loading on a Superdex® 75 16/60 is obtained (max. 4 ml). The concentrated sample is then loaded onto a Superdex 75 16/60 column equilibrated with PBS. Peak fractions are pooled and the sample is measured at OD₂₈₀ for quantification. Aliquots are stored at −20° C. at a concentration of about 1 mg/ml.

### Immunization

A llama was subcutaneously injected on days 0, 7, 14, 21, 28 and 35, with human MUC16 peptide (hMUC16) conjugated to KLH (NFSPLARRVDRVAIYEEFLRMTRNGTQLQNFTLDRSSVLVDGYSPNRNEPLTGNSDLP—C-KLH) (SEQ ID NO:93) and/or human MUC16 peptide biotinylated at C-terminus (NFSPLARRVDRVAIYEEFLRMTRNGTQLQNFTLDRSSVLVDGYSPNRNEPLTGNSDLP—C-Biotin) and/or human MUC16 peptide biotinylated at N-terminus (Biotin-NF SPLARRVDRVAIYEEFLRMTRNGTQLQNFTLDRSSVLVDGYSPNRNEPLTGNSDLP. The biotinylated peptides were mixed with neutralite avidin before injections. The adjuvant used was GERBU adjuvant P (GERBU Biotechnik GmbH. On day 40, about 100 ml anticoagulated blood was collected from the llama for lymphocyte preparation.

### Construction of a VHH Library

A VHH library was constructed from the llama lymphocytes to screen for the presence of antigen-specific nanobodies. To this end, total RNA from peripheral blood lymphocytes was used as template for first strand cDNA synthesis with an oligo(dT) primer. Using this cDNA, the VHH encoding sequences were amplified by PCR, digested with SAPI, and cloned into the SAPI sites of the phagemid vector pMECS-GG. The VHH library thus obtained was called Core 93GG. The library consisted of about 10⁸ independent transformants, with about 87% of transformants harboring the vector with the right insert size.

### Isolation of Human MUC16 Peptide-Specific Nanobodies

The Core 93GG library was panned on hMUC16 peptide NFSPLARRVDRVAIYEEFLRMTRNGTQLQNFTLDRSSVLVDGYSPNRNEPLTGNSDLP (SEQ ID NO:92) biotinylated either at C- or N-terminus (bio-hMUC16) for 4 rounds. The bio-hMUC16 peptide was allowed to interact with streptavidin coated plates after which phages from the library were added to the plate. The enrichment for antigen-specific phages was assessed after each round of panning by comparing the number of phagemid particles eluted from antigen-coated wells with the number of phagemid particles eluted from negative control wells (coated with streptavidin and blocked but containing no peptide). These experiments suggested that the phage population was enriched for antigen-specific phages about 2-fold after the 2^{nd} round. No enrichment was observed after the 1^{st}, 3^{rd} and 4^{th} round. In total, 380 colonies (190 from round 3, 190 from round 4) were randomly selected and analyzed by ELISA for the presence of antigen-specific nanobodies in their periplasmic extracts (ELISA using crude periplasmic extracts including soluble nanobodies). The peptides used for ELISA screening were the same as the ones used for panning, using blocked streptavidin-coated wells without peptide as negative control. Out of these 380 colonies, 34 colonies scored positive in this assay. Based on sequence data of the positive colonies, 6 different full length nanobodies were distinguished, belonging to 2 different CDR3 groups (B-cell lineages) (see Excel file). Nanobodies belonging to the same CDR3 group (same B-cell lineage) are very similar and their amino acid sequences suggest that they are from clonally-related B-cells resulting from somatic hypermutation or from the same B-cell but diversified due to RT and/or PCR error during library construction. Nanobodies belonging to the same CDR3 group recognize the same epitope but their other characteristics (e.g. affinity, potency, stability, expression yield, etc.) can be different. Clones from these pannings bear the following code in their name: MU.

Flow Cytometry Analysis of hMUC16 Peptide-Specific Nanobodies

### Nanobodies and Cells

Periplasmic extracts were generated for each anti-hMUC16-peptide Nb in the same way as was done for the initial ELISA screening described above. Cells from each cell-line (SKOV3 Muc16 Luc, OVCAR 3 Muc16 Luc, Expi-293 and Jurkat) were thawed, washed and counted. The periplasmic extract from each Nb clone was incubated with about 2×10⁵ cells. After washing, the cells were incubated with a mix of mouse anti-HA tag antibody and anti-mouse-PE. After another wash, To-pro® (Thermo Fisher Scientific®) was added to each sample as live/dead stain and the cells were analyzed on a flow cytometer. As a positive control Mab, human anti-Muc16-4h11 (+anti-human IgG-PE+To-pro), was used on the SKOV3 Muc16 Luc and OVCAR 3 Muc16 Luc cells. As negative controls, we used for each cell line: a sample with an irrelevant Nb (BCII10—bacterial β lactamase specific), a sample with all detection Mabs, a sample with the secondary anti-mouse-PE Mab alone and a sample with cells alone (with and without To-pro).

### Example 11: Flow Cytometry-Based MSLN- and MUC16-Specific TFP Detection in the Jurkat Human T Cell Line

The expression of a MSLN and MUC16 dual-specificity TFP was evaluated first in the Jurkat human T cell line using flow cytometry. Lentivirus preparations encoding the MSLN-specific TFP, MUC16-specific TFP or dual-specific TFP (MSLN TFP and MUC16 TFP in a single lentiviral vector linked by a T2A sequence) were used to transduce the Jurkat cells.

Forty-eight hours after lentivirus transduction, transduced Jurkat cells and non-transduced (NT) control cells were harvested and analyzed for the surface expression of MSLN− and MUC16-specific TFPs. MSLN-specific TFPs were detected by the Fc_MSLN, human Mesothelin/MSLN (296-580) protein with a Fc tag (AcroBiosystems, catalog number: MSN-H526x). The protein was labeled with Zenon™ Allophycocyanin Human IgG Labelling Kit (ThermoFisher Scientific, catalog number: Z25451) and used at 1 μg/sample for staining.

The MUC16-specific TFPs were detected by a MUC16-biotin peptide (UniProtKB: Q8WXI7, aa 14319-14438, synthesized at New England Peptide), followed with streptavidin-PE (BD Bioscience, catalog number: 554061). The MUC16 peptide was used at 40 picomole per sample. All Jurkat cells (NT, MSLN TFP, MUC16 TFP, dual specific TFP) were stained first with labelled Fc_MSLN and MUC16-biotin, concurrently, then stained with streptavidin-PE.

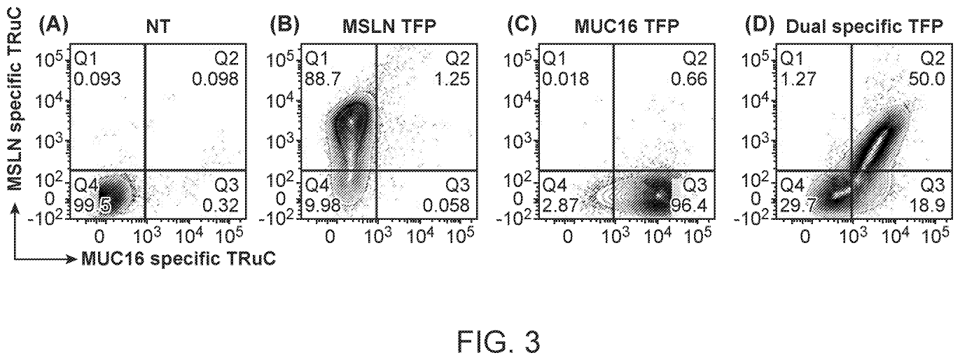

Expression of MSLN specific TFP, but not MUC16 TFP, was detected on Jurkat cells transduced with lentivirus encoding MSLN TFP (FIG. 3B). In addition, MUC16 TFP, but not MSLN TFP, was detected on Jurkat cells transduced with lentivirus encoding MUC16 TFP (FIG. 3C). For Jurkat cells transduced with lentivirus encoding dual-specific TFP, both MSLN TFP and MUC16 TFP were detected on the surface of the same population of transduced Jurkat cells (FIG. 3D. No detection of MSLN TFP or MUC16 TFP was observed for NT Jurkat cells (FIG. 3A).

### Example 13: Target-Specific Cytokine Production by Dual-Specific TFP Jurkat Cells

Target-specific cytokine production by monospecific TFP Jurkat cells and dual-specific TFP Jurkat cells was measured in supernatants harvested 24 hours after the co-culture of Jurkat cells with various K562-based target cells, expressing no target (“DN”), MSLN (“MSLN+”), MUC16 (“MUC16+”), or both MSLN and MUC16 (“DP”). The level of human IL-2 in the supernatants was analyzed using Meso Scale Discovery Technology (MesoScale Diagnostic, LLC), with U-PLEX Biomarker Group I (hu) Assays (Catalog number: K15067L-4).

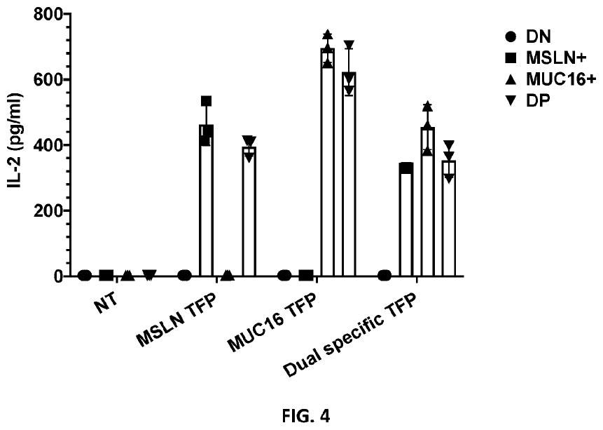

NT Jurkat cells did not produce any detectable IL-2 in co-culture with any target tumor cells, regardless of target expression (FIG. 4). Monospecific TFP Jurkat cells produced IL-2 only in co-culture with target cells expressing matched targets (i.e., MSLN TFP Jurkat cells co-cultured with MSLN-expressing or overexpressing K562 cells and MUC16 TFP Jurkat cells co-cultured with MUC16-expressing or overexpressing K562 cells). MSLN TFP Jurkat cells produced IL-2 in co-culture with MSLN+ target cells or DP target cells, but not with DN or MUC16+ target cells. MUC16 TFP Jurkat cells produced IL-2 in co-culture with MUC16+ target cells or DP target cells, but not with DN or MSLN+ target cells. Dual-specific TFP Jurkat cells produced IL-2 in response to target cells expressing either of the targets, MSLN only (MSLN+), MUC16 only (MUC16+), or both targets (DP), demonstrating broader reactivity than both monospecific TFP Jurkat cells (FIG. 4). The lack of IL-2 production in co-culture with target cells expressing no target (DN) confirmed the specificity of the dual-specific TFP.

### Example 14: Flow Cytometry Based MSLN and MUC16 Dual-Specific TFP Detection in Primary Human T Cells

NT, MSLN TFP, MUC16 TFP and dual-specific TFP T cells were generated from healthy donor human primary T cells by transduction with a lentivirus encoding mono or dual-specific TFPs. The T cells were purified from healthy donor PBMCs and activated on day 0 by MACS GMP T Cell TransAct® (Miltenyi® Biotech, catalog number: 130-019-011), in the presence of Human IL-7, premium grade (Miltenyi Biotech, catalog number: 130-095-364) and Human IL-15, premium grade (Miltenyi Biotech, catalog number: 130-095-766). On day 1, activated T cells were transduced with lentivirus and the cells were expanded for 10 days by supplementing fresh medium every 2 days.

On day 10, T cells were harvested and stained by flow cytometry with Fc_MSLN and MUC16-biotin peptide, as described above, to determine surface expression of mono or dual-specific TFPs. MonoRab® Rabbit Anti-Camelid VHH Antibody [iFluor488] (GenScript®, catalog number: A01862) was used in addition to the ligands to detect the TFPs.

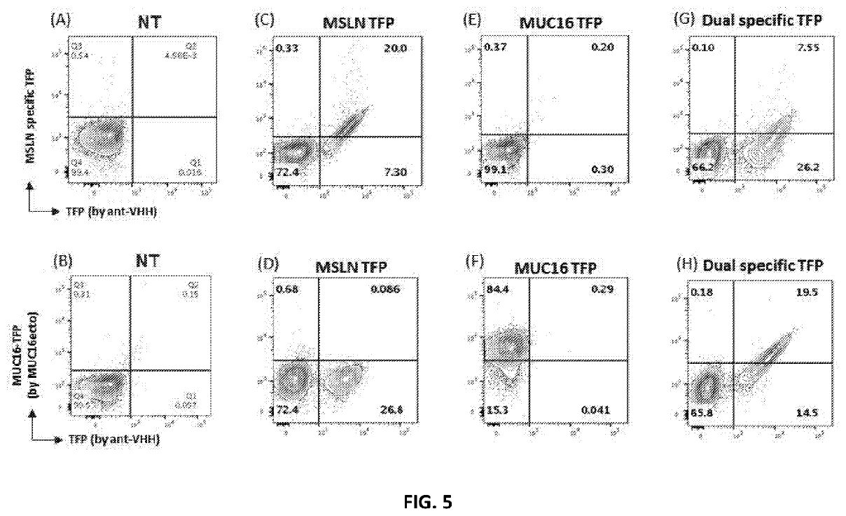

Similar to results seen for assays using Jurkat cells, expression of MSLN specific TFPs (FIG. 5C), but not MUC16 TFPs (FIG. 5D), were detected for MSLN TFP T cells; in addition, MUC16 TFPs (FIG. 5F), but not MSLN TFPs (FIG. 5E), were detected for MUC16 TFP T cells. For dual-specific TFP T cells, both MSLN TFPs and MUC16 TFPs were detected on the surface of the transduced cells (FIGS. 5G and 5H). No detection of MSLN TFP or MUC16 TFP was observed for NT T cells (FIGS. 5A and 5B).

### Example 15: Target-Specific Tumor Cell Killing by Dual-Specific TFP T Cells

Target-specific tumor cell killing by mono specific and dual specific TFP T cells was evaluated using an in vitro cytotoxicity assay using primary human T cells prepared according to Example 14. Tumor cell lines expressing no target (DN), MSLN (MSLN+), MUC16 (MUC16+), or both MSLN and MUC16 (DP) (as described in Example 13) were stably transduced to express firefly luciferase as the reporter. After forty-eight hours of co-culture with NT or TFP T cells, the luciferase activity of the co-cultured cells was determined with the Bright-Glo® Luciferase Assay System (Promega®, Catalogue number E2610) as a marker for viable tumor cells. The percentage of tumor cell killing was then calculated with the following formula: % Cytotoxicity=100%×[1−RLU (Tumor cells+T cells)/RLU (Tumor cells)].

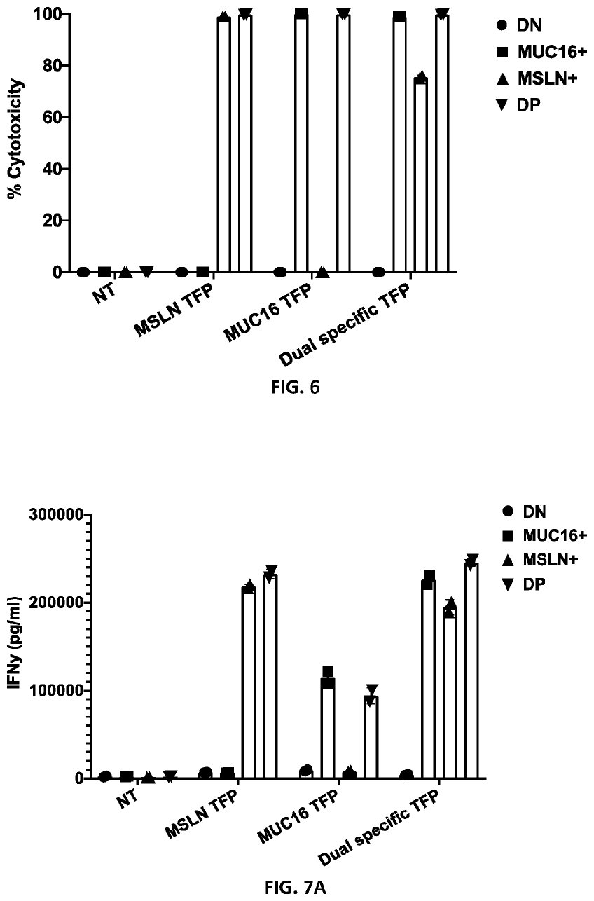

As expected, NT T cells showed no detectable killing against any of the target cells (FIG. 6). Monospecific TFP T cells only killed target cells expressing matched targets. MSLN TFP T cells dramatically killed MSLN+ target cells or DP target cells, but not DN or MUC16+ target cells. MUC16 TFP T cells completely killed MUC16+ target cells or DP target cells, but not DN or MSLN+ target cells. Dual-specific TFP T cells significantly killed target cells expressing either of the targets, MSLN only (MSLN+), MUC16 only (MUC16+), or both targets (DP), demonstrated a broader range of reactivity than both monospecific TFP T cells (FIG. 6). The lack of killing against target cells expressing no target (DN) confirmed the specificity of the dual-specific TFP T cells.

### Example 17: Target-Specific Cytokine Production by Dual-Specific TFP T Cells

Primary human T cells were prepared and transduced by the methods described in previous Examples. Target-specific cytokine production including IFN-γ, GM-CSF, and TNF-α by monospecific TFP T cells and dual-specific TFP T cells was measured from supernatants harvested 48 hours after the co-culture of T cells with various K562-based target cells using the U-PLEX® Biomarker Group I (hu) Assays (Meso Scale Diagnostics®, LLC, catalog number: K15067L-4).

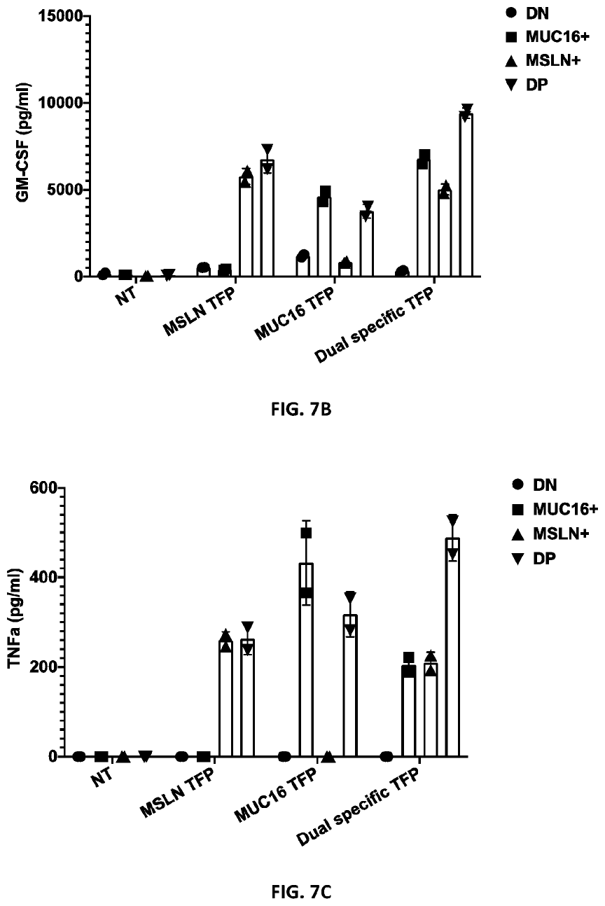

All TFP T cells produced significant amounts of IFN-γ when co-cultured with tumor cells expressing the matched targets (FIG. 7A). Consistent with the lack of killing against tumor cells with unmatched target expression, and their specificity, no cytokine production was observed for MSLN TFP T cells cultured with MUC16+ target cells, or for MUC16 TFP T cells cultured with MSLN+ target cells. Dual-specific TFP T cells, on the contrary, were observed to have broader reactivity than either of the monospecific TFP T cells with significant IFN-γ production observed following co-culture with MSLN+, MUC16+ or DP target cells (FIG. 7A).

Noticeable production of GM-CSF (FIG. 7B) and TNF-α (FIG. 7C) was observed for monospecific TFP T cells and dual-specific TFP T cells, with a similar reactivity pattern against the tumor cells. MSLN TFP and MUC16 TFP T cells only produced cytokines when co-cultured with target-matched tumor cells, but not with target-mismatched cells. Dual-specific TFP T cells responded to target cells expressing either or both targets.

### Example 18: Clinical Studies

Patients with unresectable ovarian cancer with relapsed or refractory disease will be enrolled for clinical studies of T cells expressing MSLN-MUC16-TFPs. The initial study will explore the safety profile of T cells expressing MSLN-MUC16-TFPs and will explore cell kinetics and pharmacodynamics outcomes. Those results will inform the selection of dosages for further studies, which will then be administered to a larger cohort of patients with unresectable ovarian cancer to define the efficacy profile of T cells expressing MSLN-MUC16-TFPs.

### Example 19: CD107a Exposure by Flow Cytometry

An additional assay for T cell activation is surface expression of CD107a, a lysosomal associated membrane protein (LAMP-1) that is located in the membrane of cytoplasmic cytolytic granules in resting cells. Degranulation of effector T cells, a prerequisite for cytolytic activity, results in mobilization of CD107a to the cell surface following activation-induced granule exocytosis. Thus, CD107a exposure provides an additional measure of T cell activation, in addition to cytokine production, that correlates closely with cytotoxicity.

Target and effector cells are separately washed and re-suspended in cytotoxicity medium (RPMI+5% human AB serum+1% antibiotic antimycotic). The assay is performed by combining 2×10⁵ effectors cells with 2×10⁵ target cells in a 100 μL final volume in U-bottom 96-well plates (Corning), in the presence of 0.5 μL/well of PE/Cy7-labelled anti-human CD107a (LAMP-1) antibody (Clone-H4A3, BD® Biosciences). The cultures are then incubated for an hour at 37° C., 5% CO₂. Immediately following this incubation, 10 μL of a 1:10 dilution of the secretion inhibitor monensin (1000× solution, BD GolgiStop™) is carefully added to each well without disturbing the cells. The plates are then incubated for a further 2.5 hours at 37° C., 5% CO₂. Following this incubation, the cells are stained with APC anti-human CD3 antibody (Clone-UCHT1, BD Biosciences), PerCP/Cy5.5 anti-human CD8 antibody (Clone-SK1, BD Biosciences) and Pacific Blue anti-human CD4 antibody (Clone-RPA-T4, BD Biosciences) and then incubated for 30 minutes at 37° C., 5% CO₂. The cells are then washed 2× with FACS buffer (and resuspended in 100 μL FACS buffer and 100 μl IC fix buffer prior to analysis.

Exposure of CD107a on the surface of T cells is detected by flow cytometry. Flow cytometry is performed with a LSRFortessa® X20 (BD Biosciences) and analysis of flow cytometric data is performed using FlowJo® software (Treestar, Inc. Ashland, Oreg.). The percentage of CD8+ effector cells, within the CD3 gate, that are CD107+ve is determined for each effector/target cell culture.

Consistent with the previous cytotoxicity and cytokine data, co-culture of tumor-associated antigen-expressing target cells with effector T cells transduced with anti-tumor-associated antigen-28ζ CAR may induce an increase in surface CD107a expression relative to effectors incubated with tumor-associated antigen negative target cells. In comparison, under the same conditions, anti-tumor-associated antigen-CD3ε LL or anti-tumor-associated antigen-CD3γ LL TFP-expressing effectors may exhibit a 5 to 7-fold induction of CD107a expression. Anti-tumor-associated antigen TFPs constructed with an alternative hinge region may generate similar results upon co-culture with tumor-associated antigen-bearing target cells.

### Example 20: In Vivo Mouse Efficacy Studies

To assess the ability of effector T cells transduced with anti-tumor-associated antigen TFPs to achieve anti-tumor responses in vivo, effector T cells transduced with either anti-tumor-associated antigen-28ζ CAR, anti-tumor-associated antigen-CD3ε TFP or anti-tumor-associated antigen-CD3γ TFP are adoptively transferred into NOD/SCID/IL-2Rγ−/− (NSG-JAX) mice that had previously been inoculated with tumor-associated antigen+human cancer cell lines.

Female NOD/SCID/IL-2Rγ−/− (NSG-JAX) mice, at least 6 weeks of age prior to the start of the study, are obtained from The Jackson Laboratory (stock number 005557) and acclimated for 3 days before experimental use. Human tumor-associated antigen-expressing cell lines for inoculation are maintained in log-phase culture prior to harvesting and counting with trypan blue to determine a viable cell count. On the day of tumor challenge, the cells are centrifuged at 300 g for 5 minutes and re-suspended in pre-warmed sterile PBS at either 0.5-1×10⁶ cells/100 μL. T cells for adoptive transfer, either non-transduced or transduced with anti-tumor-associated antigen-28ζ CAR, anti-tumor-associated antigen-CD3ε TFP or anti-CD3γ TFP constructs are prepared. On day 0 of the study, 10 animals per experimental group are challenged intravenously with 0.5-1×10⁶ tumor-associated antigen-expressing cells. 3 days later, 5×10⁶ of effector T cell populations are intravenously transferred to each animal in 100 μL of sterile PBS. Detailed clinical observations on the animals are recorded daily until euthanasia. Body weight measurements are made on all animals weekly until death or euthanasia. All animals are euthanized 35 days after adoptive transfer of test and control articles. Any animals appearing moribund during the study are euthanized at the discretion of the study director in consultation with a veterinarian.

Relative to non-transduced T cells, adoptive transfer of T cell transduced with either anti-tumor-associated antigen-28ζ CAR, anti-tumor-associated antigen-CD3ε TFP or anti-tumor-associated antigen-CD3γ TFP may prolong survival mesothelin-expressing cell line tumor-bearing mice, and may indicate that both anti-tumor-associated antigen CAR and TFP-transduced T cells are capable of mediating target cell killing with corresponding increased survival in these mouse models. Collectively, these data may indicate that TFPs represent an alternative platform for engineering chimeric receptors that demonstrate superior antigen-specific killing to first generation CARs both in vitro and in vivo

TABLE 2
Exemplary sequences

| SEQ | | |
|---|---|---|
| ID | | |
| NO. | Name | Sequence |
| 1. | (G₄S)₃ Linker | GGGGSGGGGSGGGGSLE |
| 2. | (G₄S)₄ Linker | GGGSGGGGSGGGGSGGGGSLE |
| 3. | human CD3-ϵ | MQSGTHWRVLGLCLLSVGVWGQDGNEEMGGITQTPYKVSISGT |
| | | TVILTCPQYPGSEILWQHNDKNIGGDEDDKNIGSDEDHLSLKEFS |
| | | ELEQSGYYVCYPRGSKPEDANFYLYLRARVCENCMEMDVMSV |
| | | ATIVIVDICITGGLLLLVYYWSKNRKAKAKPVTRGAGAGGRQR |
| | | GQNKERPPPVPNPDYEPIRKGQRDLYSGLNQRRI |
| 4. | human CD3-γ | MEQGKGLAVLILAIILLQGTLAQSIKGNHLVKVYDYQEDGSVLL |
| | | TCDAEAKNITWFKDGKMIGFLTEDKKKWNLGSNAKDPRGMYQ |
| | | CKGSQNKSKPLQVYYRMCQNCIELNAATISGFLFAEIVSIFVLAV |
| | | GVYFIAGQDGVRQSRASDKQTLLPNDQLYQPLKDREDDQYSHL |
| | | QGNQLRRN |
| 5. | human CD3-δ | MEHSTFLSGLVLATLLSQVSPFKIPIEELEDRVFVNCNTSITWVEG |
| | | TVGTLLSDITRLDLGKRILDPRGIYRCNGTDIYKDKESTVQVHYR |
| | | MCQSCVELDPATVAGIIVTDVIATLLLALGVFCFAGHETGRLSG |
| | | AADTQALLRNDQVYQPLRDRDDAQYSHLGGNWARNKS |
| 6. | human CD3-ζ | MKWKALFTAAILQAQLPITEAQSFGLLDPKLCYLLDGILFIYGVI |
| | | LTALFLRVKFSRSADAPAYQQGQNQLYNELNLGRREEYDVLDK |
| | | RRGRDPEMGGKPQRRKNPQEGLYNELQKDKMAEAYSEIGMKG |
| | | ERRRGKGHDGLYQGLSTATKDTYDALHMQALPPR |
| 7. | human TCR | MAGTWLLLLLALGCPALPTGVGGTPFPSLAPPIMLLVDGKQQM |
| | α-chain | VVVCLVLDVAPPGLDSPIWFSAGNGSALDAFTYGPSPATDGTW |
| | | TNLAHLSLPSEELASWEPLVCHTGPGAEGHSRSTQPMHLSGEAS |
| | | TARTCPQEPLRGTPGGALWLGVLRLLLFKLLLFDLLLTCSCLCD |
| | | PAGPLPSPATTTRLRALGSHRLHPATETGGREATSSPRPQPRDRR |
| | | WGDTPPGRKPGSPVWGEGSYLSSYPTCPAQAWCSRSALRAPSSS |
| | | LGAFFAGDLPPPLQAGA |
| 8. | human TCR | PNIQNPDPAVYQLRDSKSSDKSVCLFTDFDSQTNVSQSKDSDVYI |
| | α-chain C | TDKTVLDMRSMDFKSNSAVAWSNKSDFACANAFNNSIIPEDTFF |
| | region | PSPESSCDVKLVEKSFETDTNLNFQNLSVIGFRILLLKVAGFNLL |
| | | MTLRLWSS |
| 9. | human TCR | MAMLLGASVLILWLQPDWVNSQQKNDDQQVKQNSPSLSVQEG |
| | α-chain V | RISILNCDYTNSMFDYFLWYKKYPAEGPTFLISISSIKDKNEDGRF |
| | region CTL- | TVFLNKSAKHLSLHIVPSQPGDSAVYFCAAKGAGTASKLTFGTG |
| | L17 | TRLQVTL |
| 10 | human TCR | EDLNKVFPPEVAVFEPSEAEISHTQKATLVCLATGFFPDHVELSW |
| | β-chain C | WVNGKEVHSGVSTDPQPLKEQPALNDSRYCLSSRLRVSATFWQ |
| | region | NPRNHFRCQVQFYGLSENDEWTQDRAKPVTQIVSAEAWGRAD |
| | | CGFTSVSYQQGVLSATILYEILLGKATLYAVLVSALVLMAMVK |
| | | RKDF |
| 11 | human TCR | MGTSLLCWMALCLLGADHADTGVSQNPRHNITKRGQNVTFRC |
| | β-chain V | DPISEHNRLYWYRQTLGQGPEFLTYFQNEAQLEKSRLLSDRFSA |
| | region CTL- | ERPKGSFSTLEIQRTEQGDSAMYLCASSLAGLNQPQHFGDGTRL |
| | L17 | SIL |
| 12 | human TCR | MDSWTFCCVSLCILVAKHTDAGVIQSPRHEVTEMGQEVTLRCK |
| | β-chain V | PISGHNSLFWYRQTMMRGLELLIYFNNNVPIDDSGMPEDRFSAK |
| | region YT35 | MPNASFSTLKIQPSEPRDSAVYFCASSFSTCSANYGYTFGSGTRL |
| | | TVV |
| 13 | Nucleic acid | caggtgcagctgcaggagtctgggggaggattggtgcaggctgggggctctctgagactctcctgtg |
| | sequence | cagcctctggacgcaccgtcagtagcttgttcatgggctggttccgccaagctccagggaaggagcg |
| | encoding | tgaacttgtagcagccattagccggtatagtctatatacatactatgcagactccgtgaagggccgattc |
| | single domain | accatctccgcagacaacgccaagaacgcggtatatctgcaaatgaacagcctgaaacctgaggac |
| | anti-MUC16 | acggccgtttattactgtgcatcaaagttggaatatacttctaatgactatgactcctggggccagggga |
| | binder 1 | cccaggtcaccgtctcctca |
| | (SD1) | |
| 14 | single domain | QVQLQESGGGLVQAGGSLRLSCAASGRTVSSLFMGWFRQAPG |
| | anti-MUC16 | KERELVAAISRYSLYTYYADSVKGRFTISADNAKNAVYLQMNS |
| | binder | LKPEDTAVYYCASKLEYTSNDYDSWGQGTQVTVSS |
| | R3MU4 | |
| 15 | R3MU4CDR1 | GRTVSSLF |
| 16 | R3MU4 | ISRYSLYT |
| | CDR2 | |
| 17 | R3MU4 | ASKLEYTSNDYDS |
| | CDR3 | |
| 18 | Nucleic acid | caggtgcagctgcaggagtctgggggaggattggtgcaggctggggactctctgagactctcctgtg |
| | sequence | cagcctctggacgcgccgtcagtagcttgttcatgggctggttccgccgagctccagggaaggagcg |
| | encoding | tgaacttgtagcagccattagccggtatagtctatatacatactatgcagactccgtgaagggccgattc |
| | single domain | accatctccgcagacaacgccaagaacgcggtatatctgcaaatgaacagcctaaaacctgaggaca |
| | anti-MUC16 | cggccgtttattactgtgcatcaaagttggaatatacttctaatgactatgactcctggggccaggggac |
| | R3MU29 | ccaggtcaccgtctcctca |
| 19 | Single domain | QVQLQESGGGLVQAGDSLRLSCAASGRAVSSLFMGWFRRAPG |
| | anti-MUC16 | KERELVAAISRYSLYTYYADSVKGRFTISADNAKNAVYLQMNS |
| | R3MU29 | LKPEDTAVYYCASKLEYTSNDYDSWGQGTQVTVSS |
| 20 | R3MU29 | GRAVSSLF |
| | CDR1 | |
| 21 | R3MU29 | ISRYSLYT |
| | CDR2 | |
| 22 | R3MU29 | ASKLEYTSNDYDS |
| | CDR3 | |
| 23 | Nucleic acid | caggtgcagctgcaggagtctgggggaggattggtgcaggctggggactctctgagactctcctgtg |
| | sequence | cagcctctggacgcaccgtcagtagcttgttcatggggtggttccgccgagctccagggaaggagcg |
| | encoding | tgaacttgtagcagccattagccggtatagtctatatacatactatgcagactccgtgaagggccgattc |
| | single domain | accatctccgcagacaacgccaagaacgcggtatatctgcaaatgaacagcctgaaacctgaggac |
| | anti-MUC16 | acggccgtttattactgtgcatcaaagttggaatatacttctaatgactatgactcctggggccagggga |
| | R3MU63 | cccaggtcaccgtctcctca |
| 24 | Single domain | QVQLQESGGGLVQAGDSLRLSCAASGRTVSSLFMGWFRRAPG |
| | anti-MUC16 | KERELVAAISRYSLYTYYADSVKGRFTISADNAKNAVYLQMNS |
| | R3MU63 | LKPEDTAVYYCASKLEYTSNDYDSWGQGTQVTVSS |
| 25 | R3MU63 | GRTVSSLF |
| | CDR1 | |
| 26 | R3MU63 | ISRYSLYT |
| | CDR2 | |
| 27 | R3MU63 | ASKLEYTSNDYDS |
| | CDR3 | |
| 28 | Nucleic acid | caggtgcagctgcaggagtctgggggaggtttggtgcagcctggggattctatgagactctcctgtgc |
| | sequence | agccgagggggactctttggatggttatgtagtaggttggttccgccaggccccagggaaggagcgc |
| | encoding | cagggggtctcaagtattagtggcgatggcagtatgcgatacgttgctgactccgtgaaggggcgatt |
| | single domain | caccatctcccgagacaacgccaagaacacggtgtatctgcaaatgatcgacctgaaacctgaggac |
| | anti-MUC16 | acaggcgtttattactgtgcagcagacccacccacttgggactactggggtcaggggacccaggtca |
| | R3MU119 | ccgtctcctca |
| 29 | Single domain | QVQLQESGGGLVQPGDSMRLSCAAEGDSLDGYVVGWFRQAPG |
| | anti-MUC16 | KERQGVSSISGDGSMRYVADSVKGRFTISRDNAKNTVYLQMID |
| | R3MU119 | LKPEDTGVYYCAADPPTWDYWGQGTQVTVSS |
| 30 | R3MU119 | GDSLDGYV |
| | CDR1 | |
| 31 | R3MU119 | ISGDGSMR |
| | CDR2 | |
| 32 | R3MU119 | AADPPTWDY |
| | CDR3 | |
| 33 | Nucleic acid | caggtgcagctgcaggagtctgggggaggcttggtgcagcctggggggtactgagactctcctgtg |
| | sequence | cagcctctggacgcaccgtcagtagcttgttcatgggctggttccgccgagctccagggaaggagcg |
| | encoding | tgaacttgtagcagccattagccggtatagtctatatacatactatgcagactccgtgaagggccgattc |
| | single domain | accatctccgcagacaacgccaagaacgcggtatatctgcaaatgaacagcctgaaacctgaggac |
| | anti-MUC16 | acggccgtttattactgtgcatcaaagttggaatatacttctaatgactatgactcctggggccagggga |
| | R3MU150 | cccaggtcaccgtctcctca |
| 34 | Single domain | QVQLQESGGGLVQPGGSLRLSCAASGRTVSSLFMGWFRRAPGK |
| | anti-MUC16 | ERELVAAISRYSLYTYYADSVKGRFTISADNAKNAVYLQMNSL |
| | R3MU150 | KPEDTAVYYCASKLEYTSNDYDSWGQGTQVTVSS |
| 35 | R3MU150 | GRTVSSLF |
| | CDR1 | |
| 36 | R3MU150 | ISRYSLYT |
| | CDR2 | |
| 37 | R3MU150 | ASKLEYTSNDYDS |
| | CDR3 | |
| 38 | Nucleic acid | caggtgcagctgcaggagtctgggggaggattggtgcaggctggggagtctctgagactctcctgtg |
| | sequence | cagcctctggacgcaccgtcagtagcttgttcatgggctggttccgccgagctccagggaaggagcg |
| | encoding | tgaacttgtagcagccattagccggtatagtctatatacatactatgcagactccgtgaagggccgattc |
| | single domain | accatctccgcagacaacgccaagaacgcggtatatctgcaaatgaacagcctgaaacctgaggac |
| | anti-MUC16 | acggccgtttattactgtgcatcaaagttggaatatacttctaatgactatgactcctggggccagggga |
| | R3MU147 | cccaggtcaccgtctcctca |
| 39 | Single domain | QVQLQESGGGLVQAGESLRLSCAASGRTVSSLFMGWFRRAPG |
| | anti-MUC16 | KERELVAAISRYSLYTYYADSVKGRFTISADNAKNAVYLQMNS |
| | R3MU147 | LKPEDTAVYYCASKLEYTSNDYDSWGQGTQVTVSS |
| 40 | R3MU147 | GRTVSSLF |
| | CDR1 | |
| 41 | R3MU147 | ISRYSLYT |
| | CDR2 | |
| 42 | R3MU147 | ASKLEYTSNDYDS |
| | CDR3 | |
| 43 | R3MU29h15 | EVQLVESGGGLVQPGGSLRLSCAASGRAVSSLFMGWVRQAPG |
| | (98.9% | KGLEWVSAISRYSLYTYYADSVKGRFTISRDNAKNTLYLQMN |
| | human) | SLRPEDTAVYYCASKLEYTSNDYDSWGQGTLVTVSS |
| 44 | R3MU29h14 | EVQLVESGGGLVQPGGSLRLSCAASGRAVSSLFMGWFRQAPG |
| | (97.8% | KGLEWVSAISRYSLYTYYADSVKGRFTISRDNAKNTLYLQMN |
| | human) | SLRPEDTAVYYCASKLEYTSNDYDSWGQGTLVTVSS |
| 45 | R3MU29h13 | EVQLVESGGGLVQPGGSLRLSCAASGRAVSSLFMGWFRQAPG |
| | (96.7% | KGLELVSAISRYSLYTYYADSVKGRFTISRDNAKNTLYLQMN |
| | human) | SLRPEDTAVYYCASKLEYTSNDYDSWGQGTLVTVSS |
| 46 | R3MU4h13 | EVQLVESGGGLVQPGGSLRLSCAASGRTVSSLFMGWVRQAPG |
| | (98.9% | KGLEWVSAISRYSLYTYYADSVKGRFTISRDNAKNTLYLQMN |
| | human | SLRPEDTAVYYCASKLEYTSNDYDSWGQGTLVTVSS |
| 47 | R3MU4h12 | EVQLVESGGGLVQPGGSLRLSCAASGRTVSSLFMGWFRQAPG |
| | (97.8% | KGLEWVSAISRYSLYTYYADSVKGRFTISRDNAKNTLYLQMN |
| | human) | SLRPEDTAVYYCASKLEYTSNDYDSWGQGTLVTVSS |
| 48 | R3MU4h11 | EVQLVESGGGLVQPGGSLRLSCAASGRTVSSLFMGWFRQAPG |
| | (96.7% | KGLELVSAISRYSLYTYYADSVKGRFTISRDNAKNTLYLQMN |
| | human) | SLRPEDTAVYYCASKLEYTSNDYDSWGQGTLVTVSS |
| 49 | MSLN DNA | acgcgtgtagtcttatgcaatactcttgtagtcttgcaacatggtaacgatgagttagcaacatgccttac |
| | Seq. | aaggagagaaaaagcaccgtgcatgccgattggtggaagtaaggtggtacgatcgtgccttattagg |
| | | aaggcaacagacgggtctgacatggattggacgaaccactgaattgccgcattgcagagatattgtat |
| | | ttaagtgcctagctcgatacaataaacgggtctctctggttagaccagatctgagcctgggagctctctg |
| | | gctaactagggaacccactgcttaagcctcaataaagcttgccttgagtgcttcaagtagtgtgtgccc |
| | | gtctgttgtgtgactctggtaactagagatccctcagacccttttagtcagtgtggaaaatctctagcagt |
| | | ggcgcccgaacagggacctgaaagcgaaagggaaaccagagctctctcgacgcaggactcggctt |
| | | gctgaagcgcgcacggcaagaggcgaggggcggcgactggtgagtacgccaaaaattttgactag |
| | | cggaggctagaaggagagagatgggtgcgagagcgtcagtattaagcgggggagaattagatcgc |
| | | gatgggaaaaaattcggttaaggccagggggaaagaaaaaatataaattaaaacatatagtatgggc |
| | | aagcagggagctagaacgattcgcagttaatcctggcctgttagaaacatcagaaggctgtagacaa |
| | | atactgggacagctacaaccatcccttcagacaggatcagaagaacttagatcattatataatacagta |
| | | gcaaccctctattgtgtgcatcaaaggatagagataaaagacaccaaggaagctttagacaagataga |
| | | ggaagagcaaaacaaaagtaagaccaccgcacagcaagcggccactgatcttcagacctggagga |
| | | ggagatatgagggacaattggagaagtgaattatataaatataaagtagtaaaaattgaaccattagga |
| | | gtagcacccaccaaggcaaagagaagagtggtgcagagagaaaaaagagcagtgggaataggag |
| | | ctttgttccttgggttcttgggagcagcaggaagcactatgggcgcagcctcaatgacgctgacggta |
| | | caggccagacaattattgtctggtatagtgcagcagcagaacaatttgctgagggctattgaggcgca |
| | | acagcatctgttgcaactcacagtctggggcatcaagcagctccaggcaagaatcctggctgtggaa |
| | | agatacctaaaggatcaacagctcctggggatttggggttgctctggaaaactcatttgcaccactgct |
| | | gtgccttggaatgctagttggagtaataaatctctggaacagattggaatcacacgacctggatggagt |
| | | gggacagagaaattaacaattacacaagcttaatacactccttaattgaagaatcgcaaaaccagcaa |
| | | gaaaagaatgaacaagaattattggaattagataaatgggcaagtttgtggaattggtttaacataacaa |
| | | attggctgtggtatataaaattattcataatgatagtaggaggcttggtaggtttaagaatagtttttgctgt |
| | | actttctatagtgaatagagttaggcagggatattcaccattatcgtttcagacccacctcccaaccccg |
| | | aggggacccgacaggcccgaaggaatagaagaagaaggtggagagagagacagagacagatcc |
| | | attcgattagtgaacggatctcgacggtatcggttaacttttaaaagaaaaggggggattggggggtac |
| | | agtgcaggggaaagaatagtagacataatagcaacagacatacaaactaaagaattacaaaaacaaa |
| | | ttacaaaattcaaaattttatcgatactagtattatgcccagtacatgaccttatgggactttcctacttggc |
| | | agtacatctacgtattagtcatcgctattaccatggtgatgcggttttggcagtacatcaatgggcgtgga |
| | | tagcggtttgactcacggggatttccaagtctccaccccattgacgtcaatggga |
| | | gtttgttttggcaccaaaatcaacgggactttccaaaatgtcgtaacaactccgccccattgacgcaaat |
| | | gggcggtaggcgtgtacggtgggaggtttatataagcagagctcgtttagtgaaccgtcagatcgcct |
| | | ggagacgccatccacgctgttttgacctccatagaagattctagagccgccaccatgcttctcctggtg |
| | | acaagccttctgctctgtgagttaccacacccagcattcctcctgatcccagacattcagcaggtccag |
| | | ctccagcagtctggccctgaactcgaaaaacctggcgctagcgtgaaaatttcctgtaaagcctccgg |
| | | ctactctatactggctacacaatgaattgggtgaaacagtctcacggcaaatccctcgaatggatcgga |
| | | ctcatcacaccctacaatggcgcctcttcctacaaccagaaattccggggcaaggcaacactcactgt |
| | | ggacaaatcatcctctaccgcctacatggatctgctctccctcacatctgaggactccgctgtctactttt |
| | | gtgcccgaggaggatacgacggacgaggattcgattactggggacagggaacaactgtgaccgtgt |
| | | ctagtggcggcggagggagtggaggcggaggatcttctggcgggggatccgatattgaactcacac |
| | | agtctcccgctatcatgtctgcttctcccggcgagaaagtgactatgacttgctctgcttcctcttctgtgt |
| | | cctacatgcactggtaccagcagaaatctggcacatcccctaaacggtggatctacgatactagcaaa |
| | | ctggcatccggcgtgcctgggcgattctctggctctggctctggcaactcttactctctcacaatctcatc |
| | | tgtcgaggctgaggacgatgccacatactactgtcagcagtggtctaaacacccactcacattcggcg |
| | | ctggcactaaactggaaataaaagcggccgcaggtggcggcggttctggtggcggcggttctggtg |
| | | gcggcggttctctcgaggatggtaatgaagaaatgggtggtattacacagacaccatataaagtctcc |
| | | atctctggaaccacagtaatattgacatgccctcagtatcctggatctgaaatactatggcaacacaatg |
| | | ataaaaacataggcggtgatgaggatgataaaaacataggcagtgatgaggatcacctgtcactgaa |
| | | ggaattttcagaattggagcaaagtggttattatgtctgctaccccagaggaagcaaaccagaagatgc |
| | | gaacttttatctctacctgagggcaagagtgtgtgagaactgcatggagatggatgtgatgtcggtggc |
| | | cacaattgtcatagtggacatctgcatcactgggggcttgctgctgctggtttactactggagcaagaat |
| | | agaaaggccaaggccaagcctgtgacacgaggagcgggtgctggcggcaggcaaaggggacaa |
| | | aacaaggagaggccaccacctgttcccaacccagactatgagcccatccggaaaggccagcggga |
| | | cctgtattctggcctgaatcagagacgcatctgataagaattcgatccgcggccgcgaaggatctgcg |
| | | atcgctccggtgcccgtcagtgggcagagcgcacatcgcccacagtccccgagaagttgggggga |
| | | ggggtcggcaattgaacgggtgcctagagaaggtggcgcggggtaaactgggaaagtgatgtcgtg |
| | | tactggctccgcctttttcccgagggtgggggagaaccgtatataagtgcagtagtcgccgtgaacgtt |
| | | ctttttcgcaacgggtttgccgccagaacacagctgaagcttcgaggggctcgcatctctccttcacgc |
| | | gcccgccgccctacctgaggccgccatccacgccggttgagtcgcgttctgccgcctcccgcctgtg |
| | | gtgcctcctgaactgcgtccgccgtctaggtaagtttaaagctcaggtcgagaccgggcctttgtccgg |
| | | cgctccatggagcctacctagactcagccggctctccacgctttgcctgaccctgcttgctcaactcta |
| | | cgtctttgtttcgttttctgttctgcgccgttacagatccaagctgtgaccggcgcctacgctagatgacc |
| | | gagtacaagcccacggtgcgcctcgccacccgcgacgacgtccccagggccgtacgcaccctcgc |
| | | cgccgcgttcgccgactaccccgccacgcgccacaccgtcgatccggaccgccacatcgagcggg |
| | | tcaccgagctgcaagaactatcctcacgcgcgtcgggctcgacatcggcaaggtgtgggtcgcgga |
| | | cgacggcgccgcggtggcggtctggaccacgccggagagcgtcgaagcgggggcggtgttcgcc |
| | | gagatcggcccgcgcatggccgagttgagcggttcccggctggccgcgcagcaacagatggaagg |
| | | cctcctggcgccgcaccggcccaaggagcccgcgtggttcctggccaccgtcggcgtctcgcccga |
| | | ccaccagggcaagggtctgggcagcgccgtcgtgctccccggagtggaggcggccgagcgcgcc |
| | | ggggtgcccgccttcctggagacctccgcgccccgcaacctccccttctacgagcggctcggcttca |
| | | ccgtcaccgccgacgtcgaggtgcccgaaggaccgcgcacctggtgcatgacccgcaagcccggt |
| | | gcctgagtcgacaatcaacctctggattacaaaatttgtgaaagattgactggtattcttaactatgttgct |
| | | ccttttacgctatgtggatacgctgctttaatgcctttgtatcatgctattgcttcccgtatggctttcatttt |
| | | ctcctccttgtataaatcctggttgctgtctctttatgaggagttgtggcccgttgtcaggcaacgtggcgtg |
| | | gtgtgcactgtgtttgctgacgcaacccccactggttggggcattgccaccacctgtcagctcctttccg |
| | | ggactttcgctttccccctccctattgccacggcggaactcatcgccgcctgccttgcccgctgctgga |
| | | caggggctcggctgttgggcactgacaattccgtggtgttgtcggggaaatcatcgtcctttccttggct |
| | | gctcgcctgtgttgccacctggattctgcgcgggacgtccttctgctacgtcccttcggccctcaatcca |
| | | gcggaccttccttcccgcggcctgctgccggctctttggcctcttccgcgtcttcgccttcgccctcag |
| | | acgagtcggatctccattgggccgcctccccgcctggtacctttaagaccaatgacttacaaggcag |
| | | ctgtagatcttagccactttttaaaagaaaaggggggactggaagggctaattcactcccaacgaaaat |
| | | aagatctgctttttgatgtactgggtctctctggttagaccagatctgagcctgggagctctctggctaa |
| | | ctagggaacccactgcttaagcctcaataaagcttgccttgagtgcttcaagtagtgtgtgcccgtctgtt |
| | | gtgtgactctggtaactagagatccctcagacccttttagtcagtgtggaaaatctctagcagtagtagtt |
| | | catgtcatcttattattcagtatttataacttgcaaagaaatgaatatcagagagtgagaggaacttgtttat |
| | | tgcagatataatggttacaaataaagcaatagcatcacaaatttcacaaataaagcattttatcactgca |
| | | ttctagttgtggtttgtccaaactcatcaatgtatcttatcatgtctggctctagctatcccgcccctaactcc |
| | | gcccagttccgcccattctccgccccatggctgactaattttttttatttatgcagaggccgaggccgcct |
| | | cggcctctgagctattccagaagtagtgaggaggcttttttggaggcctagacttttgcagagacggcc |
| | | caaattcgtaatcatggtcatagctgtttcctgtgtgaaattgttatccgctcacaattccacacaacatac |
| | | gagccggaagcataaagtgtaaagcctggggtgcctaatgagtgagctaactcacattaattgcgttg |
| | | cgctcactgcccgctttccagtcgggaaacctgtcgtgccagctgcattaatgaatcggccaacgcgc |
| | | ggggagaggcggtttgcgtattgggcgctcttccgcttcctcgctcactgactcgctgcgctcggtcgt |
| | | tcggctgcggcgagcggtatcagctcactcaaaggcggtaatacggttatccacagaatcaggggat |
| | | aacgcaggaaagaacatgtgagcaaaaggccagcaaaaggccaggaaccgtaaaaaggccgcgt |
| | | tgctggcgtttttccataggctccgcccccctgacgagcatcacaaaaatcgacgctcaagtcagagg |
| | | tggcgaaacccgacaggactataaagataccaggcgtttccccctggaagctccctcgtgcgctctcc |
| | | tgttccgaccctgccgcttaccggatacctgtccgcattctcccttcgggaagcgtggcgctttctcata |
| | | gctcacgctgtaggtatctcagttcggtgtaggtcgttcgctccaagctgggctgtgtgcacgaacccc |
| | | ccgttcagcccgaccgctgcgccttatccggtaactatcgtcttgagtccaacccggtaagacacgact |
| | | tatcgccactggcagcagccactggtaacaggattagcagagcgaggtatgtaggcggtgctacaga |
| | | gttcttgaagtggtggcctaactacggctacactagaaggacagtatttggtatctgcgctctgctgaag |
| | | ccagttaccttcggaaaaagagttggtagctcttgatccggcaaacaaaccaccgctggtagcggtgg |
| | | tttttttgtttgcaagcagcagattacgcgcagaaaaaaaggatctcaagaagatcctttgatcttttctac |
| | | ggggtctgacgctcagtggaacgaaaactcacgttaagggattttggtcatgagattatcaaaaaggat |
| | | cttcacctagatcatttaaattaaaaatgaagttttaaatcaatctaaagtatatatgagtaaacttggtctg |
| | | acagttaccaatgcttaatcagtgaggcacctatctcagcgatctgtctatttcgttcatccatagttgcct |
| | | gactccccgtcgtgtagataactacgatacgggagggcttaccatctggccccagtgctgcaatgata |
| | | ccgcgagacccacgctcaccggctccagatttatcagcaataaaccagccagccggaagggccga |
| | | gcgcagaagtggtcctgcaactttatccgcctccatccagtctattaattgttgccgggaagctagagta |
| | | agtagttcgccagttaatagtttgcgcaacgttgttgccattgctacaggcatcgtggtgtcacgctcgtc |
| | | gtttggtatggcttcattcagctccggttcccaacgatcaaggcgagttacatgatcccccatgttgtgca |
| | | aaaaagcggttagctccttcggtcctccgatcgttgtcagaagtaagttggccgcagtgttatcactcat |
| | | ggttatggcagcactgcataattctcttactgtcatgccatccgtaagatgcttttctgtgactggtgagta |
| | | ctcaaccaagtcattctgagaatagtgtatgcggcgaccgagttgctcttgcccggcgtcaatacggg |
| | | ataataccgcgccacatagcagaactttaaaagtgctcatcattggaaaacgttcttcggggcgaaaac |
| | | tctcaaggatcttaccgctgttgagatccagttcgatgtaacccactcgtgcacccaactgatcttcagc |
| | | atcttttactttcaccagcgtttctgggtgagcaaaaacaggaaggcaaaatgccgcaaaaaagggaa |
| | | taagggcgacacggaaatgagaatactcatactcttcattttcaatattattgaagcatttatcagggna |
| | | ttgtctcatgagcggatacatatttgaatgtatttagaaaaataaacaaataggggttccgcgcacatttc |
| | | cccgaaaagtgccacctgacgtctaagaaaccattattatcatgacattaacctataaaaataggcgtat |
| | | cacgaggccattcgtctcgcgcgtttcggtgatgacggtgaaaacctctgacacatgcagctcccgg |
| | | agacggtcacagcttgtctgtaagcggatgccgggagcagacaagcccgtcagggcgcgtcagcg |
| | | ggtgttggcgggtgtcggggctggcttaactatgcggcatcagagcagattgtactgagagtgcacca |
| | | tatgcggtgtgaaataccgcacagatgcgtaaggagaaaataccgcatcaggcgccattcgccattca |
| | | ggctgcgcaactgttgggaagggcgatcggtgcgggcctcttcgctattacgccagctggcgaaag |
| | | ggggatgtgctgcaaggcgattaagagggtaacgccagggtatcccagtcacgacgttgtaaaacg |
| | | acggccagtgccaagctg |
| 50 | MSLN amino | MALPTARPLLGSCGTPALGSLLFLLFSLGWVQPSRTLAGETGQE |
| | acid sequence: | AAPLDGVLANPPNISSLSPRQLLGFPCAEVSGLSTERVRELAVAL |
| | human | AQKNVKLSTEQLRCLAHRLSEPPEDLDALPLDLLLFLNPDAFSGP |
| | mesothelin | QACTRFFSRITKANVDLLPRGAPERQRLLPAALACWGVRGSLLS |
| | sequence | EADVRALGGLACDLPGRFVAESAEVLLPRLVSCPGPLDQDQQE |
| | (UniProt | AARAALQGGGPPYGPPSTWSVSTMDALRGLLPVLGQPIIRSIPQG |
| | Accession No. | IVAAWRQRSSRDPSWRQPERTILRPRFRREVEKTACPSGKKAREI |
| | Q13421) | DESLIFYKKWELEACVDAALLATQMDRVNAIPFTYEQLDVLKH |
| | | KLDELYPQGYPESVIQHLGYLFLKMSPEDIRKWNVTSLETLKAL |
| | | LEVNKGHEMSPQVATLIDRFVKGRGQLDKDTLDTLTAFYPGYL |
| | | CSLSPEELSSVPPSSIWAVRPQDLDTCDPRQLDVLYPKARLAFQN |
| | | MNGSEYFVKIQSFLGGAPTEDLKALSQQNVSMDLATFMKLRTD |
| | | AVLPLTVAEVQKLLGPHVEGLKAEERIHRPVRDWILRQRQDDLD |
| | | TLGLGLQGGIPNGYLVLDLSMQEALSGTPCLLGPGPVLTVLALL |
| | | LASTLA |
| 51 | p510_anti- | acgcgtgtagtcttatgcaatactcttgtagtcttgcaacatggtaacgatgagttagcaacatgccttac |
| | MSLN_SS1_ | aaggagagaaaaagcaccgtgcatgccgattggtggaagtaaggtggtacgatcgtgccttattagg |
| | CD3ϵ DNA | aaggcaacagacgggtctgacatggattggacgaaccactgaattgccgcattgcagagatattgtat |
| | | ttaagtgcctagctcgatacaataaacgggtctctctggttagaccagatctgagcctgggagctctctg |
| | | gctaactagggaacccactgcttaagcctcaataaagcttgccttgagtgcttcaagtagtgtgtgccc |
| | | gtctgttgtgtgactctggtaactagagatccctcagacccttttagtcagtgtggaaaatctctagcagt |
| | | ggcgcccgaacagggacctgaaagcgaaagggaaaccagagctctctcgacgcaggactcggctt |
| | | gctgaagcgcgcacggcaagaggcgaggggcggcgactggtgagtacgccaaaaattttgactag |
| | | cggaggctagaaggagagagatgggtgcgagagcgtcagtattaagcgggggagaattagatcgc |
| | | gatgggaaaaaattcggttaaggccagggggaaagaaaaaatataaattaaaacatatagtatgggc |
| | | aagcagggagctagaacgattcgcagttaatcctggcctgttagaaacatcagaaggctgtagacaa |
| | | atactgggacagctacaaccatcccttcagacaggatcagaagaacttagatcattatataatacagta |
| | | gcaaccctctattgtgtgcatcaaaggatagagataaaagacaccaaggaagctttagacaagataga |
| | | ggaagagcaaaacaaaagtaagaccaccgcacagcaagcggccactgatcttcagacctggagga |
| | | ggagatatgagggacaattggagaagtgaattatataaatataaagtagtaaaaattgaaccattagga |
| | | gtagcacccaccaaggcaaagagaagagtggtgcagagagaaaaaagagcagtgggaataggag |
| | | ctttgttccttgggttcttgggagcagcaggaagcactatgggcgcagcctcaatgacgctgacggta |
| | | caggccagacaattattgtctggtatagtgcagcagcagaacaatttgctgagggctattgaggcgca |
| | | acagcatctgttgcaactcacagtctggggcatcaagcagctccaggcaagaatcctggctgtggaa |
| | | agatacctaaaggatcaacagctcctggggatttggggttgctctggaaaactcatttgcaccactgct |
| | | gtgccttggaatgctagttggagtaataaatctctggaacagattggaatcacacgacctggatggagt |
| | | gggacagagaaattaacaattacacaagcttaatacactccttaattgaagaatcgcaaaaccagcaa |
| | | gaaaagaatgaacaagaattattggaattagataaatgggcaagtttgtggaattggtttaacataacaa |
| | | attggctgtggtatataaaattattcataatgatagtaggaggcttggtaggtttaagaatagtttttgctgt |
| | | actttctatagtgaatagagttaggcagggatattcaccattatcgtttcagacccacctcccaaccccg |
| | | aggggacccgacaggcccgaaggaatagaagaagaaggtggagagagagacagagacagatcc |
| | | attcgattagtgaacggatctcgacggtatcggttaacttttaaaagaaaaggggggattggggggtac |
| | | agtgcaggggaaagaatagtagacataatagcaacagacatacaaactaaagaattacaaaaacaaa |
| | | ttacaaaattcaaaattttatcgatactagtattatgcccagtacatgaccttatgggactttcctacttggc |
| | | agtacatctacgtattagtcatcgctattaccatggtgatgcggttttggcagtacatcaatgggcgtgga |
| | | tagcggtttgactcacggggatttccaagtctccaccccattgacgtcaatgggagtttgttttggcacc |
| | | aaaatcaacgggactttccaaaatgtcgtaacaactccgccccattgacgcaaatgggcggtaggcgt |
| | | gtacggtgggaggthatataagcagagacgtttagtgaaccgtcagatcgcctggagacgccatcc |
| | | acgctgttttgacctccatagaagattctagagccgccaccatgatctcctggtgacaagccttctgctc |
| | | tgtgagttaccacacccagcattcctcctgatcccagacattcagcaggtccagctccagcagtctggc |
| | | cctgaactcgaaaaacctggcgctagcgtgaaaatttcctgtaaagcctccggctactcttttactggct |
| | | acacaatgaattgggtgaaacagtctcacggcaaatccctcgaatggatcggactcatcacaccctac |
| | | aatggcgcctcttcctacaaccagaaattccggggcaaggcaacactcactgtggacaaatcatcctc |
| | | taccgcctacatggatctgctctccctcacatctgaggactccgctgtctacttttgtgcccgaggagga |
| | | tacgacggacgaggattcgattactggggacagggaacaactgtgaccgtgtctagtggcggcgga |
| | | gggagtggaggcggaggatcttctggcgggggatccgatattgaactcacacagtctcccgctatcat |
| | | gtctgcttctcccggcgagaaagtgactatgacttgctctgcttcctcttctgtgtcctacatgcactggta |
| | | ccagcagaaatctggcacatcccctaaacggtggatctacgatactagcaaactggcatccggcgtg |
| | | cctgggcgattctctggctctggctctggcaactcttactctctcacaatctcatctgtcgaggctgagga |
| | | cgatgccacatactactgtcagcagtggtctaaacacccactcacattcggcgctggcactaaactgg |
| | | aaataaaagcggccgcaggtggcggcggttctggtggcggcggttctggtggcggcggttctctcg |
| | | aggatggtaatgaagaaatgggtggtattacacagacaccatataaagtctccatctctggaaccaca |
| | | gtaatattgacatgccctcagtatcctggatctgaaatactatggcaacacaatgataaaaacataggcg |
| | | gtgatgaggatgataaaaacataggcagtgatgaggatcacctgtcactgaaggaattttcagaattgg |
| | | agcaaagtggttattatgtctgctaccccagaggaagcaaaccagaagatgcgaacttttatctctacct |
| | | gagggcaagagtgtgtgagaactgcatggagatggatgtgatgtcggtggccacaattgtcatagtg |
| | | gacatctgcatcactgggggcttgctgctgctggtttactactggagcaagaatagaaaggccaaggc |
| | | caagcctgtgacacgaggagcgggtgctggcggcaggcaaaggggacaaaacaaggagaggcc |
| | | accacctgttcccaacccagactatgagcccatccggaaaggccagcgggacctgtattctggcctg |
| | | aatcagagacgcatctgataagaattcgatccgcggccgcgaaggatctgcgatcgctccggtgccc |
| | | gtcagtgggcagagcgcacatcgcccacagtccccgagaagttggggggaggggtcggcaattga |
| | | acgggtgcctagagaaggtggcgcggggtaaactgggaaagtgatgtcgtgtactggctccgcctttt |
| | | tcccgagggtgggggagaaccgtatataagtgcagtagtcgccgtgaacgttattttcgcaacgggtt |
| | | tgccgccagaacacagctgaagattcgaggggctcgcatctctccttcacgcgcccgccgccctacc |
| | | tgaggccgccatccacgccggttgagtcgcgttctgccgcctcccgcctgtggtgcctcctgaactgc |
| | | gtccgccgtctaggtaagtttaaagctcaggtcgagaccgggcctttgtccggcgctcccttggagcct |
| | | acctagactcagccggctctccacgctttgcctgaccctgcttgctcaactctacgtctttgtttcgttttct |
| | | gttctgcgccgttacagatccaagctgtgaccggcgcctacgctagatgaccgagtacaagcccacg |
| | | gtgcgcctcgccacccgcgacgacgtccccagggccgtacgcaccctcgccgccgcgttcgccga |
| | | ctaccccgccacgcgccacaccgtcgatccggaccgccacatcgagcgggtcaccgagctgcaag |
| | | aactcttcctcacgcgcgtcgggctcgacatcggcaaggtgtgggtcgcggacgacggcgccgcg |
| | | gtggcggtctggaccacgccggagagcgtcgaagcgggggcggtgttcgccgagatcggcccgc |
| | | gcatggccgagttgagcggttcccggctggccgcgcagcaacagatggaaggcctcctggcgccg |
| | | caccggcccaaggagcccgcgtggttcctggccaccgtcggcgtctcgcccgaccaccagggcaa |
| | | gggtctgggcagcgccgtcgtgctccccggagtggaggcggccgagcgcgccggggtgcccgcc |
| | | ttcctggagacctccgcgccccgcaacctccccttctacgagcggctcggcttcaccgtcaccgccg |
| | | acgtcgaggtgcccgaaggaccgcgcacctggtgcatgacccgcaagcccggtgcctgagtcgac |
| | | aatcaacctctggattacaaaatagtgaaagattgactggtattcttaactatgttgctccttttacgctatg |
| | | tggatacgctgctttaatgcctttgtatcatgctattgcttcccgtatggctttcattttctcctccttgtata |
| | | aatcctggttgctgtctctttatgaggagttgtggcccgttgtcaggcaacgtggcgtggtgtgcactgtgtt |
| | | tgctgacgcaacccccactggttggggcattgccaccacctgtcagctcctttccgggactttcgctttc |
| | | cccctccctattgccacggcggaactcatcgccgcctgccttgcccgctgctggacaggggctcggc |
| | | tgttgggcactgacaattccgtggtgttgtcggggaaatcatcgtcctttccttggctgctcgcctgtgtt |
| | | gccacctggattctgcgcgggacgtccttctgctacgtcccttcggccctcaatccagcggaccttcctt |
| | | cccgcggcctgctgccggctctgcggcctcttccgcgtcttcgccttcgccctcagacgagtcggatc |
| | | tccctttgggccgcctccccgcctggtacctttaagaccaatgacttacaaggcagctgtagatcttagc |
| | | cactttttaaaagaaaaggggggactggaagggctaattcactcccaacgaaaataagatctgctttttg |
| | | cttgtactgggtctctctggttagaccagatctgagcctgggagctctctggctaactagggaacccact |
| | | gcttaagcctcaataaagcttgccttgagtgcttcaagtagtgtgtgcccgtctgttgtgtgactctggta |
| | | actagagatccctcagaccatttagtcagtgtggaaaatctctagcagtagtagttcatgtcatcttattat |
| | | tcagtatttataacttgcaaagaaatgaatatcagagagtgagaggaacttgtttattgcagcttataatg |
| | | gttacaaataaagcaatagcatcacaaatttcacaaataaagcatttttttcactgcattctagttgtggttt |
| | | gtccaaactcatcaatgtatcttatcatgtctggctctagctatcccgcccctaactccgcccagttccgc |
| | | ccattctccgccccatggctgactaattttttttatttatgcagaggccgaggccgcctcggcctctgagc |
| | | tattccagaagtagtgaggaggatttttggaggcctagacttttgcagagacggcccaaattcgtaatc |
| | | atggtcatagctgtttcctgtgtgaaattgttatccgctcacaattccacacaacatacgagccggaagc |
| | | ataaagtgtaaagcctggggtgcctaatgagtgagctaactcacattaattgcgttgcgctcactgccc |
| | | gattccagtcgggaaacctgtcgtgccagctgcattaatgaatcggccaacgcgcggggagaggc |
| | | ggtttgcgtattgggcgctcttccgcttcctcgctcactgactcgctgcgctcggtcgttcggctgcggc |
| | | gagcggtatcagctcactcaaaggcggtaatacggttatccacagaatcaggggataacgcaggaaa |
| | | gaacatgtgagcaaaaggccagcaaaaggccaggaaccgtaaaaaggccgcgttgctggcgttttt |
| | | ccataggctccgcccccctgacgagcatcacaaaaatcgacgctcaagtcagaggtggcgaaaccc |
| | | gacaggactataaagataccaggcgatccccctggaagctccctcgtgcgctctcctgttccgaccct |
| | | gccgcttaccggatacctgtccgcctttctcccttcgggaagcgtggcgctttctcatagctcacgctgt |
| | | aggtatctcagttcggtgtaggtcgttcgctccaagctgggctgtgtgcacgaaccccccgttcagccc |
| | | gaccgctgcgccttatccggtaactatcgtcttgagtccaacccggtaagacacgacttatcgccactg |
| | | gcagcagccactggtaacaggattagcagagcgaggtatgtaggcggtgctacagagttcttgaagt |
| | | ggtggcctaactacggctacactagaaggacagtatttggtatctgcgctctgctgaagccagttacctt |
| | | cggaaaaagagttggtagctcttgatccggcaaacaaaccaccgctggtagcggtggtttttttgtttgc |
| | | aagcagcagattacgcgcagaaaaaaaggatctcaagaagatcctttgatcttttctacggggtctgac |
| | | gctcagtggaacgaaaactcacgttaagggattttggtcatgagattatcaaaaaggatcttcacctaga |
| | | tccttttaaattaaaaatgaagttttaaatcaatctaaagtatatatgagtaaacttggtctgacagttacca |
| | | atgcttaatcagtgaggcacctatctcagcgatctgtctatttcgttcatccatagttgcctgactccccgt |
| | | cgtgtagataactacgatacgggagggcttaccatctggccccagtgctgcaatgataccgcgagac |
| | | ccacgctcaccggctccagatttatcagcaataaaccagccagccggaagggccgagcgcagaagt |
| | | ggtcctgcaactttatccgcctccatccagtctattaattgttgccgggaagctagagtaagtagttcgcc |
| | | agttaatagtttgcgcaacgttgttgccattgctacaggcatcgtggtgtcacgctcgtcgtttggtatgg |
| | | cttcattcagctccggttcccaacgatcaaggcgagttacatgatcccccatgttgtgcaaaaaagcgg |
| | | ttagctccttcggtcctccgatcgttgtcagaagtaagttggccgcagtgttatcactcatggttatggca |
| | | gcactgcataattctcttactgtcatgccatccgtaagatgcttttctgtgactggtgagtactcaaccaag |
| | | tcattctgagaatagtgtatgcggcgaccgagttgctcttgcccggcgtcaatacgggataataccgcg |
| | | ccacatagcagaactttaaaagtgctcatcattggaaaacgttcttcggggcgaaaactctcaaggatc |
| | | ttaccgctgttgagatccagttcgatgtaacccactcgtgcacccaactgatcttcagcatcttttactttc |
| | | accagcgtttctgggtgagcaaaaacaggaaggcaaaatgccgcaaaaaagggaataagggcgacacg |
| | | gaaatgttgaatactcatactcttcctttttcaatattattgaagcatttatcagggttattgtctcatgag |
| | | cggatacatatttgaatgtatttagaaaaataaacaaataggggttccgcgcacatttccccgaaaagtg |
| | | ccacctgacgtctaagaaaccattattatcatgacattaacctataaaaataggcgtatcacgaggccct |
| | | ttcgtctcgcgcgtttcggtgatgacggtgaaaacctctgacacatgcagctcccggagacggtcaca |
| | | gcttgtctgtaagcggatgccgggagcagacaagcccgtcagggcgcgtcagcgggtgttggcgg |
| | | gtgtcggggctggcttaactatgcggcatcagagcagattgtactgagagtgcaccatatgcggtgtg |
| | | aaataccgcacagatgcgtaaggagaaaataccgcatcaggcgccattcgccattcaggctgcgcaa |
| | | ctgttgggaagggcgatcggtgcgggcctcttcgctattacgccagctggcgaaagggggatgtgct |
| | | gcaaggcgattaagttgggtaacgccagggttttcccagtcacgacgttgtaaaacgacggccagtg |
| | | ccaagctg |
| 52 | p510_anti- | MLLLVTSLLLCELPFLPAFLLIPDIQQVQLQQSGPELEKPGASVKIS |
| | MSLN_SS1_ | CKASGYSFTGYTMNWVKQSHGKSLEWIGLITPYNGASSYNQKF |
| | CD3ϵ amino | RGKATLTVDKSSSTAYMDLLSLTSEDSAVYFCARGGYDGRGFD |
| | acid | YWGQGTTVTVSSGGGGSGGGGSSGGGSDIELTQSPAIMSASPGE |
| | | KVTMTCSASSSVSYMHWYQQKSGTSPKRWIYDTSKLASGVPGR |
| | | FSGSGSGNSYSLTISSVEAEDDATYYCQQWSKHPLTFGAGTKLEI |
| | | KAAAGGGGSGGGGSGGGGSLEDGNEEMGGITQTPYKVSISGTT |
| | | VILTCPQYPGSEILWQHNDKNIGGDEDDKNIGSDEDHLSLKEFSE |
| | | LEQSGYYVCYPRGSKPEDANFYLYLRARVCENCMEMDVMSVA |
| | | TIVIVDICITGGLLLLVYYWSKNRKAKAKPVTRGAGAGGRQRG |
| | | QNKERPPPVPNPDYEPIRKGQRDLYSGLNQRRI* |
| 53 | Anti-MSLN | DVVMTQTPLSLPVSLGDQASISCRSSQSLVHSNGNTYLHWYLQK |
| | Light Chain | PGQSPKLLIYKVSNRFSGVPDRFSGSGSGTDFTLKITRVEAEDLG |
| | amino acid | VFFCSQSTHVPFTFGSGTKLEIK |
| | (MHC1445LC.1) | |
| 54 | Anti-MSLN | gatgttgtgatgacccaaactccactctccctgcctgtcagtcttggagatcaagcctccatctcttgcag |
| | Light Chain | atctagtcagagccttgtacacagtaatggaaacacctatttacattggtacctgcagaagccaggcca |
| | DNA | gtctccaaagctcctgatctacaaagtttccaaccgattttctggggtcccagacaggttcagtggcagt |
| | (MHC1445LC.1) | ggatcagggactgatttcacactcaagatcaccagagtggaggctgaggatctgggagtttttttctgct |
| | | ctcaaagtacacatgttccattcacgttcggctcggggacaaagttggaaataaaa |
| 55 | Anti-MSLN | QVQLQQSGAELVRPGASVTLSCKASGYTFFDYEMHWVKQTPV |
| | Heavy Chain | HGLEWIGAIDPEIDGTAYNQKFKGKAILTADKSSSTAYMELRSL |
| | amino acid | TSEDSAVYYCTDYYGSSYWYFDVWGTGTTVTVSS |
| | (MHC1445HC.1) | |
| 56 | Anti-MSLN | caggttcaactgcagcagtctggggctgagctggtgaggcctggggcttcagtgacgctgtcctgca |
| | HeavyChain | aggcttcgggctacacattttttgactatgaaatgcactgggtgaagcagacacctgtgcatggcctgg |
| | DNA | aatggattggagctattgatcctgaaattgatggtactgcctacaatcagaagttcaagggcaaggcca |
| | (MHC1445HC.1) | tactgactgcagacaaatcctccagcacagcctacatggagctccgcagcctgacatctgaggactct |
| | | gccgtctattactgtacagattactacggtagtagctactggtacttcgatgtctggggcacagggacca |
| | | cggtcaccgtctcctc |
| 57 | Anti-MSLN | DVMMTQTPLSLPVSLGDQASISCRSSQSLVHSNGNTYLHWFLQK |
| | Light Chain | PGQSPKLLIYKVSNRFSGVPDRFSGSGSGTDFTLKISRVEAEDLG |
| | amino acid | VYFCSQTTHVPLTFGAGTKLELK |
| | (MHC1446LC.1) | |
| 58 | Anti-MSLN | gatgttatgatgacccaaactccactctccctgcctgtcagtcttggagatcaagcctccatctcttgcag |
| | Light Chain | atctagtcagagccttgtacacagtaatggaaacacctatttacattggttcctgcagaagccaggcca |
| | DNA | gtctccaaagctcctgatctacaaagtttccaaccgattttctggggtcccagacaggttcagtggcagt |
| | (MHC1446LC.1) | ggatcagggacagatttcacactcaagatcagcagagtggaggctgaggatctgggagtttatttctg |
| | | ctctcaaactacacatgttccgctcacgttcggtgctgggaccaagctggagctgaaa |
| 59 | Anti-MSLN | QVQLQQSGAELVRPGASVTLSCKASGYTFTDYEMHWVKQTPV |
| | Heavy Chain | HGLEWIGAIDPEIAGTAYNQKFKGKAILTADKSSSTAYMELRSL |
| | amino acid | TSEDSAVYYCSRYGGNYLYYFDYWGQGTTLTVSS |
| | (MHC1446HC.3) | |
| 60 | Anti-MSLN | caggttcaactgcagcagtctggggctgagctggtgaggcctggggcttcagtgacgctgtcctgca |
| | Heavy Chain | aggcttcgggctacacttttactgactatgaaatgcactgggtgaagcagacacctgtccatggcctgg |
| | DNA | aatggattggagctattgatcctgaaattgctggtactgcctacaatcagaagttcaagggcaaggcca |
| | (MHC1446HC.3) | tactgactgcagacaaatcctccagcacagcctacatggagctccgcagcctgacatctgaggactct |
| | | gccgtctattactgttcaagatacggtggtaactacctttactactttgactactggggccaaggcacca |
| | | ctctcacagtctcctca |
| 61 | Anti-MSLN | DVLMTQIPLSLPVSLGDQASISCRSSQNIVYSNGNTYLEWYLQKP |
| | Light Chain | GQSPKLLIYKVSNRFSGVPDRFSGSGSGTDFTLKISRVEAEDLGV |
| | amino acid | YYCFQGSHVPFTFGSGTKLEIK |
| | (MHC1447LC.5) | |
| 62 | Anti-MSLN | gatgttttgatgacccaaattccactctccctgcctgtcagtcttggagatcaagcctccatctcttgcag |
| | Light Chain | atctagtcagaacattgtgtatagtaatggaaacacctatttagagtggtacctgcagaaaccaggcca |
| | DNA | gtctccaaagctcctgatctacaaagtttccaaccgattttctggggtcccagacaggttcagtggcagt |
| | (MHC1447LC.5) | ggatcagggacagatttcacactcaagatcagcagagtggaggctgaggatctgggagtttattactg |
| | | ctttcaaggttcacatgttccattcacgttcggctcggggacaaagttggaaataaaa |
| 63 | Anti-MSLN | QVQLQQSGAELVRPGASVTLSCKASGYTFTDYEMHWVKQTPV |
| | Heavy Chain | HGLEWIGAIDPEIGGSAYNQKFKGRAILTADKSSSTAYMELRSLT |
| | amino acid | SEDSAVYYCTGYDGYFWFAYWGQGTLVTVSS |
| | (MHC1447HC.5) | |
| 64 | Anti-MSLN | caggttcaactgcagcagtccggggctgagctggtgaggcctggggcttcagtgacgctgtcctgca |
| | Heavy Chain | aggcttcgggctacacatttactgactatgaaatgcactgggtgaagcagacacctgtgcatggcctg |
| | DNA | gaatggattggagctattgatcctgaaattggtggttctgcctacaatcagaagttcaagggcagggcc |
| | (MHC1447HC.5) | atattgactgcagacaaatcctccagcacagcctacatggagctccgcagcctgacatctgaggactc |
| | | tgccgtctattattgtacgggctatgatggttacttttggtttgcttactggggccaagggactctggtcac |
| | | tgtctcttca |
| 65 | Anti-MSLN | ENVLTQSPAIMSASPGEKVTMTCSASSSVSYMHWYQQKSSTSPK |
| | Light Chain | LWIYDTSKLASGVPGRFSGSGSGNSYSLTISSMEAEDVATYYCF |
| | amino acid | QGSGYPLTFGSGTKLEIK |
| | (MHC1448LC.4) | |
| 66 | Anti-MSLN | gaaaatgttctcacccagtctccagcaatcatgtccgcatctccaggggaaaaggtcaccatgacctg |
| | Light Chain | cagtgctagctcaagtgtaagttacatgcactggtaccagcagaagtcaagcacctcccccaaactct |
| | DNA | ggatttatgacacatccaaactggcttctggagtcccaggtcgcttcagtggcagtgggtctggaaact |
| | (MHC1448LC.4) | cttactctctcacgatcagcagcatggaggctgaagatgttgccacttattactgattcaggggagtgg |
| | | gtacccactcacgttcggctcggggacaaagttggaaataaaa |
| 67 | Anti-MSLN | QVQLQQSGAELVRPGASVTLSCKASGYTFTDYEMHWVKQTPV |
| | Heavy Chain | HGLEWIGGIDPETGGTAYNQKFKGKALLTADKSSSTAYMELRSL |
| | amino acid | TSEDSAVYYCTSYYGSRVFWGTGTTVTVSS |
| | (MHC1448HC.3) | |
| 68 | Anti-MSLN | caggttcaactgcagcagtctggggctgagctggtgaggcctggggcttcagtgacgctgtcctgca |
| | Heavy Chain | aggcttcgggctacacatttactgactatgaaatgcactgggtgaaacagacacctgtgcatggcctg |
| | DNA | gaatggattggaggtattgatcctgaaactggtggtactgcctacaatcagaagttcaagggtaaggcc |
| | (MHC1448HC.3) | atactgactgcagacaaatcctccagcacagcctacatggagctccgcagcctgacatctgaggactc |
| | | tgccgtctattactgtacaagttactatggtagtagagtcttctggggcacagggaccacggtcaccgtc |
| | | tcctca |
| 69 | Anti-MSLN | QIVLSQSPAILSAFPGEKVTMTCRASSSVSYMFIWYQQKPGSSPK |
| | Light Chain | PWIYATSNLASGVPARFSGSGSGTSYSLTISSVEAEDAATYYCQQ |
| | amino acid | WSSNPPTLTFGAGTKLELK |
| | (MHC1449LC.3) | |
| 70 | Anti-MSLN | caaattgttctctcccagtctccagcaatcctgtctgcatttccaggggagaaggtcactatgacttgca |
| | Light Chain | gggccagctcaagtgtaagttacatgcactggtaccagcagaagccaggatcctcccccaaaccctg |
| | DNA | gatttatgccacatccaacctggcttctggagtccctgctcgcttcagtggcagtgggtctgggacctct |
| | (MHC1449LC.3) | tactctctcacaatcagcagtgtggaggctgaagatgctgccacttattactgccagcagtggagtagt |
| | | aacccacccacgctcacgttcggtgctgggaccaagctggagctgaaa |
| 71 | Anti-MSLN | QVQLQQSGAELARPGASVKLSCKASGYTFTSYGISWVKQRTGQ |
| | Heavy Chain | GLEWIGEIYPRSGNTYYNESFKGKVTLTADKSSGTAYMELRSLT |
| | amino acid | SEDSAVYFCARWGSYGSPPFYYGMDYWGQGTSVTVSS |
| | (MHC1449HC.3) | |
| 72 | Anti-MSLN | caggttcagctgcagcagtctggagctgagctggcgaggcctggggcttcagtgaagctgtcctgca |
| | Heavy Chain | aggcttctggctacaccttcacaagctatggtataagctgggtgaagcagaggactggacagggcctt |
| | DNA | gagtggattggagagatttatcctagaagtggtaatacttactacaatgagagcttcaagggcaaggtc |
| | (MHC1449HC.3) | acactgaccgcagacaaatcttccggcacagcgtacatggagctccgcagcctgacatctgaggact |
| | | ctgcggtctatttctgtgcaagatggggctcctacggtagtccccccttttactatggtatggactactgg |
| | | ggtcaaggaacctcagtcaccgtctcctca |
| 73 | Anti-MSLN | DVLMTQTPLSLPVSLGNQASISCRSSQSIVHSSGSTYLEWYLQKP |
| | Light Chain | GQSPKLLIYKVSNRFSGVPDRFSGSGSGTDFTLKISRVEAEDLGV |
| | amino acid | YYCFQGSHVPYTFGGGTKLEIK |
| | (MHC1450LC.3) | |
| 74 | Anti-MSLN | gatgttttgatgacccaaactccactctccctgcctgtcagtcttggaaatcaagcctccatctcttgcag |
| | Light Chain | atctagtcagagcattgtacatagtagtggaagcacctatttagaatggtacctgcagaaaccaggcca |
| | DNA | gtctccaaagctcctgatctacaaagtttccaaccgattttctggggtcccagacaggttcagtggcagt |
| | (MHC1450LC.3) | ggatcagggacagatttcacactcaagatcagcagagtggaggctgaggatctgggagatattactg |
| | | ctttcaaggctcacatgttccatacacgttcggaggggggaccaagctggaaataaaa |
| 75 | Anti-MSLN | QVQLQQSGAELARPGTSVKVSCKASGYTFTSYGISWVKQRIGQ |
| | Heavy Chain | GLEWIGEIHPRSGNSYYNEKIRGKATLTADKSSSTAYMELRSLIS |
| | amino acid | EDSAVYFCARLITTVVANYYAMDYWGQGTSVTVSS |
| | (MHC1450HC.5) | |
| 76 | Anti-MSLN | caggttcagctgcagcagtctggagctgagctggcgaggcctgggacttcagtgaaggtgtcctgca |
| | Heavy Chain | aggcttctggctataccttcacaagttatggtataagctgggtgaagcagagaattggacagggccttg |
| | DNA | agtggattggagagattcatcctagaagtggtaatagttactataatgagaagatcaggggcaaggcc |
| | (MHC1450HC.5) | acactgactgcagacaaatcctccagcacagcgtacatggagctccgcagcctgatatctgaggact |
| | | ctgcggtctatttctgtgcaaggctgattactacggtagttgctaattactatgctatggactactggggtc |
| | | aaggaacctcagtcaccgtctcctca |
| 77 | Anti-MSLN | DIVMSQSPSSLAVSAGEKVTMSCKSSQSLLNSRTRKNYLAWYQ |
| | Light Chain | QKPGQSPKLLIYWASTRESGVPDRFTGSGSGTDFTLTISSVQAED |
| | amino acid | LAVYYCKQSYNLVTFGAGTKLELK |
| | (MHC1451LC.1) | |
| 78 | Anti-MSLN | gacattgtgatgtcacagtctccatcctccctggctgtgtcagcaggagagaaggtcactatgagctgc |
| | Light Chain | aaatccagtcagagtctgctcaacagtagaacccgaaagaactacttggcttggtaccagcagaaacc |
| | DNA | agggcagtctcctaaactgctgatctactgggcatccactagggaatctggggtccctgatcgcttcac |
| | (MHC1451LC.1) | aggcagtggatctgggacagatttcactctcaccatcagcagtgtgcaggctgaagacctggcagttt |
| | | attactgcaaacaatcttataatctggtcacgttcggtgctgggaccaagctggagctgaaa |
| 79 | Anti-MSLN | QVQLQQSGAELVRPGASVTLSCKASGYTFFDYEMHWVKQTPV |
| | Heavy Chain | HGLEWIGAIDPEIDGTAYNQKFKGKAILTADKSSSTAYMELRSL |
| | amino acid | TSEDSAVYYCTDYYGSSYWYFDVWGTGTTVTVSS |
| | (MHC1451HC.2) | |
| 80 | Anti-MSLN | caggttcaactgcagcagtctggggctgagctggtgaggcctggggcttcagtgacgctgtcctgca |
| | Heavy Chain | aggcttcgggctacacattttttgactatgaaatgcactgggtgaagcagacacctgtgcatggcctgg |
| | DNA | aatggattggagctattgatcctgaaattgatggtactgcctacaatcagaagttcaagggcaaggcca |
| | (MHC1451HC.2) | tactgactgcagacaaatcctccagcacagcctacatggagctccgcagcctgacatctgaggactct |
| | | gccgtctattactgtacagattactacggtagtagctactggtacttcgatgtctggggcacagggacca |
| | | cggtcaccgtctcctc |
| 81 | Anti-MSLN | QIVLTQSPAIMSASPGEKVTISCSASSSVSYMYWYQQKPGSSPKP |
| | Light Chain | WIYRTSNLASGVPARFSGSGSGTSYSLTISSMEAEDAATYYCQQ |
| | amino acid | YHSYPLTFGAGTKLELK |
| | (MHC1452LC.1) | |
| 82 | Anti-MSLN | caaattgttctcacccagtctccagcaatcatgtctgcatctccaggggagaaggtcaccatatcctgca |
| | Light Chain | gtgccagctcaagtgtaagttacatgtactggtaccagcagaagccaggatcctcccccaaaccctgg |
| | DNA | atttatcgcacatccaacctggcttctggagtccctgctcgcttcagtggcagtgggtctgggacctctt |
| | (MHC1452LC.1) | actctctcacaatcagcagcatggaggctgaagatgctgccacttattactgccagcagtatcatagtta |
| | | cccactcacgttcggtgctgggaccaagctggagctgaaa |
| 83 | Anti-MSLN | QIVLTQSPAIMSASPGERVTMTCSASSSVSSSYLYWYQQKSGSSP |
| | Light Chain | KLWIYSISNLASGVPARFSGSGSGTSYSLTINSMEAEDAATYYCQ |
| | amino acid | QWSSNPQLTFGAGTKLELK |
| | (MHC1452LC.6) | |
| 84 | Anti-MSLN | caaattgttctcacccagtctccagcaatcatgtctgcatctcctggggaacgggtcaccatgacctgc |
| | Light Chain | agtgccagctcaagtgtaagttccagctacttgtactggtaccagcagaagtcaggatcctccccaaaa |
| | DNA | ctctggatttatagcatatccaacctggcttctggagtcccagctcgcttcagtggcagtgggtctggga |
| | (MHC1452LC.6) | cctcttactctctcacaatcaacagcatggaggctgaagatgctgccacttattactgccagcagtgga |
| | | gtagtaacccacagctcacgttcggtgctgggaccaagctggagctgaaa |
| 85 | Anti-MSLN | QVQLKQSGAELVKPGASVKISCKASGYTFTDYYINWVKQRPGQ |
| | Heavy Chain | GLEWIGKIGPGSGSTYYNEKFKGKATLTADKSSSTAYMQLSSLT |
| | amino acid | SEDSAVYFCARTGYYVGYYAMDYWGQGTSVTVSS |
| | (MHC1452HC.2) | |
| 86 | Anti-MSLN | caggtccagctgaagcagtctggagctgagctggtgaagcctggggcttcagtgaagatatcctgca |
| | Heavy Chain | aggcttctggctacaccttcactgactactatataaactgggtgaagcagaggcctggacagggcctt |
| | DNA | gagtggattggaaagattggtcctggaagtggtagtacttactacaatgagaagttcaagggcaaggc |
| | (MHC1452HC.2) | cacactgactgcagacaaatcctccagcacagcctacatgcagctcagcagcctgacatctgaggac |
| | | tctgcagtctatttctgtgcaagaactggttactacgttggttactatgctatggactactggggtcaagg |
| | | aacctcagtcaccgtctcctca |
| 87 | Anti-MSLN | QVQLQQSGAELARPGASVKLSCKASGYTFTIYGISWVKQRTGQ |
| | Heavy Chain | GLEWIGEIYPRSDNTYYNEKFKGKATLTADKSSSTAYMELRSLT |
| | amino acid | SEDSAVYFCARWYSFYAMDYWGQGTSVTVSS |
| | (MHC1452HC.4) | |
| 88 | Anti-MSLN | caggttcagctgcagcagtctggagctgagctggcgaggcctggggcttcagtgaagctgtcctgca |
| | Heavy Chain | aggcttctggctacaccttcacaatctatggtataagctgggtgaaacagagaactggacagggcctt |
| | DNA | gagtggattggagagatttatcctagaagtgataatacttactacaatgagaagttcaagggcaaggcc |
| | (MHC1452HC.4) | acactgactgcagacaaatcctccagcacagcgtacatggagctccgcagcctgacatctgaggact |
| | | ctgcggtctatttctgtgcaagatggtactcgttctatgctatggactactggggtcaaggaacctcagtc |
| | | accgtctcctca |
| 89 | Single domain | EVQLVESGGGLVQPGGSLRLSCAASGGDWSANFMYWYRQAPG |
| | anti-MSLN | KQRELVARISGRGVVDYVESVKGRFTISRDNSKNTLYLQMNSLR |
| | binder 1 | AEDTAVYYCAVASYWGQGTLVTVSS |
| | (SD1) | |
| 90 | Single domain | EVQLVESGGGLVQPGGSLRLSCAASGSTSSINTMYWYRQAPG |
| | anti-MSLN | KERELVAFISSGGSTNVRDSVKGRFTISRDNSKNTLYLQMNSLR |
| | binder 4 | AEDTAVYYCNTYIPYGGTLHDFWGQGTLVTVSS |
| | (SD4) | |
| 91 | Single domain | QVQLVESGGGVVQAGGSLRLSCAASGSTFSIRAMRWYRQAPG |
| | anti-MSLN | TERDLVAVIYGSSTYYADAVKGRFTISRDNSKNTLYLQMNSLRA |
| | binder 6 | EDTAVYYCNADTIGTARDYWGQGTLVTVSS |
| | (SD6) | |
| 92 | MUC16 | NFSPLARRVDRVAIYEEFLRMTRNGTQLQNFTLDRSSVLVDGYS |
| | immunization | PNRNEPLTGNSDLP |
| | peptide | |
| 93 | Modified | NFSPLARRVDRVAIYEEFLRMTRNGTQLQNFTLDRSSVLVDGYS |
| | MUC16 | PNRNEPLTGNSDLPC |
| | immunization | |
| | peptide | |
| 94 | Primer A6E | GATGTGCAGCTGCAGGAGTCTGGRGGAGG |
| 95 | Primer PMCF | CTAGTGCGGCCGCTGAGGAGACGGTGACCTGGGT |
| 96 | Universal | TCACACAGGAAACAGCTATGAC |
| | reverse primer | |
| 97 | Universal | CGCCAGGGTTTTCCCAGTCACGAC |
| | forward | |
| | primer | |
| 98 | RNA | AAUAAA |
| | polymerase | |
| | cleavage site | |
| 99 | MP057 primer | TTATGCTTCCGGCTCGTATG |

### Endnotes

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A composition comprising
(I) a first recombinant nucleic acid sequence encoding a first T cell receptor (TCR) fusion protein (TFP) comprising
(a) a TCR subunit comprising
(i) at least a portion of a TCR extracellular domain,
(ii) a transmembrane domain, and
(iii) a TCR intracellular domain comprising a stimulatory domain from an intracellular signaling domain derived only from a TCR subunit selected from the group consisting of a TCR alpha chain, a TCR beta chain, a TCR gamma chain, a TCR delta chain, a CD3 gamma chain, a CD3 delta chain and a CD3 epsilon chain; and
(b) a murine, human, or humanized antibody domain comprising an anti-MUC16 binding domain,
wherein the TCR subunit and the anti-MUC16 binding domain are operatively linked,
wherein the first TFP functionally interacts with a TCR or incorporates into a TCR when expressed in the T cell; and
(II) a second recombinant nucleic acid sequence encoding a second TFP comprising
(a) a TCR subunit comprising
(i) at least a portion of a TCR extracellular domain,
(ii) a transmembrane domain, and
(iii) a TCR intracellular domain comprising a stimulatory domain from an intracellular signaling domain derived only from a TCR subunit selected from the group consisting of a TCR alpha chain, a TCR beta chain, a TCR gamma chain, a TCR delta chain, a CD3 gamma chain, a CD3 delta chain and a CD3 epsilon chain; and
(b) a murine, human or humanized antibody domain comprising an anti-mesothelin (MSLN) binding domain,
wherein the TCR subunit and the anti-MSLN binding domain are operatively linked,
wherein the second TFP functionally interacts with a TCR or incorporates into a TCR when expressed in a T cell.

2. A composition comprising
(I) a first recombinant nucleic acid sequence encoding a first T cell receptor (TCR) fusion protein (TFP) comprising
(a) a TCR subunit comprising
(i) at least a portion of a TCR extracellular domain,
(ii) a transmembrane domain, and
(iii) a TCR intracellular domain comprising a stimulatory domain from an intracellular signaling domain derived only from a TCR subunit selected from the group consisting of a TCR alpha chain, a TCR beta chain, a TCR gamma chain, a TCR delta chain, a CD3 gamma chain, a CD3 delta chain and a CD3 epsilon chain; and
(b) a first human or humanized antibody domain comprising an anti-MUC16 binding domain and a second human or humanized antibody domain comprising an anti-MSLN binding domain;
wherein the TCR subunit, the first antibody domain, and the second antibody domain are operatively linked, and wherein the first TFP functionally interacts with a TCR or incorporates into a TCR when expressed in a T cell.

3. A composition comprising a recombinant nucleic acid molecule encoding:
(a) a first T cell receptor (TCR) fusion protein (TFP) comprising a TCR subunit, a first human or humanized antibody domain comprising a first antigen binding domain that is an anti-MUC16 binding domain; and
(b) a second T cell receptor (TCR) fusion protein (TFP) comprising a TCR subunit, a second human or humanized antibody domain comprising a second antigen binding domain that is an anti-MSLN binding domain,
wherein the TCR subunit of the first TFP and the first antibody domain are operatively linked and the TCR subunit of the second TFP and the second antibody domain are operatively linked.

4. A composition comprising a recombinant nucleic acid molecule encoding:
(a) a first T cell receptor (TCR) fusion protein (TFP) comprising a TCR subunit, a first human or humanized antibody domain comprising a first antigen binding domain that is an anti-MUC16 binding domain and a second human or humanized antibody domain comprising a second antigen binding domain that is an anti-MSLN binding domain; and
wherein the TCR subunit of the first TFP, the first antibody domain and the second antibody domain are operatively linked.

5. The composition of any one of claims 1-4, wherein the extracellular, transmembrane, and intracellular signaling domains of the TCR subunit of the first TFP are derived only from a TCR subunit selected from the group consisting of a TCR alpha chain, a TCR beta chain, a CD3 gamma chain, a CD3 delta chain and a CD3 epsilon chain.

6. The composition of any one of claims 1-5, wherein the extracellular, transmembrane, and intracellular signaling domains of the TCR subunit of the second TFP are derived only from a TCR subunit selected from the group consisting of a TCR alpha chain, a TCR beta chain, a TCR gamma chain, a TCR delta chain and a TCR epsilon chain.

7. The composition of claim 5 or 6, wherein the extracellular, transmembrane, and intracellular signaling domains of the TCR subunit of the first TFP are derived only from a TCR alpha chain.

8. The composition of claim 5 or 6, wherein the extracellular, transmembrane, and intracellular signaling domains of the TCR subunit of the first TFP are derived only from a TCR beta chain.

9. The composition of claim 5 or 6, wherein the extracellular, transmembrane, and intracellular signaling domains of the TCR subunit of the first TFP are derived only from a CD3 gamma chain.

10. The composition of claim 5 or 6, wherein the extracellular, transmembrane, and intracellular signaling domains of the TCR subunit of the first TFP are derived only from a CD3 delta chain.

11. The composition of claim 5 or 6, wherein the extracellular, transmembrane, and intracellular signaling domains of the TCR subunit of the first TFP are derived only from a CD3 epsilon chain.

12. The composition of any one of claims 5-11, wherein the extracellular, transmembrane, and intracellular signaling domains of the TCR subunit of the second TFP are derived only from a TCR alpha chain.

13. The composition of any one of claims 5-11, wherein the extracellular, transmembrane, and intracellular signaling domains of the TCR subunit of the second TFP are derived only from a TCR beta chain.

14. The composition of any one of claims 5-11, wherein the extracellular, transmembrane, and intracellular signaling domains of the TCR subunit of the second TFP are derived only from a CD3 gamma chain.

15. The composition of any one of claims 5-11, wherein the extracellular, transmembrane, and intracellular signaling domains of the TCR subunit of the second TFP are derived only from a CD3 delta chain.

16. The composition of any one of claims 5-11, wherein the extracellular, transmembrane, and intracellular signaling domains of the TCR subunit of the second TFP are derived only from a CD3 epsilon chain.

17. The composition of any one of claims 5-11, wherein the extracellular, transmembrane, and intracellular signaling domains of the TCR subunit of the second TFP are derived only from a TCR gamma chain.

18. The composition of any one of claims 5-11, wherein the extracellular, transmembrane, and intracellular signaling domains of the TCR subunit of the second TFP are derived only from a TCR delta chain.

19. The composition of any one of claims 3-16, wherein the first TFP, the second TFP, or both incorporate into a TCR or functionally interact with a TCR when expressed in a T cell.

20. The composition of any one of claims 3-19, wherein the first TFP, the second TFP, or both incorporate into a TCR or functionally interact with a TCR when expressed in a T cell.

21. The composition of any one of claims 1-20, wherein the encoded first antigen binding domain is connected to the TCR extracellular domain of the first TFP by a first linker sequence, the encoded second antigen binding domain is connected to the TCR extracellular domain of the second TFP by a second linker sequence, or both the first antigen binding domain is connected to the TCR extracellular domain of the first TFP by the first linker sequence and the encoded second antigen binding domain is connected to the TCR extracellular domain of the second TFP by the second linker sequence.

22. The composition of claim 21, wherein the first linker sequence and the second linker sequence comprise (G₄S)ₙ, wherein n=1 to 4.

23. The composition of any one of claims 1-22, wherein the TCR subunit of the first TFP, the TCR subunit of the second TFP, or both comprise a TCR extracellular domain.

24. The composition of any one of claims 1-23, wherein the TCR subunit of the first TFP, the TCR subunit of the second TFP, or both comprise a TCR transmembrane domain.

25. The composition of any one of claims 1-24, wherein the TCR subunit of the first TFP, the TCR subunit of the second TFP, or both comprise a TCR intracellular domain.

26. The composition of any one of claims 1-25, wherein the TCR subunit of the first TFP, the TCR subunit of the second TFP, or both comprise (i) a TCR extracellular domain, (ii) a TCR transmembrane domain, and (iii) a TCR intracellular domain, wherein at least two of (i), (ii), and (iii) are from the same TCR subunit.

27. The composition of any one of claims 1-26, wherein the TCR subunit of the first TFP, the TCR subunit of the second TFP, or both comprise a TCR intracellular domain comprising a stimulatory domain selected from an intracellular signaling domain of CD3 epsilon, CD3 gamma or CD3 delta, or an amino acid sequence having at least one modification thereto.

28. The composition of any one of claims 1-27, wherein the TCR subunit of the first TFP, the TCR subunit of the second TFP, or both comprise an intracellular domain comprising a stimulatory domain selected from a functional signaling domain of 4-1BB and/or a functional signaling domain of CD3 zeta, or an amino acid sequence having at least one modification thereto.

29. The composition of any one of claims 1-28, wherein the first human or humanized antibody domain, the second human or humanized antibody domain, or both comprise an antibody fragment.

30. The composition of any one of claims 1-29, wherein the first human or humanized antibody domain, the second human or humanized antibody domain, or both comprise a scFv or a V_{H} domain.

31. The composition of any one of claims 1-30, encoding (i) a light chain (LC) CDR1, LC CDR2 and LC CDR3 of a light chain binding domain amino acid sequence with 70-100% sequence identity to a light chain sequence of Table 2, and/or (ii) a heavy chain (HC) CDR1, HC CDR2 and HC CDR3 of a heavy chain sequence of Table 2.

32. The composition of any one of claims 1-31, encoding a light chain variable region, wherein the light chain variable region comprises an amino acid sequence having at least one but not more than 30 modifications of a light chain variable region amino acid sequence of Table 2, or a sequence with 95-99% identity to a light chain variable region amino acid sequence of Table 2.

33. The composition of any one of claims 1-32, encoding a heavy chain variable region, wherein the heavy chain variable region comprises an amino acid sequence having at least one but not more than 30 modifications of a heavy chain variable region amino acid sequence of Table 2, or a sequence with 95-99% identity to a heavy chain variable region amino acid sequence of Table 2.

34. The composition of any one of claims 1-33, wherein the encoded first TFP, the encoded second TFP, or both include an extracellular domain of a TCR subunit that comprises an extracellular domain or portion thereof of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications.

35. The composition of any one of claims 1-34, wherein the encoded first TFP and the encoded second TFP include a transmembrane domain that comprises a transmembrane domain of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications.

36. The composition of any one of claims 1-35, wherein the encoded first TFP and the encoded second TFP include a transmembrane domain that comprises a transmembrane domain of a protein selected from the group consisting of a TCR alpha chain, a TCR beta chain, a TCR zeta chain, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications.

37. The composition of any one of claims 1-36, further comprising a sequence encoding a costimulatory domain.

38. The composition of claim 37, wherein the costimulatory domain is a functional signaling domain obtained from a protein selected from the group consisting of OX40, CD2, CD27, CD28, CDS, ICAM-1, LFA-1 (CD11a/CD18), ICOS (CD278), and 4-1BB (CD137), and amino acid sequences thereof having at least one but not more than 20 modifications thereto.

39. The composition of any one of claims 1-38, further comprising a sequence encoding an intracellular signaling domain

40. The composition of any one of claims 1-39, further comprising a leader sequence.

41. The composition of any one of claims 1-40, further comprising a protease cleavage site.

42. The composition of any one of claims 1-41, wherein the at least one but not more than 20 modifications thereto comprise a modification of an amino acid that mediates cell signaling or a modification of an amino acid that is phosphorylated in response to a ligand binding to the first TFP, the second TFP, or both.

43. The composition of any one of claims 1-42, wherein the isolated nucleic acid molecule is an mRNA.

44. The composition of any one of claims 1-43, wherein the first TFP, the second TFP, or both include an immunoreceptor tyrosine-based activation motif (ITAM) of a TCR subunit that comprises an ITAM or portion thereof of a protein selected from the group consisting of CD3 zeta TCR subunit, CD3 epsilon TCR subunit, CD3 gamma TCR subunit, CD3 delta TCR subunit, TCR zeta chain, Fc epsilon receptor 1 chain, Fc epsilon receptor 2 chain, Fc gamma receptor 1 chain, Fc gamma receptor 2a chain, Fc gamma receptor 2b1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79a, CD79b, CD89, CD278, CD66d, functional fragments thereof, and amino acid sequences thereof having at least one but not more than 20 modifications thereto.

45. The composition of claim 44, wherein the ITAM replaces an ITAM of CD3 gamma, CD3 delta, or CD3 epsilon.

46. The composition of claim 44, wherein the ITAM is selected from the group consisting of CD3 zeta TCR subunit, CD3 epsilon TCR subunit, CD3 gamma TCR subunit, and CD3 delta TCR subunit and replaces a different ITAM selected from the group consisting of CD3 zeta TCR subunit, CD3 epsilon TCR subunit, CD3 gamma TCR subunit, and CD3 delta TCR subunit.

47. The isolated nucleic acid molecule of any one of claims 1-46, further comprising a leader sequence.

48. A composition comprising a polypeptide molecule encoded by the nucleic acid molecule of the composition of any one of claims 1-47.

49. The composition of claim 48, wherein the polypeptide comprises a first polypeptide encoded by a first nucleic acid molecule and a second polypeptide encoded by a second nucleic acid molecule.

50. A composition comprising a recombinant TFP molecule encoded by the nucleic acid molecule of the composition of any one of claims 1-47.

51. A composition comprising a vector comprising a nucleic acid molecule encoding the polypeptide or recombinant TFP molecule of any one of claims 48-50.

52. The composition of claim 51, wherein the vector comprises a) a first vector comprising a first nucleic acid molecule encoding the first TFP; and b) a second vector comprising a second nucleic acid molecule encoding the second TFP.

53. The composition of claim 51 or 52, wherein the vector is selected from the group consisting of a DNA, an RNA, a plasmid, a lentivirus vector, adenoviral vector, a Rous sarcoma viral (RSV) vector, or a retrovirus vector.

54. The composition of any one of claims 51-53, further comprising a promoter.

55. The composition of any one of claims 51-54, wherein the vector is an in vitro transcribed vector.

56. The composition of any one of claims 51-55, wherein the nucleic acid molecule in the vector further encodes a poly(A) tail.

57. The composition of any one of claims 51-56, wherein the nucleic acid molecule in the vector further encodes a 3′UTR.

58. The composition of any one of claims 51-57, wherein the nucleic acid molecule in the vector further encodes a protease cleavage site.

59. A composition comprising a cell comprising the composition of any one of claims 1-58.

60. The composition of claim 59, wherein the cell is a human T cell.

61. The composition of claim 60, wherein the T cell is a CD8+ or CD4+ T cell.

62. The composition of any one of claims 59-61, further comprising a nucleic acid encoding an inhibitory molecule that comprises a first polypeptide that comprises at least a portion of an inhibitory molecule, associated with a second polypeptide that comprises a positive signal from an intracellular signaling domain.

63. The composition of claim 62, wherein the inhibitory molecule comprises a first polypeptide that comprises at least a portion of PD1 and a second polypeptide comprising a costimulatory domain and primary signaling domain.

64. A vector comprising the recombinant nucleic acid sequence of any one of claims 1-63.

65. A vector comprising the first recombinant nucleic acid sequence of claim 1 or claim 2.

66. A vector comprising the second recombinant nucleic acid sequence of claim 1 or claim 2.

67. A cell comprising the composition of any one of claims 1-63 or the vector of any one of claims 64-66.

68. A cell comprising the vector of claim 65.

69. A cell comprising the vector of claim 66.

70. The cell any one of claims 67-69, wherein the cell is a human T cell.

71. The cell of claim 70, wherein the T cell is a CD8+ or CD4+ T cell.

72. The cell of any one of claims 67-71, further comprising a nucleic acid encoding an inhibitory molecule that comprises a first polypeptide that comprises at least a portion of an inhibitory molecule, associated with a second polypeptide that comprises a positive signal from an intracellular signaling domain.

73. The cell of claim 72, wherein the inhibitory molecule comprises a first polypeptide that comprises at least a portion of PD1 and a second polypeptide comprising a costimulatory domain and primary signaling domain.

74. A human CD8+ or CD4+ T cell comprising at least two TFP molecules, the TFP molecules comprising an anti-MUC16 binding domain, an anti-MSLN binding domain, a TCR extracellular domain, a transmembrane domain, and an intracellular domain, wherein the TFP molecule is capable of functionally interacting with an endogenous TCR complex and/or at least one endogenous TCR polypeptide in, at and/or on the surface of the human CD8+ or CD4+ T cell.

75. A protein complex comprising:
i) a first TFP molecule comprising an anti-MUC16 binding domain, a TCR extracellular domain, a transmembrane domain, and an intracellular domain;
ii) a second TFP molecule comprising an anti-MSLN binding domain, a TCR extracellular domain, a transmembrane domain, and an intracellular domain; and
iii) at least one endogenous TCR subunit or endogenous TCR complex.

76. A protein complex comprising:
i) a TFP molecule comprising an anti-MUC16 binding domain, a TCR extracellular domain, a transmembrane domain, and an intracellular domain; and
ii) at least one endogenous TCR subunit or endogenous TCR complex.

77. A protein complex comprising:
i) a TFP molecule comprising an anti-MSLN binding domain, a TCR extracellular domain, a transmembrane domain, and an intracellular domain; and
ii) at least one endogenous TCR subunit or endogenous TCR complex

78. The protein complex of any one of claims 75-77, wherein the TCR comprises an extracellular domain or portion thereof of a protein selected from the group consisting of TCR alpha chain, a TCR beta chain, a CD3 epsilon TCR subunit, a CD3 gamma TCR subunit, and a CD3 delta TCR subunit.

79. The protein complex of any one of claims 76-78, wherein the anti-MUC16 binding domain, the anti-MSLN binding domain, or both are connected to the TCR extracellular domain by a linker sequence.

80. The protein complex of claim 79, wherein the linker region comprises (G₄S)ₙ, wherein n=1 to 4.

81. A human CD8+ or CD4+ T cell comprising at least two different TFP proteins per the protein complex of any one of claims 75-79.

82. A human CD8+ or CD4+ T cell comprising at least two different TFP molecules encoded by the isolated nucleic acid molecule of any one of claims 1-63.

83. A population of human CD8+ or CD4+ T cells, wherein the T cells of the population individually or collectively comprise at least two TFP molecules, the TFP molecules comprising an anti-MUC16 binding domain or an anti-MSLN binding domain, or both an anti-MUC16 and an anti-MSLN binding domain, a TCR extracellular domain, a transmembrane domain, and an intracellular domain, wherein the TFP molecule is capable of functionally interacting with an endogenous TCR complex and/or at least one endogenous TCR polypeptide in, at and/or on the surface of the human CD8+ or CD4+ T cell.

84. A population of human CD8+ or CD4+ T cells, wherein the T cells of the population individually or collectively comprise at least two TFP molecules encoded by the recombinant nucleic acid molecule of any one of claims 1-63.

85. A pharmaceutical composition comprising an effective amount of the composition of any one of claims 1-63, the vector of any one of claims 64-66, the cell of any one of claims 67-69, or the protein complex of any one of claims 75-80, and a pharmaceutically acceptable excipient.

86. A pharmaceutical composition comprising an effective amount of the cell of claim 68, the cell of claim 69, and a pharmaceutically acceptable excipient.

87. A method of treating a mammal having a disease associated with expression of MSLN or MUC16 comprising administering to the mammal an effective amount of the composition one any one of claims 1-63.

88. The method of claim 87, wherein the disease associated with MUC16 or MSLN expression is selected from the group consisting of a proliferative disease, a cancer, a malignancy, myelodysplasia, a myelodysplastic syndrome, a preleukemia, a non-cancer related indication associated with expression of MUC16, a non-cancer related indication associated with expression of MSLN, breast cancer, prostate cancer, ovarian cancer, cervical cancer, skin cancer, pancreatic cancer, colorectal cancer, renal cancer, liver cancer, brain cancer, lymphoma, leukemia, lung cancer, esophageal cancer, gastric cancer and unresectable ovarian cancer with relapsed or refractory disease.

89. The method of claim 87, wherein the disease is a hematologic cancer selected from the group consisting of B-cell acute lymphoid leukemia (B-ALL), T cell acute lymphoid leukemia (T-ALL), acute lymphoblastic leukemia (ALL); chronic myelogenous leukemia (CML), chronic lymphocytic leukemia (CLL), B cell prolymphocytic leukemia, blastic plasmacytoid dendritic cell neoplasm, Burkitt's lymphoma, diffuse large B cell lymphoma, follicular lymphoma, hairy cell leukemia, small cell-follicular lymphoma, large cell-follicular lymphoma, malignant lymphoproliferative conditions, MALT lymphoma, mantle cell lymphoma, Marginal zone lymphoma, multiple myeloma, myelodysplasia, myelodysplastic syndrome, non-Hodgkin's lymphoma, plasmablastic lymphoma, plasmacytoid dendritic cell neoplasm, Waldenstrom macroglobulinemia, preleukemia, a disease associated with MUC16 or MSLN expression, and combinations thereof.

90. The method of claim 87, wherein the cells expressing a first TFP molecule and a second TFP molecule are administered in combination with an agent that increases the efficacy of a cell expressing the first TFP molecule and the second TFP molecule.

91. The method of any one of claims 87-90, wherein less cytokines are released in the mammal compared a mammal administered an effective amount of a T cell expressing:
(a) an anti-MSLN chimeric antigen receptor (CAR);
(b) an anti-MUC16 CAR;
(c) an anti-MSLN CAR and an anti-MUC16 CAR; or
(d) a combination thereof.

92. The method of any one of claims 87-91, wherein the cells expressing the first TFP molecule and a second TFP molecule are administered in combination with an agent that ameliorates one or more side effects associated with administration of a cell expressing the first TFP molecule and the second TFP molecule.

93. The method of any one of claims 87-92, wherein the cells expressing the first TFP molecule and a second TFP molecule are administered in combination with an agent that treats the disease associated with MSLN or MUC16.


SEQUENCE LISTING (117 sequences)

SEQ ID NO: 1 - protein, 17 aa, Artificial Sequence
  source: /note=“Description of Artificial Sequence Synthetic peptide”
GGGGSGGGGSGGGGSLE

SEQ ID NO: 2 - protein, 21 aa, Artificial Sequence
  source: /note=“Description of Artificial Sequence Synthetic peptide”
GGGSGGGGSGGGGSGGGGSLE

SEQ ID NO: 3 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 4 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 5 - protein, 172 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKS

SEQ ID NO: 6 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 7 - protein, 280 aa, Homo sapiens
MAGTWLLLLLALGCPALPTGVGGTPFPSLAPPIMLLVDGKQQMVVVCLVLDVAPPGLDSPIWFSAGNGSALDAFTYGPSPATDGTWTNLAHLSLPSEELASWEPLVCHTGPGAEGHSRSTQPMHLSGEASTARTCPQEPLRGTPGGALWLGVLRLLLFKLLLFDLLLTCSCLCDPAGPLPSPATTTRLRALGSHRLHPATETGGREATSSPRPQPRDRRWGDTPPGRKPGSPVWGEGSYLSSYPTCPAQAWCSRSALRAPSSSLGAFFAGDLPPPLQAGA

SEQ ID NO: 8 - protein, 142 aa, Homo sapiens
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 9 - protein, 139 aa, Homo sapiens
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAAKGAGTASKLTFGTGTRLQVTL

SEQ ID NO: 10 - protein, 177 aa, Homo sapiens
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 11 - protein, 133 aa, Homo sapiens
MGTSLLCWMALCLLGADHADTGVSQNPRHNITKRGQNVTFRCDPISEHNRLYWYRQTLGQGPEFLTYFQNEAQLEKSRLLSDRFSAERPKGSFSTLEIQRTEQGDSAMYLCASSLAGLNQPQHFGDGTRLSIL

SEQ ID NO: 12 - protein, 135 aa, Homo sapiens
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSFSTCSANYGYTFGSGTRLTVV

SEQ ID NO: 13 - DNA, 360 bp, Artificial Sequence
  source: /note=“Description of Artificial Sequence Synthetic polynucleotide”
caggtgcagctgcaggagtctgggggaggattggtgcaggctgggggctctctgagactctcctgtgcagcctctggacgcaccgtcagtagcttgttcatgggctggttccgccaagctccagggaaggagcgtgaacttgtagcagccattagccggtatagtctatatacatactatgcagactccgtgaagggccgattcaccatctccgcagacaacgccaagaacgcggtatatctgcaaatgaacagcctgaaacctgaggacacggccgtttattactgtgcatcaaagttggaatatacttctaatgactatgactcctggggccaggggacccaggtcaccgtctcctca

SEQ ID NO: 14 - protein, 120 aa, Artificial Sequence
  source: /note=“Description of Artificial Sequence Synthetic polypeptide”
QVQLQESGGGLVQAGGSLRLSCAASGRTVSSLFMGWFRQAPGKERELVAAISRYSLYTYYADSVKGRFTISADNAKNAVYLQMNSLKPEDTAVYYCASKLEYTSNDYDSWGQGTQVTVSS

SEQ ID NO: 15 - protein, 8 aa, Artificial Sequence
  source: /note=“Description of Artificial Sequence Synthetic peptide”
GRTVSSLF

SEQ ID NO: 16 - protein, 8 aa, Artificial Sequence
  source: /note=“Description of Artificial Sequence Synthetic peptide”
ISRYSLYT

SEQ ID NO: 17 - protein, 13 aa, Artificial Sequence
  source: /note=“Description of Artificial Sequence Synthetic peptide”
ASKLEYTSNDYDS

SEQ ID NO: 18 - DNA, 360 bp, Artificial Sequence
  source: /note=“Description of Artificial Sequence Synthetic polynucleotide”
caggtgcagctgcaggagtctgggggaggattggtgcaggctggggactctctgagactctcctgtgcagcctctggacgcgccgtcagtagcttgttcatgggctggttccgccgagctccagggaaggagcgtgaacttgtagcagccattagccggtatagtctatatacatactatgcagactccgtgaagggccgattcaccatctccgcagacaacgccaagaacgcggtatatctgcaaatgaacagcctaaaacctgaggacacggccgtttattactgtgcatcaaagttggaatatacttctaatgactatgactcctggggccaggggacccaggtcaccgtctcctca

SEQ ID NO: 19 - protein, 120 aa, Artificial Sequence
  source: /note=“Description of Artificial Sequence Synthetic polypeptide”
QVQLQESGGGLVQAGDSLRLSCAASGRAVSSLFMGWFRRAPGKERELVAAISRYSLYTYYADSVKGRFTISADNAKNAVYLQMNSLKPEDTAVYYCASKLEYTSNDYDSWGQGTQVTVSS

SEQ ID NO: 20 - protein, 8 aa, Artificial Sequence
  source: /note=“Description of Artificial Sequence Synthetic peptide”
GRAVSSLF

SEQ ID NO: 21 - protein, 8 aa, Artificial Sequence
  source: /note=“Description of Artificial Sequence Synthetic peptide”
ISRYSLYT

SEQ ID NO: 22 - protein, 13 aa, Artificial Sequence
  source: /note=“Description of Artificial Sequence Synthetic peptide”
ASKLEYTSNDYDS

SEQ ID NO: 23 - DNA, 360 bp, Artificial Sequence
  source: /note=“Description of Artificial Sequence Synthetic polynucleotide”
caggtgcagctgcaggagtctgggggaggattggtgcaggctggggactctctgagactctcctgtgcagcctctggacgcaccgtcagtagcttgttcatggggtggttccgccgagctccagggaaggagcgtgaacttgtagcagccattagccggtatagtctatatacatactatgcagactccgtgaagggccgattcaccatctccgcagacaacgccaagaacgcggtatatctgcaaatgaacagcctgaaacctgaggacacggccgtttattactgtgcatcaaagttggaatatacttctaatgactatgactcctggggccaggggacccaggtcaccgtctcctca

SEQ ID NO: 24 - protein, 120 aa, Artificial Sequence
  source: /note=“Description of Artificial Sequence Synthetic polypeptide”
QVQLQESGGGLVQAGDSLRLSCAASGRTVSSLFMGWFRRAPGKERELVAAISRYSLYTYYADSVKGRFTISADNAKNAVYLQMNSLKPEDTAVYYCASKLEYTSNDYDSWGQGTQVTVSS

SEQ ID NO: 25 - protein, 8 aa, Artificial Sequence
  source: /note=“Description of Artificial Sequence Synthetic peptide”
GRTVSSLF

SEQ ID NO: 26 - protein, 8 aa, Artificial Sequence
  source: /note=“Description of Artificial Sequence Synthetic peptide”
ISRYSLYT

SEQ ID NO: 27 - protein, 13 aa, Artificial Sequence
  source: /note=“Description of Artificial Sequence Synthetic peptide”
ASKLEYTSNDYDS

SEQ ID NO: 28 - DNA, 348 bp, Artificial Sequence
  source: /note=“Description of Artificial Sequence Synthetic polynucleotide”
caggtgcagctgcaggagtctgggggaggtttggtgcagcctggggattctatgagactctcctgtgcagccgagggggactctttggatggttatgtagtaggttggttccgccaggccccagggaaggagcgccagggggtctcaagtattagtggcgatggcagtatgcgatacgttgctgactccgtgaaggggcgattcaccatctcccgagacaacgccaagaacacggtgtatctgcaaatgatcgacctgaaacctgaggacacaggcgtttattactgtgcagcagacccacccacttgggactactggggtcaggggacccaggtcaccgtctcctca

SEQ ID NO: 29 - protein, 116 aa, Artificial Sequence
  source: /note=“Description of Artificial Sequence Synthetic polypeptide”
QVQLQESGGGLVQPGDSMRLSCAAEGDSLDGYVVGWFRQAPGKERQGVSSISGDGSMRYVADSVKGRFTISRDNAKNTVYLQMIDLKPEDTGVYYCAADPPTWDYWGQGTQVTVSS

SEQ ID NO: 30 - protein, 8 aa, Artificial Sequence
  source: /note=“Description of Artificial Sequence Synthetic peptide”
GDSLDGYV

SEQ ID NO: 31 - protein, 8 aa, Artificial Sequence
  source: /note=“Description of Artificial Sequence Synthetic peptide”
ISGDGSMR

SEQ ID NO: 32 - protein, 9 aa, Artificial Sequence
  source: /note=“Description of Artificial Sequence Synthetic peptide”
AADPPTWDY

SEQ ID NO: 33 - DNA, 360 bp, Artificial Sequence
  source: /note=“Description of Artificial Sequence Synthetic polynucleotide”
caggtgcagctgcaggagtctgggggaggcttggtgcagcctggggggtctctgagactctcctgtgcagcctctggacgcaccgtcagtagcttgttcatgggctggttccgccgagctccagggaaggagcgtgaacttgtagcagccattagccggtatagtctatatacatactatgcagactccgtgaagggccgattcaccatctccgcagacaacgccaagaacgcggtatatctgcaaatgaacagcctgaaacctgaggacacggccgtttattactgtgcatcaaagttggaatatacttctaatgactatgactcctggggccaggggacccaggtcaccgtctcctca

SEQ ID NO: 34 - protein, 120 aa, Artificial Sequence
  source: /note=“Description of Artificial Sequence Synthetic polypeptide”
QVQLQESGGGLVQPGGSLRLSCAASGRTVSSLFMGWFRRAPGKERELVAAISRYSLYTYYADSVKGRFTISADNAKNAVYLQMNSLKPEDTAVYYCASKLEYTSNDYDSWGQGTQVTVSS

SEQ ID NO: 35 - protein, 8 aa, Artificial Sequence
  source: /note=“Description of Artificial Sequence Synthetic peptide”
GRTVSSLF

SEQ ID NO: 36 - protein, 8 aa, Artificial Sequence
  source: /note=“Description of Artificial Sequence Synthetic peptide”
ISRYSLYT

SEQ ID NO: 37 - protein, 13 aa, Artificial Sequence
  source: /note=“Description of Artificial Sequence Synthetic peptide”
ASKLEYTSNDYDS

SEQ ID NO: 38 - DNA, 360 bp, Artificial Sequence
  source: /note=“Description of Artificial Sequence Synthetic polynucleotide”
caggtgcagctgcaggagtctgggggaggattggtgcaggctggggagtctctgagactctcctgtgcagcctctggacgcaccgtcagtagcttgttcatgggctggttccgccgagctccagggaaggagcgtgaacttgtagcagccattagccggtatagtctatatacatactatgcagactccgtgaagggccgattcaccatctccgcagacaacgccaagaacgcggtatatctgcaaatgaacagcctgaaacctgaggacacggccgtttattactgtgcatcaaagttggaatatacttctaatgactatgactcctggggccaggggacccaggtcaccgtctcctca

SEQ ID NO: 39 - protein, 120 aa, Artificial Sequence
  source: /note=“Description of Artificial Sequence Synthetic polypeptide”
QVQLQESGGGLVQAGESLRLSCAASGRTVSSLFMGWFRRAPGKERELVAAISRYSLYTYYADSVKGRFTISADNAKNAVYLQMNSLKPEDTAVYYCASKLEYTSNDYDSWGQGTQVTVSS

SEQ ID NO: 40 - protein, 8 aa, Artificial Sequence
  source: /note=“Description of Artificial Sequence Synthetic peptide”
GRTVSSLF

SEQ ID NO: 41 - protein, 8 aa, Artificial Sequence
  source: /note=“Description of Artificial Sequence Synthetic peptide”
ISRYSLYT

SEQ ID NO: 42 - protein, 13 aa, Artificial Sequence
  source: /note=“Description of Artificial Sequence Synthetic peptide”
ASKLEYTSNDYDS

SEQ ID NO: 43 - protein, 120 aa, Artificial Sequence
  source: /note=“Description of Artificial Sequence Synthetic polypeptide”
EVQLVESGGGLVQPGGSLRLSCAASGRAVSSLFMGWVRQAPGKGLEWVSAISRYSLYTYYADSVKGRFTISRDNAKNTLYLQMNSLRPEDTAVYYCASKLEYTSNDYDSWGQGTLVTVSS

SEQ ID NO: 44 - protein, 120 aa, Artificial Sequence
  source: /note=“Description of Artificial Sequence Synthetic polypeptide”
EVQLVESGGGLVQPGGSLRLSCAASGRAVSSLFMGWFRQAPGKGLEWVSAISRYSLYTYYADSVKGRFTISRDNAKNTLYLQMNSLRPEDTAVYYCASKLEYTSNDYDSWGQGTLVTVSS

SEQ ID NO: 45 - protein, 120 aa, Artificial Sequence
  source: /note=“Description of Artificial Sequence Synthetic polypeptide”
EVQLVESGGGLVQPGGSLRLSCAASGRAVSSLFMGWFRQAPGKGLELVSAISRYSLYTYYADSVKGRFTISRDNAKNTLYLQMNSLRPEDTAVYYCASKLEYTSNDYDSWGQGTLVTVSS

SEQ ID NO: 46 - protein, 120 aa, Artificial Sequence
  source: /note=“Description of Artificial Sequence Synthetic polypeptide”
EVQLVESGGGLVQPGGSLRLSCAASGRTVSSLFMGWVRQAPGKGLEWVSAISRYSLYTYYADSVKGRFTISRDNAKNTLYLQMNSLRPEDTAVYYCASKLEYTSNDYDSWGQGTLVTVSS

SEQ ID NO: 47 - protein, 120 aa, Artificial Sequence
  source: /note=“Description of Artificial Sequence Synthetic polypeptide”
EVQLVESGGGLVQPGGSLRLSCAASGRTVSSLFMGWFRQAPGKGLEWVSAISRYSLYTYYADSVKGRFTISRDNAKNTLYLQMNSLRPEDTAVYYCASKLEYTSNDYDSWGQGTLVTVSS

SEQ ID NO: 48 - protein, 120 aa, Artificial Sequence
  source: /note=“Description of Artificial Sequence Synthetic polypeptide”
EVQLVESGGGLVQPGGSLRLSCAASGRTVSSLFMGWFRQAPGKGLELVSAISRYSLYTYYADSVKGRFTISRDNAKNTLYLQMNSLRPEDTAVYYCASKLEYTSNDYDSWGQGTLVTVSS

SEQ ID NO: 49 - DNA, 8791 bp, Unknown
  source: /note=“Description of Unknown MSLN sequence”
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagagccgccaccatgcttctcctggtgacaagccttctgctctgtgagttaccacacccagcattcctcctgatcccagacattcagcaggtccagctccagcagtctggccctgaactcgaaaaacctggcgctagcgtgaaaatttcctgtaaagcctccggctactcttttactggctacacaatgaattgggtgaaacagtctcacggcaaatccctcgaatggatcggactcatcacaccctacaatggcgcctcttcctacaaccagaaattccggggcaaggcaacactcactgtggacaaatcatcctctaccgcctacatggatctgctctccctcacatctgaggactccgctgtctacttttgtgcccgaggaggatacgacggacgaggattcgattactggggacagggaacaactgtgaccgtgtctagtggcggcggagggagtggaggcggaggatcttctggcgggggatccgatattgaactcacacagtctcccgctatcatgtctgcttctcccggcgagaaagtgactatgacttgctctgcttcctcttctgtgtcctacatgcactggtaccagcagaaatctggcacatcccctaaacggtggatctacgatactagcaaactggcatccggcgtgcctgggcgattctctggctctggctctggcaactcttactctctcacaatctcatctgtcgaggctgaggacgatgccacatactactgtcagcagtggtctaaacacccactcacattcggcgctggcactaaactggaaataaaagcggccgcaggtggcggcggttctggtggcggcggttctggtggcggcggttctctcgaggatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatctgataagaattcgatccgcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 50 - protein, 622 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 51 - DNA, 8791 bp, Artificial Sequence; identical to SEQ ID NO: 49
  source: /note=“Description of Artificial Sequence Synthetic polynucleotide”

SEQ ID NO: 52 - protein, 470 aa, Artificial Sequence
  source: /note=“Description of Artificial Sequence Synthetic polypeptide”
MLLLVTSLLLCELPHPAFLLIPDIQQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSSGGGSDIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGAGTKLEIKAAAGGGGSGGGGSGGGGSLEDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 53 - protein, 112 aa, Artificial Sequence
  source: /note=“Description of Artificial Sequence Synthetic polypeptide”
DVVMTQTPLSLPVSLGDQASISCRSSQSLVHSNGNTYLHWYLQKPGQSPKLLIYKVSNRFSGVPDRFSGSGSGTDFTLKITRVEAEDLGVFFCSQSTHVPFTFGSGTKLEIK

SEQ ID NO: 54 - DNA, 336 bp, Artificial Sequence
  source: /note=“Description of Artificial Sequence Synthetic polynucleotide”
gatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggactgatttcacactcaagatcaccagagtggaggctgaggatctgggagtttttttctgctctcaaagtacacatgttccattcacgttcggctcggggacaaagttggaaataaaa

SEQ ID NO: 55 - protein, 120 aa, Artificial Sequence
  source: /note=“Description of Artificial Sequence Synthetic polypeptide”
QVQLQQSGAELVRPGASVTLSCKASGYTFFDYEMHWVKQTPVHGLEWIGAIDPEIDGTAYNQKFKGKAILTADKSSSTAYMELRSLTSEDSAVYYCTDYYGSSYWYFDVWGTGTTVTVSS

SEQ ID NO: 56 - DNA, 359 bp, Artificial Sequence
  source: /note=“Description of Artificial Sequence Synthetic polynucleotide”
caggttcaactgcagcagtctggggctgagctggtgaggcctggggcttcagtgacgctgtcctgcaaggcttcgggctacacattttttgactatgaaatgcactgggtgaagcagacacctgtgcatggcctggaatggattggagctattgatcctgaaattgatggtactgcctacaatcagaagttcaagggcaaggccatactgactgcagacaaatcctccagcacagcctacatggagctccgcagcctgacatctgaggactctgccgtctattactgtacagattactacggtagtagctactggtacttcgatgtctggggcacagggaccacggtcaccgtctcctc

SEQ ID NO: 57 - protein, 112 aa, Artificial Sequence
  source: /note=“Description of Artificial Sequence Synthetic polypeptide”
DVMMTQTPLSLPVSLGDQASISCRSSQSLVHSNGNTYLHWFLQKPGQSPKLLIYKVSNRFSGVPDRFSGSGSGTDFTLKISRVEAEDLGVYFCSQTTHVPLTFGAGTKLELK

SEQ ID NO: 58 - DNA, 336 bp, Artificial Sequence
  source: /note=“Description of Artificial Sequence Synthetic polynucleotide”
gatgttatgatgacccaaactccactctccctgcctgtcagtcttggagatcaagcctccatctcttgcagatctagtcagagccttgtacacagtaatggaaacacctatttacattggttcctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaactacacatgttccgctcacgttcggtgctgggaccaagctggagctgaaa

SEQ ID NO: 59 - protein, 120 aa, Artificial Sequence
  source: /note=“Description of Artificial Sequence Synthetic polypeptide”
QVQLQQSGAELVRPGASVTLSCKASGYTFTDYEMHWVKQTPVHGLEWIGAIDPEIAGTAYNQKFKGKAILTADKSSSTAYMELRSLTSEDSAVYYCSRYGGNYLYYFDYWGQGTTLTVSS

SEQ ID NO: 60 - DNA, 360 bp, Artificial Sequence
  source: /note=“Description of Artificial Sequence Synthetic polynucleotide”
caggttcaactgcagcagtctggggctgagctggtgaggcctggggcttcagtgacgctgtcctgcaaggcttcgggctacacttttactgactatgaaatgcactgggtgaagcagacacctgtccatggcctggaatggattggagctattgatcctgaaattgctggtactgcctacaatcagaagttcaagggcaaggccatactgactgcagacaaatcctccagcacagcctacatggagctccgcagcctgacatctgaggactctgccgtctattactgttcaagatacggtggtaactacctttactactttgactactggggccaaggcaccactctcacagtctcctca

SEQ ID NO: 61 - protein, 112 aa, Artificial Sequence
  source: /note=“Description of Artificial Sequence Synthetic polypeptide”
DVLMTQIPLSLPVSLGDQASISCRSSQNIVYSNGNTYLEWYLQKPGQSPKLLIYKVSNRFSGVPDRFSGSGSGTDFTLKISRVEAEDLGVYYCFQGSHVPFTFGSGTKLEIK

SEQ ID NO: 62 - DNA, 336 bp, Artificial Sequence
  source: /note=“Description of Artificial Sequence Synthetic polynucleotide”
gatgttttgatgacccaaattccactctccctgcctgtcagtcttggagatcaagcctccatctcttgcagatctagtcagaacattgtgtatagtaatggaaacacctatttagagtggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccattcacgttcggctcggggacaaagttggaaataaaa

SEQ ID NO: 63 - protein, 118 aa, Artificial Sequence
  source: /note=“Description of Artificial Sequence Synthetic polypeptide”
QVQLQQSGAELVRPGASVTLSCKASGYTFTDYEMHWVKQTPVHGLEWIGAIDPEIGGSAYNQKFKGRAILTADKSSSTAYMELRSLTSEDSAVYYCTGYDGYFWFAYWGQGTLVTVSS

SEQ ID NO: 64 - DNA, 354 bp, Artificial Sequence
  source: /note=“Description of Artificial Sequence Synthetic polynucleotide”
caggttcaactgcagcagtccggggctgagctggtgaggcctggggcttcagtgacgctgtcctgcaaggcttcgggctacacatttactgactatgaaatgcactgggtgaagcagacacctgtgcatggcctggaatggattggagctattgatcctgaaattggtggttctgcctacaatcagaagttcaagggcagggccatattgactgcagacaaatcctccagcacagcctacatggagctccgcagcctgacatctgaggactctgccgtctattattgtacgggctatgatggttacttttggtttgcttactggggccaagggactctggtcactgtctcttca

SEQ ID NO: 65 - protein, 106 aa, Artificial Sequence
  source: /note=“Description of Artificial Sequence Synthetic polypeptide”
ENVLTQSPAIMSASPGEKVTMTCSASSSVSYMHWYQQKSSTSPKLWIYDTSKLASGVPGRFSGSGSGNSYSLTISSMEAEDVATYYCFQGSGYPLTFGSGTKLEIK

SEQ ID NO: 66 - DNA, 318 bp, Artificial Sequence
  source: /note=“Description of Artificial Sequence Synthetic polynucleotide”
gaaaatgttctcacccagtctccagcaatcatgtccgcatctccaggggaaaaggtcaccatgacctgcagtgctagctcaagtgtaagttacatgcactggtaccagcagaagtcaagcacctcccccaaactctggatttatgacacatccaaactggcttctggagtcccaggtcgcttcagtggcagtgggtctggaaactcttactctctcacgatcagcagcatggaggctgaagatgttgccacttattactgttttcaggggagtgggtacccactcacgttcggctcggggacaaagttggaaataaaa

SEQ ID NO: 67 - protein, 116 aa, Artificial Sequence
  source: /note=“Description of Artificial Sequence Synthetic polypeptide”
QVQLQQSGAELVRPGASVTLSCKASGYTFTDYEMHWVKQTPVHGLEWIGGIDPETGGTAYNQKFKGKAILTADKSSSTAYMELRSLTSEDSAVYYCTSYYGSRVFWGTGTTVTVSS

SEQ ID NO: 68 - DNA, 348 bp, Artificial Sequence
  source: /note=“Description of Artificial Sequence Synthetic polynucleotide”
caggttcaactgcagcagtctggggctgagctggtgaggcctggggcttcagtgacgctgtcctgcaaggcttcgggctacacatttactgactatgaaatgcactgggtgaaacagacacctgtgcatggcctggaatggattggaggtattgatcctgaaactggtggtactgcctacaatcagaagttcaagggtaaggccatactgactgcagacaaatcctccagcacagcctacatggagctccgcagcctgacatctgaggactctgccgtctattactgtacaagttactatggtagtagagtcttctggggcacagggaccacggtcaccgtctcctca

SEQ ID NO: 69 - protein, 108 aa, Artificial Sequence
  source: /note=“Description of Artificial Sequence Synthetic polypeptide”
QIVLSQSPAILSAFPGEKVTMTCRASSSVSYMHWYQQKPGSSPKPWIYATSNLASGVPARFSGSGSGTSYSLTISSVEAEDAATYYCQQWSSNPPTLTFGAGTKLELK

SEQ ID NO: 70 - DNA, 324 bp, Artificial Sequence
  source: /note=“Description of Artificial Sequence Synthetic polynucleotide”
caaattgttctctcccagtctccagcaatcctgtctgcatttccaggggagaaggtcactatgacttgcagggccagctcaagtgtaagttacatgcactggtaccagcagaagccaggatcctcccccaaaccctggatttatgccacatccaacctggcttctggagtccctgctcgcttcagtggcagtgggtctgggacctcttactctctcacaatcagcagtgtggaggctgaagatgctgccacttattactgccagcagtggagtagtaacccacccacgctcacgttcggtgctgggaccaagctggagctgaaa

SEQ ID NO: 71 - protein, 124 aa, Artificial Sequence
  source: /note=“Description of Artificial Sequence Synthetic polypeptide”
QVQLQQSGAELARPGASVKLSCKASGYTFTSYGISWVKQRTGQGLEWIGEIYPRSGNTYYNESFKGKVTLTADKSSGTAYMELRSLTSEDSAVYFCARWGSYGSPPFYYGMDYWGQGTSVTVSS

SEQ ID NO: 72 - DNA, 372 bp, Artificial Sequence
  source: /note=“Description of Artificial Sequence Synthetic polynucleotide”
caggttcagctgcagcagtctggagctgagctggcgaggcctggggcttcagtgaagctgtcctgcaaggcttctggctacaccttcacaagctatggtataagctgggtgaagcagaggactggacagggccttgagtggattggagagatttatcctagaagtggtaatacttactacaatgagagcttcaagggcaaggtcacactgaccgcagacaaatcttccggcacagcgtacatggagctccgcagcctgacatctgaggactctgcggtctatttctgtgcaagatggggctcctacggtagtccccccttttactatggtatggactactggggtcaaggaacctcagtcaccgtctcctca

SEQ ID NO: 73 - protein, 112 aa, Artificial Sequence
  source: /note=“Description of Artificial Sequence Synthetic polypeptide”
DVLMTQTPLSLPVSLGNQASISCRSSQSIVHSSGSTYLEWYLQKPGQSPKLLIYKVSNRFSGVPDRFSGSGSGTDFTLKISRVEAEDLGVYYCFQGSHVPYTFGGGTKLEIK

SEQ ID NO: 74 - DNA, 336 bp, Artificial Sequence
  source: /note=“Description of Artificial Sequence Synthetic polynucleotide”
gatgttttgatgacccaaactccactctccctgcctgtcagtcttggaaatcaagcctccatctcttgcagatctagtcagagcattgtacatagtagtggaagcacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggctcacatgttccatacacgttcggaggggggaccaagctggaaataaaa

SEQ ID NO: 75 - protein, 123 aa, Artificial Sequence
  source: /note=“Description of Artificial Sequence Synthetic polypeptide”
QVQLQQSGAELARPGTSVKVSCKASGYTFTSYGISWVKQRIGQGLEWIGEIHPRSGNSYYNEKIRGKATLTADKSSSTAYMELRSLISEDSAVYFCARLITTVVANYYAMDYWGQGTSVTVSS

SEQ ID NO: 76 - DNA, 369 bp, Artificial Sequence
  source: /note=“Description of Artificial Sequence Synthetic polynucleotide”
caggttcagctgcagcagtctggagctgagctggcgaggcctgggacttcagtgaaggtgtcctgcaaggcttctggctataccttcacaagttatggtataagctgggtgaagcagagaattggacagggccttgagtggattggagagattcatcctagaagtggtaatagttactataatgagaagatcaggggcaaggccacactgactgcagacaaatcctccagcacagcgtacatggagctccgcagcctgatatctgaggactctgcggtctatttctgtgcaaggctgattactacggtagttgctaattactatgctatggactactggggtcaaggaacctcagtcaccgtctcctca

SEQ ID NO: 77 - protein, 112 aa, Artificial Sequence
  source: /note=“Description of Artificial Sequence Synthetic polypeptide”
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLVTFGAGTKLELK

SEQ ID NO: 78 - DNA, 336 bp, Artificial Sequence
  source: /note=“Description of Artificial Sequence Synthetic polynucleotide”
gacattgtgatgtcacagtctccatcctccctggctgtgtcagcaggagagaaggtcactatgagctgcaaatccagtcagagtctgctcaacagtagaacccgaaagaactacttggcttggtaccagcagaaaccagggcagtctcctaaactgctgatctactgggcatccactagggaatctggggtccctgatcgcttcacaggcagtggatctgggacagatttcactctcaccatcagcagtgtgcaggctgaagacctggcagtttattactgcaaacaatcttataatctggtcacgttcggtgctgggaccaagctggagctgaaa

SEQ ID NO: 79 - protein, 120 aa, Artificial Sequence; identical to SEQ ID NO: 55
  source: /note=“Description of Artificial Sequence Synthetic polypeptide”

SEQ ID NO: 80 - DNA, 359 bp, Artificial Sequence; identical to SEQ ID NO: 56
  source: /note=“Description of Artificial Sequence Synthetic polynucleotide”

SEQ ID NO: 81 - protein, 106 aa, Artificial Sequence
  source: /note=“Description of Artificial Sequence Synthetic polypeptide”
QIVLTQSPAIMSASPGEKVTISCSASSSVSYMYWYQQKPGSSPKPWIYRTSNLASGVPARFSGSGSGTSYSLTISSMEAEDAATYYCQQYHSYPLTFGAGTKLELK

SEQ ID NO: 82 - DNA, 318 bp, Artificial Sequence
  source: /note=“Description of Artificial Sequence Synthetic polynucleotide”
caaattgttctcacccagtctccagcaatcatgtctgcatctccaggggagaaggtcaccatatcctgcagtgccagctcaagtgtaagttacatgtactggtaccagcagaagccaggatcctcccccaaaccctggatttatcgcacatccaacctggcttctggagtccctgctcgcttcagtggcagtgggtctgggacctcttactctctcacaatcagcagcatggaggctgaagatgctgccacttattactgccagcagtatcatagttacccactcacgttcggtgctgggaccaagctggagctgaaa

SEQ ID NO: 83 - protein, 109 aa, Artificial Sequence
  source: /note=“Description of Artificial Sequence Synthetic polypeptide”
QIVLTQSPAIMSASPGERVTMTCSASSSVSSSYLYWYQQKSGSSPKLWIYSISNLASGVPARFSGSGSGTSYSLTINSMEAEDAATYYCQQWSSNPQLTFGAGTKLELK

SEQ ID NO: 84 - DNA, 327 bp, Artificial Sequence
  source: /note=“Description of Artificial Sequence Synthetic polynucleotide”
caaattgttctcacccagtctccagcaatcatgtctgcatctcctggggaacgggtcaccatgacctgcagtgccagctcaagtgtaagttccagctacttgtactggtaccagcagaagtcaggatcctccccaaaactctggatttatagcatatccaacctggcttctggagtcccagctcgcttcagtggcagtgggtctgggacctcttactctctcacaatcaacagcatggaggctgaagatgctgccacttattactgccagcagtggagtagtaacccacagctcacgttcggtgctgggaccaagctggagctgaaa

SEQ ID NO: 85 - protein, 121 aa, Artificial Sequence
  source: /note=“Description of Artificial Sequence Synthetic polypeptide”
QVQLKQSGAELVKPGASVKISCKASGYTFTDYYINWVKQRPGQGLEWIGKIGPGSGSTYYNEKFKGKATLTADKSSSTAYMQLSSLTSEDSAVYFCARTGYYVGYYAMDYWGQGTSVTVSS

SEQ ID NO: 86 - DNA, 363 bp, Artificial Sequence
  source: /note=“Description of Artificial Sequence Synthetic polynucleotide”
caggtccagctgaagcagtctggagctgagctggtgaagcctggggcttcagtgaagatatcctgcaaggcttctggctacaccttcactgactactatataaactgggtgaagcagaggcctggacagggccttgagtggattggaaagattggtcctggaagtggtagtacttactacaatgagaagttcaagggcaaggccacactgactgcagacaaatcctccagcacagcctacatgcagctcagcagcctgacatctgaggactctgcagtctatttctgtgcaagaactggttactacgttggttactatgctatggactactggggtcaaggaacctcagtcaccgtctcctca

SEQ ID NO: 87 - protein, 118 aa, Artificial Sequence
  source: /note=“Description of Artificial Sequence Synthetic polypeptide”
QVQLQQSGAELARPGASVKLSCKASGYTFTIYGISWVKQRTGQGLEWIGEIYPRSDNTYYNEKFKGKATLTADKSSSTAYMELRSLTSEDSAVYFCARWYSFYAMDYWGQGTSVTVSS

SEQ ID NO: 88 - DNA, 354 bp, Artificial Sequence
  source: /note=“Description of Artificial Sequence Synthetic polynucleotide”
caggttcagctgcagcagtctggagctgagctggcgaggcctggggcttcagtgaagctgtcctgcaaggcttctggctacaccttcacaatctatggtataagctgggtgaaacagagaactggacagggccttgagtggattggagagatttatcctagaagtgataatacttactacaatgagaagttcaagggcaaggccacactgactgcagacaaatcctccagcacagcgtacatggagctccgcagcctgacatctgaggactctgcggtctatttctgtgcaagatggtactcgttctatgctatggactactggggtcaaggaacctcagtcaccgtctcctca

SEQ ID NO: 89 - protein, 111 aa, Artificial Sequence
  source: /note=“Description of Artificial Sequence Synthetic polypeptide”
EVQLVESGGGLVQPGGSLRLSCAASGGDWSANFMYWYRQAPGKQRELVARISGRGVVDYVESVKGRFTISRDNSKNTLYLQMNSLRAEDTAVYYCAVASYWGQGTLVTVSS

SEQ ID NO: 90 - protein, 119 aa, Artificial Sequence
  source: /note=“Description of Artificial Sequence Synthetic polypeptide”
EVQLVESGGGLVQPGGSLRLSCAASGSTSSINTMYWYRQAPGKERELVAFISSGGSTNVRDSVKGRFTISRDNSKNTLYLQMNSLRAEDTAVYYCNTYIPYGGTLHDFWGQGTLVTVSS

SEQ ID NO: 91 - protein, 116 aa, Artificial Sequence
  source: /note=“Description of Artificial Sequence Synthetic polypeptide”
QVQLVESGGGVVQAGGSLRLSCAASGSTFSIRAMRWYRQAPGTERDLVAVIYGSSTYYADAVKGRFTISRDNSKNTLYLQMNSLRAEDTAVYYCNADTIGTARDYWGQGTLVTVSS

SEQ ID NO: 92 - protein, 58 aa, Homo sapiens
NFSPLARRVDRVAIYEEFLRMTRNGTQLQNFTLDRSSVLVDGYSPNRNEPLTGNSDLP

SEQ ID NO: 93 - protein, 59 aa, Artificial Sequence
  source: /note=“Description of Artificial Sequence Synthetic polypeptide”
NFSPLARRVDRVAIYEEFLRMTRNGTQLQNFTLDRSSVLVDGYSPNRNEPLTGNSDLPC

SEQ ID NO: 94 - DNA, 29 bp, Artificial Sequence
  source: /note=“Description of Artificial Sequence Synthetic primer”
gatgtgcagctgcaggagtctggrggagg

SEQ ID NO: 95 - DNA, 34 bp, Artificial Sequence
  source: /note=“Description of Artificial Sequence Synthetic primer”
ctagtgcggccgctgaggagacggtgacctgggt

SEQ ID NO: 96 - DNA, 22 bp, Artificial Sequence
  source: /note=“Description of Artificial Sequence Synthetic primer”
tcacacaggaaacagctatgac

SEQ ID NO: 97 - DNA, 24 bp, Artificial Sequence
  source: /note=“Description of Artificial Sequence Synthetic primer”
cgccagggttttcccagtcacgac

SEQ ID NO: 98 - RNA, 6 nt, Artificial Sequence
  source: /note=“Description of Artificial Sequence Synthetic oligonucleotide”
aauaaa

SEQ ID NO: 99 - DNA, 20 bp, Artificial Sequence
  source: /note=“Description of Artificial Sequence Synthetic primer”
ttatgcttccggctcgtatg

SEQ ID NO: 100 - protein, 20 aa, Artificial Sequence
  source: /note=“Description of Artificial Sequence Synthetic peptide”
  SITE (1)..(20): /note=“This sequence may encompass 1-4 ′Gly Gly Gly Gly Ser′ repeating units”
GGGGSGGGGSGGGGSGGGGS

SEQ ID NO: 101 - protein, 20 aa, Artificial Sequence
  source: /note=“Description of Artificial Sequence Synthetic peptide”
  SITE (1)..(20): /note=“This sequence may encompass 2-4 ′Gly Gly Gly Gly Ser′ repeating units”
GGGGSGGGGSGGGGSGGGGS

SEQ ID NO: 102 - protein, 15 aa, Artificial Sequence
  source: /note=“Description of Artificial Sequence Synthetic peptide”
  SITE (1)..(15): /note=“This sequence may encompass 1-3 ′Gly Gly Gly Gly Ser′ repeating units”
GGGGSGGGGSGGGGS

SEQ ID NO: 103 - RNA, 2000 nt, Artificial Sequence
  Description of Artificial Sequence Synthetic polynucleotide
  misc_feature (1)..(2000): /note=“This sequence may encompass 50-2000 nucleotides”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 104 - protein, 20 aa, Artificial Sequence
  source: /note=“Description of Artificial Sequence Synthetic peptide”
GGGGSGGGGSGGGGSGGGGS

SEQ ID NO: 105 - protein, 15 aa, Artificial Sequence
  source: /note=“Description of Artificial Sequence Synthetic peptide”
GGGGSGGGGSGGGGS

SEQ ID NO: 106 - protein, 4 aa, Artificial Sequence
  source: /note=“Description of Artificial Sequence Synthetic peptide”
  source: /note=“See specification as filed for detailed description of substitutions and preferred embodiments”
GGGS

SEQ ID NO: 107 - RNA, 5000 nt, Artificial Sequence
  source: /note=“Description of Artificial Sequence Synthetic polynucleotide”
  misc_feature (1)..(5000): /note=“This sequence may encompass 50-5000 nucleotides”
  source: /note=“See specification as filed for detailed description of substitutions and preferred embodiments”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 108 - protein, 30 aa, Artificial Sequence
  source: /note=“Description of Artificial Sequence Synthetic polypeptide”
  SITE (1)..(30): /note=“This sequence may encompass 1-6 ′Gly Gly Gly Gly Ser′ repeating units”
  source: /note=“See specification as filed for detailed description of substitutions and preferred embodiments”
GGGGSGGGGSGGGGSGGGGSGGGGSGGGGS

SEQ ID NO: 109 - protein, 5 aa, Artificial Sequence
  source: /note=“Description of Artificial Sequence Synthetic peptide”
  source: /note=“See specification as filed for detailed description of substitutions and preferred embodiments”
GGGGS

SEQ ID NO: 110 - protein, 59 aa, Artificial Sequence; identical to SEQ ID NO: 93
  source: /note=“Description of Artificial Sequence Synthetic polypeptide”

SEQ ID NO: 111 - DNA, 30 bp, Artificial Sequence
  source: /note=“Description of Artificial Sequence Synthetic oligonucleotide”
ggtggcggaggttctggaggtggaggttcc

SEQ ID NO: 112 - DNA, 100 bp, Artificial Sequence
  source: /note=“Description of Artificial Sequence Synthetic polynucleotide”
tttttttttttttttttttttttttttttttttttttttttttttttttttttttttttttttttttttttttttttttttttttttttttttttttttt

SEQ ID NO: 113 - DNA, 5000 bp, Artificial Sequence
  source: /note=“Description of Artificial Sequence Synthetic polynucleotide”
  misc_feature (1)..(5000): /note=“This sequence may encompass 50-5000 nucleotides”
tttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttt

SEQ ID NO: 114 - RNA, 5000 nt, Artificial Sequence; identical to SEQ ID NO: 107
  source: /note=“Description of Artificial Sequence Synthetic polynucleotide”
  misc_feature (1)..(5000): /note=“This sequence may encompass 100-5000 nucleotides”

SEQ ID NO: 115 - RNA, 400 nt, Artificial Sequence
  source: /note=“Description of Artificial Sequence Synthetic polynucleotide”
  misc_feature (1)..(400): /note=“This sequence may encompass 100-400 nucleotides”
  source: /note=“See specification as filed for detailed description of substitutions and preferred embodiments”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 116 - protein, 10 aa, Artificial Sequence
  source: /note=“Description of Artificial Sequence Synthetic peptide”
GGGGSGGGGS

SEQ ID NO: 117 - protein, 6 aa, Artificial Sequence
  source: /note=“Description of Artificial Sequence Synthetic 6xHis tag”
HHHHHH